(19) United States
(12) Patent Application Publication
(10) Pub. No.: US 2025/0312150 A1
(43) Pub. Date: Oct. 9, 2025

(54) SYSTEMS, APPARATUSES, AND METHODS FOR PROSTHETIC VALVES

(71) Applicant: EDWARDS LIFESCIENCES CORPORATION, Irvine, CA (US)

(72) Inventors: David Robert Landon, Huntington Beach, CA (US); Kevin M. Golemo, Mission Viejo, CA (US); Eric Robert Dixon, Villa Park, CA (US); Matthew Michael Becerra, Lake Forest, CA (US); Matthew A. Peterson, Costa Mesa, CA (US); Rani Abdullah Mahmoudi, Huntington Beach, CA (US); Olivia Marie Conroy, New Rochelle, NY (US); Shane Chazhikatt Cyriac, Cypress, CA (US); Kellie Cao, Irvine, CA (US); Katherine Nickolite, Huntington Beach, CA (US); Gregory Scott Tyler, II, Winston-Salem, NC (US)

(21) Appl. No.: 19/241,653

(22) Filed: Jun. 18, 2025

Related U.S. Application Data
(63) Continuation of application No. PCT/US2023/085391, filed on Dec. 21, 2023.
(60) Provisional application No. 63/436,051, filed on Dec. 29, 2022.
(60) Provisional application No. 63/533,458, filed on Aug. 18, 2023.

(51) Int. Cl. A61F 2/24; A61F 2/00; A61F 2/848; A61F 2/90; A61N 1/362; A61N 1/375

(52) U.S. Cl. CPC A61F 2/2418; A61N 1/362; A61N 1/37516; A61F 2002/0081; A61F 2002/8483; A61F 2/90; A61F 2210/0076; A61F 2230/0078

(57) ABSTRACT

A prosthetic heart valve includes a self-expanding frame and a plurality of prosthetic valve leaflets positioned within an interior of the frame. Ventricular anchors extend from a downstream portion of the frame and are shaped for capturing native leaflets. Barbs are preferably provided along an outer surface of the frame for enhanced fixation. The self-expanding frame may include an inner frame for supporting the prosthetic valve leaflets and an outer frame for sealing against the tissue of the native heart valve. The inner and outer frames may be attached via a flexible intermediate component for allowing the inner frame to move relative to the outer frame. The outer frame may be more conformable than the inner frame and the outer frame may include axial recesses for receiving the ventricular anchors and thereby reducing profile after implantation. The prosthetic heart valve is preferably sized to replace a native tricuspid valve.

### CROSS REFERENCE TO RELATED APPLICATIONS

This application is a continuation of International Application No. PCT/US2023/085391, filed Dec. 21, 2023, which designates the United States and was published in English by the International Bureau on Jul. 4, 2024, which claims the benefit of U.S. Provisional Application No. 63/436,051, filed Dec. 29, 2022, and U.S. Provisional Application No. 63/533,458, filed Aug. 18, 2023, the entire contents of each of which are hereby incorporated by reference.

### BACKGROUND

### Field

Certain examples disclosed herein relate generally to prostheses for implantation within a lumen or body cavity. In particular, the prostheses relate in some examples to replacement heart valves, such as replacement mitral heart valves or replacement tricuspid heart valves.

### Description of the Related Art

Human heart valves, which include the aortic, pulmonary, mitral and tricuspid valves, function essentially as one-way valves operating in synchronization with the pumping heart. The valves allow blood to flow downstream, but block blood from flowing upstream. Diseased heart valves exhibit impairments such as narrowing of the valve or regurgitation, which inhibit the valves' ability to control blood flow. Such impairments reduce the heart's blood-pumping efficiency and can be a debilitating and life-threatening condition. For example, valve insufficiency can lead to conditions such as heart hypertrophy and dilation of the ventricle. Thus, extensive efforts have been made to develop methods and apparatuses to repair or replace impaired heart valves.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open heart surgery. Replacement valves are being designed to be delivered through minimally invasive procedures and even percutaneous procedures. Such replacement valves often include prosthetic valve leaflets that are connected to an expandable frame that is then delivered to the native valve's annulus.

Development of prostheses including but not limited to replacement heart valves that can be compacted for delivery and then controllably expanded for controlled placement has proven to be particularly challenging. An additional challenge relates to the ability of such prostheses to be secured relative to intralumenal tissue, e.g., tissue within any body lumen or cavity, in an atraumatic manner.

Delivering a prosthesis to a desired location in the human body, for example delivering a replacement heart valve to the mitral valve, can also be challenging. Obtaining access to perform procedures in the heart or in other anatomical locations may require delivery of devices percutaneously through tortuous vasculature or through open or semi-open surgical procedures. The ability to control the deployment of the prosthesis at the desired location can also be challenging.

### SUMMARY

Examples of the present disclosure may be directed to an implant, which may comprise a prosthesis such as but not limited to a replacement heart valve. The replacement heart valve may comprise a replacement mitral heart valve or replacement tricuspid heart valve. In some examples, a replacement heart valve and methods for delivering a replacement heart valve to a native heart valve, such as a mitral valve, an aortic valve, or a tricuspid valve, are provided.

Improved anchoring of a prosthetic valve to a native implantation site may be disclosed herein. Improved interaction between tissue of a native implantation site and a prosthetic valve may be disclosed herein. Configurations of prosthetic heart valves to accommodate a pacemaker lead may be disclosed herein.

Examples of the present disclosure may include a prosthetic heart valve for replacing the function of a native heart valve. The prosthetic heart valve may include a self-expanding frame sized for deployment within the native heart valve, the frame having an outer surface for pressing against tissue of the native heart valve. The prosthetic heart valve may include a plurality of prosthetic valve leaflets positioned within an interior of the frame, the leaflets configured to allow flow in a first direction and to prevent flow in a second direction. The prosthetic heart valve may include a fabric skirt covering at least a portion of the outer surface of the frame. The prosthetic heart valve may include a plurality of ventricular anchors extending from a downstream portion of the frame and shaped for capturing native leaflets of the native heart valve between the anchors and the outer surface of the frame. A plurality of barbs may be provided along the outer surface of the frame and wherein the ventricular anchors press the native leaflets against the barbs for securing the prosthetic heart valve within the native heart valve.

Examples of the present disclosure may include a prosthetic valve configured to be deployed to a native valve. The prosthetic valve may include one or more prosthetic valve leaflets, and a valve body supporting the one or more prosthetic valve leaflets, wherein at least a portion of the prosthetic valve includes a material configured to reduce tissue formation or thrombus formation along the portion of the prosthetic valve.

Examples of the present disclosure may include a method. The method may include deploying a prosthetic valve to a native valve. The prosthetic valve may include one or more prosthetic valve leaflets, and a valve body supporting the one or more prosthetic valve leaflets, wherein at least a portion of the prosthetic valve includes a material configured to reduce tissue formation or thrombus formation along the portion of the prosthetic valve.

Examples of the present disclosure may include a prosthetic valve configured to be deployed to a native valve. The prosthetic valve may comprise one or more prosthetic valve leaflets, and a valve body supporting the one or more prosthetic valve leaflets, wherein at least a portion of the prosthetic valve includes a frame having a rough surface.

Examples of the present disclosure may include a method. The method may comprise deploying a prosthetic valve to a native valve. The prosthetic valve may include one or more prosthetic valve leaflets, and a valve body supporting the one or more prosthetic valve leaflets, wherein at least a portion of the prosthetic valve includes a frame having a rough surface.

Examples of the present disclosure may include a prosthetic valve configured to be deployed to a native valve having native valve leaflets. The prosthetic valve may include one or more prosthetic valve leaflets, a valve body supporting the one or more prosthetic valve leaflets, the valve body including one or more grip features configured to be positioned radially inward of one or more of the native valve leaflets, and one or more anchors configured to be positioned radially outward of the one or more of the native valve leaflets and press the one or more of the native valve leaflets towards the one or more grip features to reduce movement of the one or more of the native valve leaflets relative to the valve body.

Examples of the present disclosure may include a method. The method may comprise deploying a prosthetic valve to a native valve. The prosthetic valve may include one or more prosthetic valve leaflets, a valve body supporting the one or more prosthetic valve leaflets, the valve body including one or more grip features configured to be positioned radially inward of one or more of the native valve leaflets, and one or more anchors configured to be positioned radially outward of the one or more of the native valve leaflets and press the one or more of the native valve leaflets towards the one or more grip features to reduce movement of the one or more of the native valve leaflets relative to the valve body.

Examples of the present disclosure may include a prosthetic valve configured to be deployed to a native valve having native valve leaflets. The prosthetic valve may include one or more prosthetic valve leaflets, a valve body supporting the one or more prosthetic valve leaflets, and one or more anchors extending from the valve body and configured to be positioned radially outward of one or more of the native valve leaflets to capture the one or more of the native valve leaflets. At least a portion of the prosthetic valve may include one or more grip features configured to engage a surface of the native valve upon one of the anchors failing to capture the one or more native valve leaflets.

Examples of the present disclosure may include a method. The method may comprise deploying a prosthetic valve to a native valve. The prosthetic valve may include one or more prosthetic valve leaflets, a valve body supporting the one or more prosthetic valve leaflets, and one or more anchors extending from the valve body and configured to be positioned radially outward of one or more of the native valve leaflets to capture the one or more of the native valve leaflets. At least a portion of the prosthetic valve may include one or more grip features configured to engage a surface of the native valve upon one of the anchors failing to capture the one or more native valve leaflets.

Examples of the present disclosure may include a prosthetic valve configured to be deployed to a native valve. The prosthetic valve may include one or more prosthetic valve leaflets and a valve body supporting the one or more prosthetic valve leaflets and having an outer surface, wherein the outer surface of the valve body includes a channel for a pacemaker lead to be passed through.

Examples of the present disclosure may include a method. The method may comprise deploying a prosthetic valve to a native valve. The prosthetic valve may include one or more prosthetic valve leaflets, and a valve body supporting the one or more prosthetic valve leaflets and having an outer surface, wherein the outer surface of the valve body includes a channel for a pacemaker lead to be passed through.

Examples of the present disclosure may include a prosthetic valve configured to be deployed to a native valve. The prosthetic valve may include one or more prosthetic valve leaflets and a valve body. The valve body may include an inner frame supporting the one or more prosthetic valve leaflets and having a proximal end portion and a distal end portion, and an outer frame positioned radially outward of the inner frame and having a proximal end portion and a distal end portion and an outer surface facing radially outward from the prosthetic valve, the proximal end portion of the outer frame being coupled to the proximal end portion of the inner frame, and the distal end portion of the outer frame being spaced from the inner frame with a gap. The prosthetic valve may include a plurality of anchors each coupled to the inner frame and having a hook shape and extending radially outward from the inner frame, a first one of the plurality of anchors having a tip positioned radially outward of the outer frame and overlapping the outer surface, a second one of the plurality of anchors having a tip positioned distal of the outer frame and at least partially recessed radially inward of the outer surface.

Examples of the present disclosure may include a method. The method may comprise deploying a prosthetic valve to a native valve. The prosthetic valve may include one or more prosthetic valve leaflets, and a valve body including: an inner frame supporting the one or more prosthetic valve leaflets and having a proximal end portion and a distal end portion, and an outer frame positioned radially outward of the inner frame and having a proximal end portion and a distal end portion and an outer surface facing radially outward from the prosthetic valve, the proximal end portion of the outer frame being coupled to the proximal end portion of the inner frame, and the distal end portion of the outer frame being spaced from the inner frame with a gap, and a plurality of anchors each coupled to the inner frame and having a hook shape and extending radially outward from the inner frame, a first one of the plurality of anchors having a tip positioned radially outward of the outer frame and overlapping the outer surface, a second one of the plurality of anchors having a tip positioned distal of the outer frame and at least partially recessed radially inward of the outer surface.

Examples of the present disclosure may include a prosthetic valve configured to be deployed to a native valve. The prosthetic valve may include one or more prosthetic valve leaflets and a valve body. The prosthetic valve may include one or more hook arm anchors coupled to the valve body and each adapted to hook over a leaflet of the native valve to anchor to the native valve; and one or more clasp anchors coupled to the valve body and each adapted to clasp a portion of the native valve to anchor to the native valve.

Examples of the present disclosure may include a method. The method may comprise deploying a prosthetic valve to a native valve. The prosthetic valve may include one or more prosthetic valve leaflets, a valve body supporting the one or more prosthetic valve leaflets, one or more hook arm anchors coupled to the valve body and each adapted to hook over a leaflet of the native valve to anchor to the native valve, and one or more clasp anchors coupled to the valve body and each adapted to clasp a portion of the native valve to anchor to the native valve.

Examples of the present disclosure may include a prosthetic valve configured to be deployed to a native valve. The prosthetic valve may include one or more prosthetic valve leaflets and a valve body supporting the one or more prosthetic valve leaflets and having a proximal end portion and a distal end portion. The prosthetic valve may include one or more hook arm anchors coupled to the valve body and each adapted to hook over a leaflet of the native valve to anchor to the native valve; and one or more support arms coupled to the valve body and each having a proximal end portion coupled to the valve body and a distal end portion protruding in a distal direction from the valve body and configured to extend into the ventricle, the one or more support arms adapted to stabilize the prosthetic valve within the native valve.

Examples of the present disclosure may include a method. The method may comprise deploying a prosthetic valve to a native valve. The prosthetic valve may include one or more prosthetic valve leaflets, a valve body supporting the one or more prosthetic valve leaflets and having a proximal end portion and a distal end portion, one or more hook arm anchors coupled to the valve body and each adapted to hook over a leaflet of the native valve to anchor to the native valve, and one or more support arms coupled to the valve body and each having a proximal end portion coupled to the valve body and a distal end portion protruding in a distal direction from the valve body and configured to extend into the ventricle, the one or more support arms adapted to stabilize the prosthetic valve within the native valve.

### BRIEF DESCRIPTION OF THE DRAWINGS

FIG. 1 shows a perspective view of a prosthetic valve.

FIG. 2 shows a cross sectional schematic view of the prosthetic valve shown in FIG. 1.

FIG. 3 shows a perspective view of a barrier layer for prosthetic valve leaflets.

FIG. 4 shows an upper perspective view of a barrier layer coupled to an inner frame of a prosthetic valve.

FIG. 5A illustrates a cross sectional schematic view of a prosthetic valve deployed to an implantation site.

FIG. 5B illustrates a cross sectional schematic view of a prosthetic valve deployed to an implantation site.

FIG. 6 illustrates a cross sectional schematic view of a prosthetic valve deployed to an implantation site.

FIG. 7A illustrates a perspective view of a skirt.

FIG. 7B illustrates a side cross sectional view of the skirt shown in FIG. 7A.

FIG. 7C illustrates a proximal perspective view of the skirt shown in FIG. 7A coupled to an outer skirt of a prosthetic valve.

FIG. 7D illustrates a distal perspective view of the skirt shown in FIG. 7A coupled to an outer skirt of a prosthetic valve.

FIG. 7E illustrates a distal perspective view of the skirt shown in FIG. 7A coupled to a frame.

FIG. 7F illustrates a side cross sectional view of a wall of the skirt shown in FIG. 7A.

FIG. 7G illustrates a side cross sectional view of a prosthetic valve including the skirt shown in FIG. 7A.

FIG. 8 illustrates a perspective view of a frame for a prosthetic valve.

FIG. 9 illustrates a perspective view of the outer frame shown in FIG. 8 separate from the inner frame shown in FIG. 8.

FIG. 10 illustrates a perspective view of the inner frame shown in FIG. 8 separate from the outer frame shown in FIG. 8.

FIG. 11 illustrates a perspective view of a prosthetic valve.

FIG. 12 illustrates a plan view of a portion of a frame of a prosthetic valve.

FIG. 13 illustrates a detail view of a portion of the frame circled in FIG. 12.

FIG. 14 illustrates a side cross sectional schematic view of the portion of the frame shown in FIG. 13.

FIG. 15 illustrates a perspective view of a portion of a frame.

FIG. 16 illustrates a side cross sectional schematic view of the portion of the frame shown in FIG. 15.

FIG. 17 illustrates a perspective view of a portion of a frame.

FIG. 18 illustrates a side cross sectional schematic view of the portion of the frame shown in FIG. 17.

FIG. 19 illustrates a plan view of a portion of a frame of a prosthetic valve.

FIG. 20 illustrates a side cross sectional view of a prosthetic valve deployed to an implantation site.

FIG. 21A illustrates a plan view of a position of an anchor relative to a grip feature.

FIG. 21B illustrates a side cross sectional schematic view of the position of the anchor shown in FIG. 21A.

FIG. 22A illustrates a cross sectional schematic view of a prosthetic valve being deployed to a native valve.

FIG. 22B illustrates a cross sectional schematic view of the prosthetic valve shown in FIG. 22A deployed to a native valve.

FIG. 23 illustrates a plan view of a portion of a frame of a prosthetic valve.

FIG. 24A illustrates a plan view of a position of an anchor relative to a grip feature.

FIG. 24B illustrates a side cross sectional schematic view of a position of the anchor shown in FIG. 24A.

FIG. 25 illustrates a plan view of a portion of a frame of a prosthetic valve.

FIG. 26 illustrates a perspective view of a portion of a frame of a prosthetic valve.

FIG. 27 illustrates a perspective view of an anchor positioned relative to a grip feature.

FIG. 28 illustrates a side view of an anchor positioned relative to a grip feature.

FIG. 29A illustrates a side view of a frame including a plurality of grip features.

FIG. 29B illustrates a side cross sectional view of an anchor positioned relative to one of the grip features shown in FIG. 29A.

FIG. 30A illustrates a perspective view of a prosthetic valve.

FIG. 30B illustrates a side cross sectional view of an anchor positioned relative to one of the grip features of the prosthetic valve shown in FIG. 30A.

FIG. 31 illustrates a side cross sectional view of the prosthetic valve shown in FIG. 30A deployed to a native valve.

FIG. 32 illustrates a distal perspective view of a prosthetic valve.

FIG. 33 illustrates a cross sectional schematic view of a prosthetic valve deployed to a native valve.

FIG. 34 illustrates a cross sectional schematic view of a prosthetic valve in an undeployed configuration and positioned within a capsule.

FIG. 35 illustrates a perspective view of a prosthetic valve being deployed from a capsule.

FIG. 36 illustrates a side view of a grip feature on an anchor.

FIG. 37A illustrates a side view of a grip feature on an anchor.

FIG. 37B illustrates a perspective view of a grip feature for an anchor.

FIG. 37C illustrates a perspective view of a grip feature for an anchor.

FIG. 38 illustrates a side view of a grip feature for an anchor.

FIG. 39A illustrates a perspective view of a grip feature for an anchor.

FIG. 39B illustrates a perspective view of a grip feature for an anchor.

FIG. 40A illustrates a side view of a grip feature for an anchor.

FIG. 40B illustrates a side view of a grip feature for an anchor.

FIG. 41 illustrates a side cross sectional view of a frame for a prosthetic valve.

FIG. 42 illustrates a side cross sectional view of a prosthetic valve being deployed to an implantation site.

FIG. 43 illustrates a perspective view of an anchor relative to a plurality of grip features.

FIG. 44 illustrates a perspective view of a prosthetic valve.

FIG. 45 illustrates a top view of the prosthetic valve shown in FIG. 44.

FIG. 46 illustrates a top perspective view of a pacemaker lead extending through a channel of the prosthetic valve shown in FIG. 44.

FIG. 47 illustrates a side cross sectional schematic view of the prosthetic valve shown in FIG. 44 deployed to an implantation site.

FIG. 48 illustrates a side cross sectional schematic view of a prosthetic valve deployed to an implantation site with a pacemaker lead passing along a side of the prosthetic valve.

FIG. 49 illustrates a perspective view of a prosthetic valve.

FIG. 50 illustrates a distal end view or outflow end view of the prosthetic valve of FIG. 49.

FIG. 51 illustrates a perspective view of an inner frame of the prosthetic valve of FIG. 49.

FIG. 52 illustrates a side view of an outer frame of the prosthetic valve of FIG. 49.

FIG. 53 illustrates a perspective view of the inner frame of the prosthetic valve of FIG. 49.

FIG. 54 illustrates a side schematic cross sectional view of the prosthetic valve of FIG. 49.

FIG. 55 illustrates a side schematic cross sectional view of a portion of the prosthetic valve of FIG. 49.

FIG. 56 illustrates a perspective view of a frame for a prosthetic valve.

FIG. 57 illustrates a proximal end view or inflow end view of the prosthetic valve of FIG. 56.

FIG. 58 illustrates a perspective view of a prosthetic valve.

FIG. 59 illustrates a side cross sectional view of the prosthetic valve of FIG. 58.

FIG. 60 illustrates a side view of a portion of an inner frame.

FIG. 61 illustrates a perspective view of a portion of an inner frame.

FIG. 62 illustrates a perspective view of an inner frame including the portions of FIGS. 60 and 61.

FIG. 63 illustrates a cross sectional view of a prosthetic valve utilizing the frame shown in FIG. 62.

FIG. 64A illustrates a proximal end view or inflow end view of a frame of a prosthetic valve.

FIG. 64B illustrates a perspective view of the frame shown in FIG. 64A.

FIG. 65 illustrates a side perspective view of a prosthetic valve utilizing the frame of FIG. 64A.

FIG. 66 illustrates a side cross sectional schematic view of a prosthetic valve utilizing the frame shown in FIG. 64A.

FIG. 67 illustrates a perspective view of a prosthetic valve.

FIG. 68 illustrates a proximal end view or inflow end view of the prosthetic valve shown in FIG. 67.

FIG. 69 illustrates a proximal end view or inflow end view of a frame of the prosthetic valve shown in FIG. 67.

FIG. 70 illustrates a side view of a position of an anchor tip relative to the frame shown in FIG. 69.

FIG. 71 illustrates a side cross sectional schematic view of a prosthetic valve utilizing the frame shown in FIG. 69.

FIG. 72 illustrates a side view of a frame of a prosthetic valve.

FIG. 73 illustrates a top schematic view of a prosthetic valve utilizing a frame as shown in FIG. 72.

FIG. 74 illustrates a proximal end view or inflow end view of the frame shown in FIG. 72.

FIG. 75 illustrates a side schematic cross sectional view of a prosthetic valve utilizing a frame as shown in FIG. 72.

FIG. 76 illustrates a top schematic view of a prosthetic valve.

FIG. 77 illustrates a perspective view of an inner frame including clasp anchors.

FIG. 78 illustrates a top schematic view of a prosthetic valve utilizing a frame as shown in FIG. 77.

FIG. 79 illustrates a perspective view of a prosthetic valve utilizing a frame as shown in FIG. 77.

FIG. 80 illustrates a plan view of the inner frame shown in FIG. 77.

FIG. 81 illustrates a plan view of a clasp anchor.

FIG. 82 illustrates a side view of a position of an anchor tip relative to an outer frame.

FIG. 83 illustrates a plan view of a clasp anchor.

FIG. 84 illustrates a plan view of a clasp anchor.

FIG. 85 illustrates a plan view of a clasp anchor.

FIG. 86 illustrates a side schematic cross sectional view of a prosthetic valve utilizing a frame as shown in FIG. 77.

FIG. 87 illustrates a side schematic cross sectional view of a variation of the prosthetic valve shown in FIG. 86.

FIG. 88 illustrates a schematic view of delivery of the prosthetic valve shown in FIG. 86.

FIG. 89 illustrates a schematic view of delivery of the prosthetic valve shown in FIG. 86.

FIG. 90 illustrates a schematic view of delivery of the prosthetic valve shown in FIG. 86.

FIG. 91 illustrates a schematic view of delivery of the prosthetic valve shown in FIG. 86.

FIG. 92 illustrates a distal or outflow perspective view of a prosthetic valve.

FIG. 93 illustrates a side perspective view of the prosthetic valve of FIG. 92.

FIG. 94 illustrates a plan view of a barbed sheet.

FIG. 95 illustrates a schematic view of delivery of the prosthetic valve shown in FIG. 92.

FIG. 96 illustrates a schematic view of delivery of the prosthetic valve shown in FIG. 92.

FIG. 97 illustrates a schematic view of delivery of a variation of the prosthetic valve shown in FIG. 92.

FIG. 98 illustrates a side view of a delivery system.

FIG. 99 illustrates a schematic view of a delivery system approaching an implantation site.

FIG. 100 illustrates a schematic view of a delivery system approaching a tricuspid valve.

FIG. 101 illustrates a schematic view of an implant being deployed to a tricuspid valve.

FIG. 102 illustrates a schematic view of an implant being deployed to a tricuspid valve.

FIG. 103 illustrates a schematic view of a tether assembly released from an implant.

FIG. 104 illustrates a side view of a prosthetic valve deployed to a tricuspid valve.

### DETAILED DESCRIPTION

The present specification and drawings provide aspects and features of the disclosure in the context of several examples of implants such as prosthetic valves or replacement heart valves, and delivery systems and methods that are configured for use in the vasculature of a patient, such as for replacement of natural heart valves in a patient. These examples may be discussed in connection with replacing specific valves such as the patient's aortic, tricuspid, or mitral valve. However, it is to be understood that the features and concepts discussed herein can be applied to products other than heart valve implants. For example, features described herein can be applied to other medical implants, for example other types of prostheses, for use elsewhere in the body, such as within an artery, a vein, or other body cavities or locations. In addition, particular features of a valve, delivery system, etc. should not be taken as limiting, and features of any one example discussed herein can be combined with features of other examples as desired and when appropriate. While certain of the examples described herein are described in connection with a transfemoral delivery approach, it should be understood that these examples can be used for other delivery approaches such as, for example, transapical or transjugular approaches. Moreover, it should be understood that certain of the features described in connection with some examples can be incorporated with other examples, including those which are described in connection with different delivery approaches.

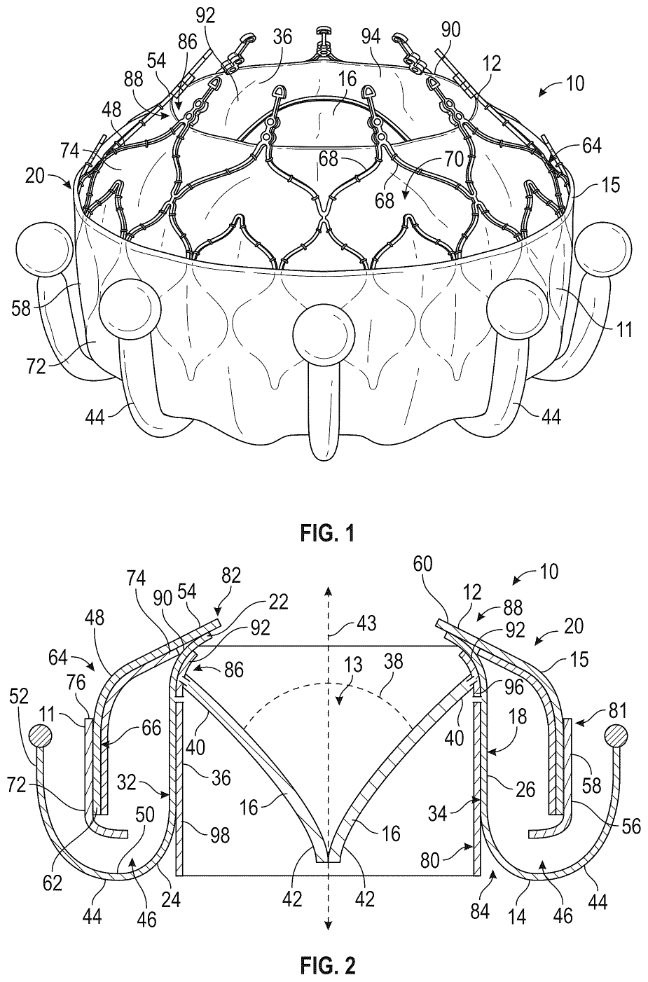

FIG. 1 illustrates a perspective view of an implant in the form of a prosthetic valve 10. The prosthetic valve 10 may comprise a prosthetic heart valve for deployment to a native heart valve of a patient's body. The prosthetic heart valve may replace the function of a native heart valve. In examples, other forms of implants and prosthetic valves may be utilized as desired.

The prosthetic valve 10 may be configured to be deployed to an annulus of a native valve, which may comprise a native mitral valve or a native tricuspid valve. In examples, other implantation locations may be utilized such as within an aortic or pulmonary valve, or in other valves or locations within a patient's body as desired.

The prosthetic valve 10 may include a proximal end 12 or inlet end portion or upstream end portion and a distal end 14 or outlet end portion or downstream end portion (marked in FIG. 2), and a length therebetween. The prosthetic valve 10 may further include a valve portion, preferably formed by a plurality of prosthetic valve leaflets 16. The valve portion is positioned in a flow channel or passageway for controlling flow through the prosthetic valve 10. The flow channel or passageway is formed by a support structure or valve body 15 of the valve 10. The valve body 15 or support structure has a proximal end portion or inlet end portion or upstream end portion and a distal end portion or outlet end portion or downstream end portion. The prosthetic valve leaflets 16 move between opened and closed states to mimic and replace the operation of native valve leaflets. The leaflets 16 allow flow in a first direction and prevent flow in a second direction. The valve portion is positioned within the passageway of the valve body 15 for permitting flow of blood through the passageway in one direction, thereby replacing the function of a native heart valve. The prosthetic valve leaflets 16 are made of pericardium, such as bovine or porcine pericardium, or another material as desired. In alternative arrangements, the leaflets are formed of a synthetic (e.g., polymer) material or the valve portion is a mechanical one-way valve.

The prosthetic valve leaflets 16 may be coupled to the valve body 15 and may extend radially inward from the valve body 15 into the flow channel. The valve body 15 may surround and support the valve portion and the one or more prosthetic valve leaflets 16.

The valve body 15 may include one or more bodies in examples. The valve body 15, for example may include an inner body 18 (marked in FIG. 2) and an outer body 20.

FIG. 2 illustrates a cross sectional schematic view of the prosthetic valve 10. The inner body 18 may include a proximal portion including a proximal end 22 and a distal portion including a distal end 24. The inner body 18 may have a bulb shape, comprising a curved body that curves radially outward between the proximal end 22 and the distal end 24, or may have another configuration in examples as desired. The inner body 18 may have a circular shape in examples. The inner body 18 may support the plurality of prosthetic valve leaflets 16.

The inner body 18 may include an inner frame 26 (or inner support stent) that may include a plurality of struts 28 (shown in FIG. 4) spaced from each other with spaces 30 (shown in FIG. 4). The plurality of struts 28 form expandable and collapsible cells. Such a configuration may allow the inner frame 26 to move between an undeployed, unexpanded, or linearized configuration to a deployed or expanded configuration. For example, the inner frame 26 may expand radially outward to move to the deployed or expanded configuration, with the length of the inner frame 26 decreasing due to the increased diameter of the inner frame 26. Other configurations of inner frames 26 may be utilized as desired.

The inner frame 26 may include an outer surface 32 or outward facing surface and may include an inner surface 34 or inward facing surface (marked in FIG. 2). The outer surface 32 may face outward from the flow channel 13 and the inner surface 34 may face towards the flow channel 13. The inner frame 26 supports the prosthetic valve leaflets 16. The prosthetic valve leaflets 16 are positioned within an interior of the frame 26.

The inner body 18 may include a skirt 36 (marked in FIG. 2) in examples. The skirt 36 may be positioned on the outer surface 32 of the inner frame 26, or may be positioned on the inner surface 34 of the inner frame 26 (as marked in FIG. 2) in examples. The skirt 36 may extend along the inner frame 26 from the proximal end 22 of the inner body 18 to the distal end 24 of the inner body 18, or may extend along only a portion of the inner frame 26. The skirt 36 may be configured to impede fluid flow therethrough, to impede lateral fluid flow through the flow channel 13 and promote axial flow through the flow channel 13.

In examples, the skirt 36 may be configured for the one or more prosthetic valve leaflets 16 to couple to. For example, a suture line 38 or stitch line between the prosthetic valve leaflets 16 and the skirt 36 may couple the prosthetic valve leaflets 16 to the skirt 36. The suture line 38 or stitch line may have a curved or acuate shape to account for the shape of the prosthetic valve leaflets 16. An outer end portion 40 (marked in FIG. 2) of the prosthetic valve leaflets 16 may couple to the skirt 36, and an opposite inner end portion 42 may be configured for coaptation during opening and closing of the prosthetic valve leaflets 16.

The prosthetic valve leaflets 16 may be coupled to the skirt 36 and may extend radially inward from the skirt 36. The prosthetic valve leaflets 16 may surround the flow channel 13 as marked in FIG. 2 and may move between open and closed states to control flow through the flow channel 13. The proximal end of the prosthetic valve 10 may comprise an inflow end of the valve 10, and the distal end of the prosthetic valve 10 may comprise an outflow end, although other configurations may be utilized as desired. The prosthetic valve leaflets 16 may be positioned around a central axis 43 of the prosthetic valve 10. The inner body 18 and outer body 20 may each surround the central axis 43 of the prosthetic valve 10.

The prosthetic valve 10 may include one or more anchors 44 that may be configured to anchor the prosthetic valve leaflets 16 to a portion of a patient's heart, which may comprise a native valve. The anchors 44 may particularly be configured to anchor to the native valve leaflets of the patient's heart. The anchors 44 may extend around the native valve leaflets to anchor to the native valve leaflets. The anchors 44 may comprise distal anchors positioned at the distal end 14 of the valve 10, or in examples may be provided in another position as desired. The anchors 44 may comprise ventricular anchors extending from a downstream portion of the frame of the prosthetic heart valve and shaped for capturing native leaflets of a native heart valve between the anchors 44 and the outer surface 64 of the outer frame 48.

Each anchor 44 may be configured as a protruding arm configured to extend distally and then curve in a proximal direction to the tip of the respective one of the anchors 44. Such a configuration may allow the anchor 44 to extend around a native leaflet and around the distal tip of the leaflet, to hook over the distal tip of the native valve leaflet and be positioned radially outward of an outward facing surface of a leaflet of the native valve. The anchors 44 may be configured to be in a hooked configuration as shown in FIGS. 1 and 2 for example. The anchors 44 may thus resist a force applied in the atrial or proximal direction to the valve 10 and may anchor the valve 10 within the native valve annulus. Other configurations of anchors 44 may be utilized in examples as desired.

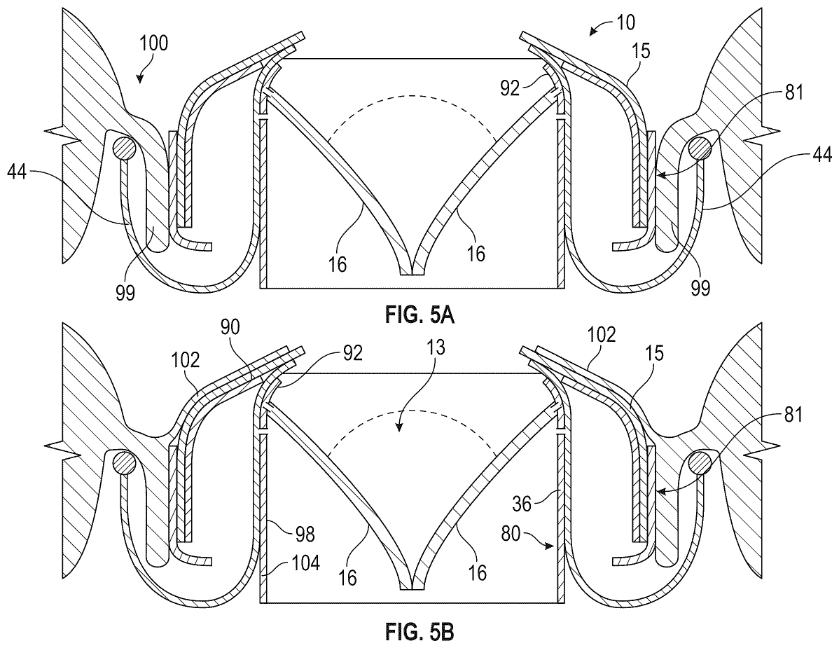

The anchors 44 are shown in FIGS. 1 and 2 in a deployed or expanded configuration, in which the tips of the anchors 44 extend proximally. In examples, the anchors 44 may be configured to be in undeployed, unexpanded, or linearized configuration in which the tips of the anchors 44 extend distally. Such a configuration is represented in FIG. 34 for example. The anchors 44 may be configured to be flexible in examples. Upon deployment, the anchors 44 may be configured to move from the undeployed configuration radially outward to the deployed configuration, with the tips flipped towards the proximal direction. Such an operation may allow the anchors 44 to flip over the native valve leaflets to anchor to the native valve leaflets during deployment. Such a configuration is shown in FIGS. 5A and 5B for example. Other deployment methods for the anchors 44 may be utilized in examples as desired.

The anchors 44 may each extend radially outward from the flow channel 13 and radially outward from the prosthetic valve leaflets 16 of the valve 10. The anchors 44 may be configured to extend radially outward from the inner body 18 and across a gap 46 between the inner frame 26 and an outer frame 48. The anchor 44 may extend to a tip of the respective anchor 44. The anchors 44 may be coupled to a distal end 24 of the inner body 18 and particularly to the distal end of the inner frame 26. The anchors 44 may each include a proximal portion 50 and a distal portion 52, with the proximal portion 50 coupled to the inner frame 26 and the distal portion 52 comprising a tip of the respective anchor 44. The anchors 44 may extend vertically from the proximal portion 50 to the tip at the distal portion 52 when the valve 10 is deployed.

The valve body 15 may include a sealing body 11. The sealing body 11 may be positioned radially outward from the prosthetic valve leaflets 16 and may be configured to seal against a portion of the native valve. The sealing body 11 may comprise the outer surface of the prosthetic valve 10. The sealing body 11 may define the outer diameter of the prosthetic valve 10 and may comprise the outer periphery of the prosthetic valve 10. The sealing body 11 may include a proximal portion having a proximal end 54 and may include a distal portion having a distal end 56 (marked in FIG. 2).

In examples, the sealing body 11 may comprise an outer body 20 that is positioned radially outward of the inner body 18.

Referring to the cross sectional view of FIG. 2, the sealing body 11 may include a frame 48 and a sealing skirt 58, or in examples may comprise only a frame or only a sealing skirt as desired. The frame 48 may comprise an outer frame (or outer support stent) that is positioned radially outward from the inner frame 26.

The outer frame 48 comprises at least a portion of the sealing body 11 that is configured to apply a seal to a portion of a heart. The outer frame 48 may have a proximal portion 60 that couples to the proximal end 22 of the inner body 18. The proximal portion 60 may extend radially outward from the proximal end 22 of the inner body 18 and from the prosthetic valve leaflets 16. A distal portion 62 of the outer frame 48 may be spaced from the prosthetic valve leaflets 16 and the inner frame 26 with the gap 46. The gap 46 may be positioned between the outer frame 48 of the sealing body 11 and a distal portion of the inner body 18.

The outer frame 48 may include an outer surface 64 or outward facing surface and may include an inner surface 66 or inward facing surface. The outward facing surface may face outward from the flow channel 13 and the inward facing surface may face towards the flow channel 13. The outer surface 64 is for pressing against and sealing against tissue of the native heart valve.

In examples, the outer frame 48 may have a length that extends distally to a lesser distance than the distal end of the inner frame 26. As such, the outer frame 48 may be shorter than the inner frame 26. The outer frame 48 may further have a curved configuration that curves outward from the inner frame 26, with a greatest diameter of the outer frame 48 being at the distal portion of the outer frame 48.

The outer frame 48 of the sealing body 11 may include a plurality of struts 68 (as marked in FIG. 1) forming the frame 48, with spaces 70 between the struts. The plurality of struts 68 form expandable and collapsible cells. Such a configuration utilized with the frame 48 may allow the frame 48 to move between an undeployed, unexpanded, or linearized configuration to a deployed or expanded configuration as shown in FIG. 1, in which the outer frame 48 and sealing body 11 have a curved bulbous shape. As with the inner frame 26, the length of the outer frame 48 of the sealing body 11 may decrease as the diameter of the outer frame 48 of the sealing body 11 increases during deployment. The diameter of the outer frame 48 of the sealing body 11 may radially expand outward from the inner frame 26 simultaneously, or at a different time or rate of expansion than the inner frame 26 in examples. In examples, the outer frame 48 may be more flexible than the inner frame 26 and may be adapted to conform to a shape of the native heart valve. The outer frame 48 and inner frame 26 may both be self-expanding, and may be made from a shape memory material. The shape memory material may comprise nitinol or may comprise another material in examples. The self-expanding frame is sized for deployment within the native heart valve. In examples, other forms of expandable frames (e.g., balloon expandable or mechanically expandable) may be utilized.

The sealing body 11 may include the sealing skirt 58, which may be coupled to the outer frame 48 of the sealing body 11 or may be free from the outer frame 48 in examples. The sealing skirt 58 may extend along the outer frame 48. The sealing skirt 58 may comprise a fabric skirt that covers at least a portion of the outer surface 64 of the outer frame 48.

The sealing skirt 58 may have a distal portion 72 and a proximal portion 74 in examples. The distal portion 72 may extend over a distal portion of the outer frame 48, and may extend over the outer surface 64 of the outer frame 48 for example. The distal portion 72 may extend proximally to an end 76, which may be positioned at a midpoint of the outer frame 48, for example, or at another position as desired. The distal portion 72 may extend distally to couple to the inner body 18, by spanning the gap 46 for example. In examples, the distal portion 72 may couple to the one or more anchors 44.

The proximal portion 74 of the sealing skirt 58 may be positioned upon an inner surface 66 or inward facing surface of the outer frame 48. The proximal portion 74 of the sealing skirt 58 may extend proximally to the proximal end 54 of the sealing body 11 in examples.

The sealing skirt 58 may be made of a material that resists fluid flow therethrough, such as a cloth material, woven material, or other material such as a polymer or other material that resists fluid flow therethrough. The material may comprise a fabric. A variety of materials may be utilized for the skirt 58 as desired.

The sealing body 11 may be configured to abut a portion of the patient's heart to reduce fluid flow. The skirt 58 may be configured to seal a portion of the native valve annulus. For example, the sealing body 11 may abut a surface of a patient's native valve leaflet to reduce fluid flow between the sealing body 11 and the native leaflet. The sealing body 11 may be configured to abut other portions of the patient's heart to reduce fluid flow as desired.

The sealing body 11 may be flexible to allow for movement and conformability to a native valve annulus.

In examples, at least a portion of the prosthetic valve 10 may include a material that may be configured to reduce tissue formation or thrombus formation along the portion of the prosthetic valve 10. The material may have a variety of forms. For example, the material may comprise a textile that is configured to reduce tissue formation or thrombus formation with the textile. The textile may be configured to be thromboresistant to reduce the possibility of thrombus formation. The textile may otherwise be configured to reduce tissue formation or thrombus formation. The material may comprise a coating on a portion of the prosthetic valve 10. The coating may be thromboresistant or otherwise configured to reduce tissue formation or thrombus formation. The coating may be provided on a frame, a skirt, or on another portion of the prosthetic valve 10 as desired. In examples, the material may comprise the material that forms the portion of the prosthetic valve. For example, the frame may be constructed from a material that resists tissue formation or thrombus formation. One or more of the prosthetic valve leaflets may be constructed from a material that resists tissue formation or thrombus formation. Any portion of the prosthetic valve may be configured to resist tissue formation or thrombus formation. The tissue formation may comprise pannus formation in examples. Other forms of tissue ingrowth may be reduced.

All or a portion of a skirt may comprise the material configured to reduce tissue formation or thrombus formation. A skirt may be constructed from a material (e.g., textile) that resists tissue formation or thrombus formation or otherwise may be coated with a material that resists tissue formation or thrombus formation. The skirt may be positioned at a variety of locations. For example, all or a portion of an outer skirt or sealing skirt 58 may comprise the material configured to reduce tissue formation or thrombus formation. In examples, all or a portion of the skirt 36 of the inner body 18 may comprise the material configured to reduce tissue formation or thrombus formation.

An inner surface 80 of the valve body 15 that faces towards the flow channel 13 may comprise the portion of the prosthetic valve 10 that may be configured to reduce tissue formation or thrombus formation. The inner surface 80 may face opposite the outer surface 81 of the valve body 15, which may comprise the outer surface 64 of the outer frame 48 or the outer surface of the sealing skirt 58 and may face outward from the flow channel 13. The inner surface 80 may bound the flow channel 13.

In examples, an end portion of the prosthetic valve 10 may comprise the portion of the prosthetic valve 10 that may be configured to reduce tissue formation or thrombus formation. For example, a proximal end portion 82 of the prosthetic valve 10 may comprise the portion of the prosthetic valve 10 that may be configured to reduce tissue formation or thrombus formation, although a distal end portion 84 of the prosthetic valve 10 may be utilized alternatively or in combination. The proximal end portion 82 may comprise an inflow of the prosthetic valve 10, and the distal end portion 84 may comprise an outflow of the prosthetic valve 10, although other configurations may be utilized as desired.

The portion of the prosthetic valve 10 that may be configured to reduce tissue formation or thrombus formation may comprise one or more of the proximal end portion 86 of the inner surface 80 of the valve body 15 or the proximal end portion 88 of the outer surface 81 of the valve body 15. The end portions 86, 88 may be proximate to each other as shown in FIG. 2, for example. The end portions 86, 88 may be coupled to each other in examples. For example, referring to FIG. 1, a proximal end of the sealing skirt 58 may be stitched or otherwise coupled to the proximal end of the skirt 36. The skirts 58, 36 may couple to each other at a proximal rim 90 of the prosthetic valve 10.

In examples, a portion of the prosthetic valve 10 configured to reduce tissue formation or thrombus formation may comprise a barrier to tissue ingrowth at one or more locations. The one or more locations may be at a variety of locations on the prosthetic valve 10 as desired. For example, referring to FIG. 2, all or a portion of the outer skirt 58 or sealing skirt may comprise a barrier to tissue formation or thrombus formation. In examples, all or a portion of the skirt 36 of the inner body 18 may comprise a barrier to tissue formation or thrombus formation. Other portions of the prosthetic valve 10 may comprise a barrier to tissue formation or thrombus formation as desired.

In examples, the barrier may be configured to reduce or prevent tissue formation or thrombus formation along the inner surface 80 of the valve body 15 that faces towards the flow channel 13. The inner surface 80 may extend along the flow channel 13. The prosthetic valve leaflets 16 may protrude radially inward from the inner surface 80.

All or a portion of the skirt 36 may comprise a barrier layer that may reduce tissue formation or thrombus formation. The barrier layer may be configured to be positioned adjacent to the prosthetic valve leaflets 16.

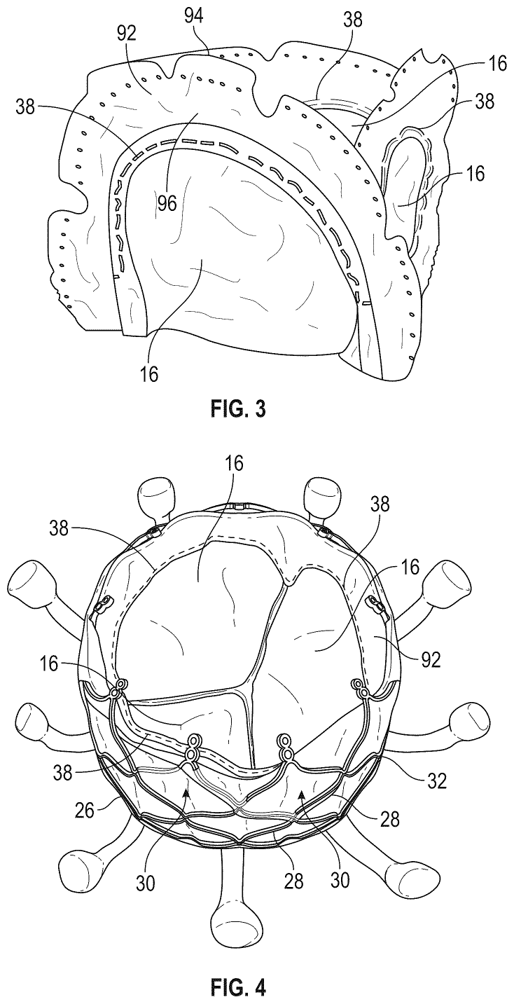

The barrier layer may comprise a band 92 of material. FIG. 3, for example, illustrates a band 92 of material comprising a barrier layer. The band 92 may have a variety of shapes, and may have an arcuate shape as shown in FIG. 3 or another shape as desired. The band 92 may be configured for the prosthetic valve leaflets 16 to couple to. The prosthetic valve leaflets 16, for example, may couple to the barrier layer or band 92 with the suture line 38 or stitch line. The band 92 may extend proximally from the suture line 38 or stitch line to a proximal end portion 94 of the band 92. The band 92 may form a portion of the skirt 36 that may be positioned along the inner frame 26 and may line the inner surface 34 of the inner frame 26.

FIG. 4, for example, illustrates a position of the band 92 relative to the inner frame 26. The band 92 may be positioned at the proximal end portion 86 of the inner surface 80 of the valve body 15 and may extend distally from the proximal end portion 86. The prosthetic valve leaflets 16 may be coupled to the barrier layer or band 92 and may extend distally from the suture line 38 or stitch line with the barrier layer or band 92.

In examples, the barrier layer or band 92 may couple to a portion of the sealing skirt 58 of the sealing body 11. The proximal end portion 94 of the band 92, for example, may couple to the proximal portion 74 of the sealing skirt 58. FIG. 1 illustrates that the proximal end portion 94 of the band 92 may join with the proximal portion 74 of the sealing skirt 58. A suture line or stitch line may be provided to couple the proximal end portion 94 of the band 92 to the proximal portion 74 of the sealing skirt 58, or other forms of coupling may be utilized in examples.

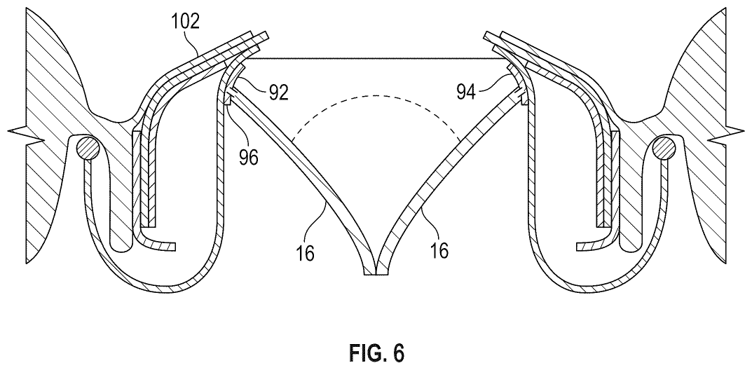

In examples, a distal end portion 96 of the barrier layer or band 92 (marked in FIG. 3) may couple to a proximal end portion of a remainder 98 of the skirt 36 (marked in FIG. 2). The remainder 98 of the skirt 36 may extend along and bound a portion of the flow channel 13 distal of the connection with the prosthetic valve leaflets 16. The band 92 and the remainder 98 of the skirt 36, alternatively or in combination, may form a sleeve bounding the flow channel 13. In examples, the remainder 98 of the skirt 36 may be excluded, as shown in FIG. 6 for example.

The barrier layer or band 92 may be configured to reduce the spread of tissue formation or thrombus formation to the one or more prosthetic valve leaflets 16. For example, referring to FIG. 5A, the prosthetic valve 10 is illustrated in an implanted configuration in which the anchors 44 are anchored to native valve leaflets 99 of the native valve 100. The outer surface 81 of the valve body 15 may be in contact with the native valve leaflets 99 or another portion of the native valve 100.

Over time, following implantation, tissue formation or thrombus formation may occur along a portion of the prosthetic valve 10. For example, referring to FIG. 5B, tissue formation 102 or thrombus formation, which may be in the form of pannus growth has occurred along a surface of the prosthetic valve 10. For example, an outer surface 81 of the valve body 15 experiences the tissue formation 102. The tissue formation 102 may serve to implant and further secure the prosthetic valve 10 to the implantation site in examples. The barrier layer or band 92, however, may serve to reduce tissue formation or thrombus formation. The barrier layer or band 92 may impede tissue formation or thrombus formation along the inner surface 80 of the valve body 15 and to the prosthetic valve leaflets 16. The barrier layer or band 92, for example, may impede distal tissue formation or thrombus formation from the proximal rim 90 of the prosthetic valve 10. As such, a reduced possibility of tissue formation or thrombus formation to the prosthetic valve leaflets 16 may result.

Such a result may beneficially reduce the possibility of an impediment to the operation of the prosthetic valve leaflets 16 due to tissue formation or thrombus formation along the prosthetic valve leaflets 16. Further, improved flow along the flow channel 13 may result due to a reduced possibility of tissue formation or thrombus formation along the flow channel 13. A reduced possibility of valve stenosis or failure may result.

In examples, a distal end portion 104 of the skirt 36 may be configured to reduce tissue formation or thrombus formation. The distal end portion 104, for example, may reduce the possibility of tissue formation or thrombus formation in a proximal direction towards the prosthetic valve leaflets 16. In examples, and referring to FIG. 6, the remainder 98 of the skirt 36, which may comprise the distal end portion 104 of the skirt 36, may be excluded and the band 92 (as marked in FIG. 3) may comprise the entirety of the barrier layer for the prosthetic valve leaflets 16.

In examples, other portions of the prosthetic valve 10 may include a material that may be configured to reduce tissue ingrowth along the portion of the prosthetic valve 10 as desired. In examples, portions of the prosthetic valve 10 may be configured for tissue ingrowth. For example, an outer surface 81 of the valve body 15 may be configured to tissue ingrowth for purposes of sealing or anchoring with an implantation site. Another portion of the prosthetic valve 10, however, such as a barrier layer or band 92, or other portion of the prosthetic valve 10 may reduce tissue ingrowth as desired.

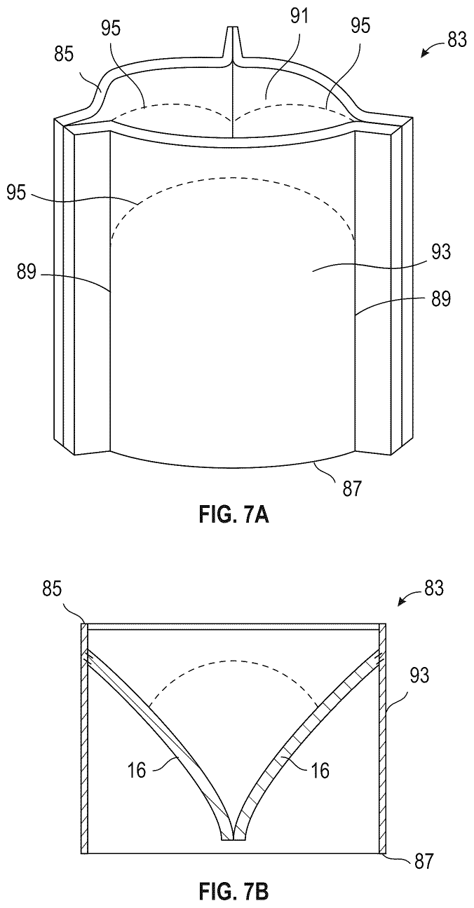

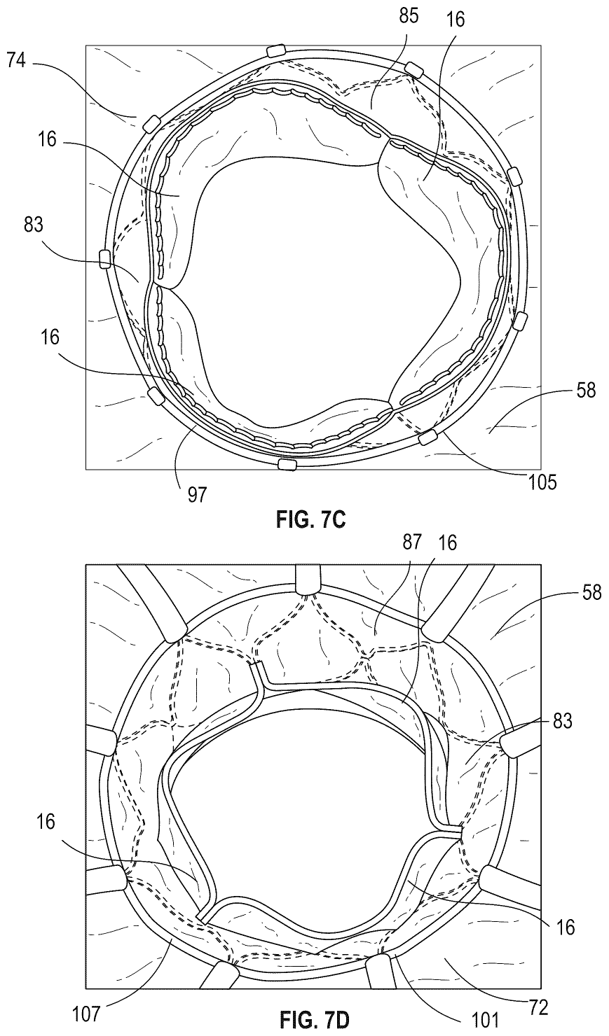

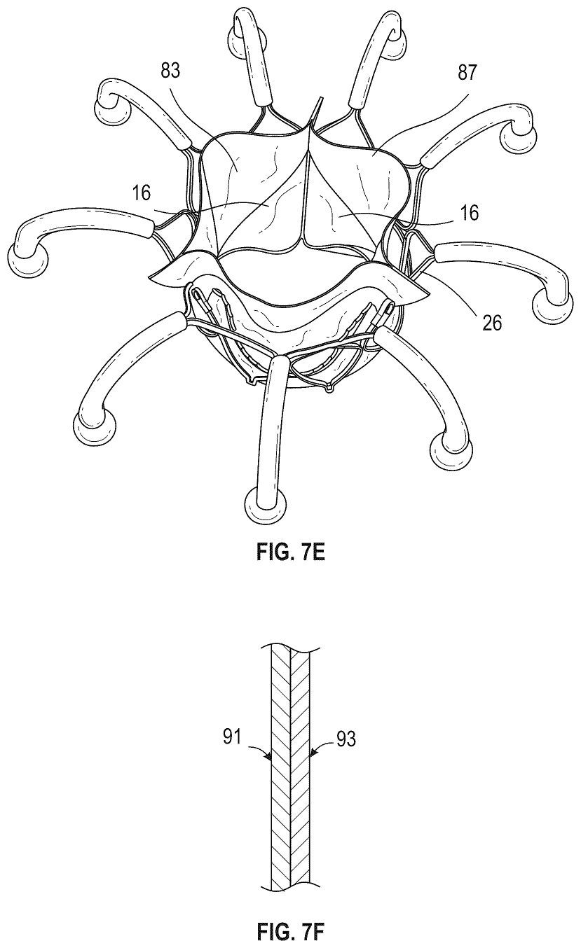

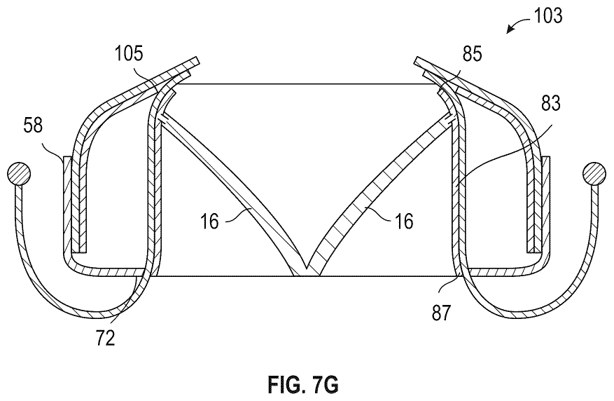

FIG. 7A illustrates a perspective view of an example in which a skirt 83 may be provided that may include similar features as the skirt 36 unless stated otherwise. The skirt 83 may extend from a proximal end portion 85 to a distal end portion 87. The skirt 83 may comprise a sleeve configured to be positioned interior of the inner frame 26. The skirt 83 may extend along the inner surface 34 or inward facing surface of the inner frame 26. The skirt 83 may comprise multiple portions of material sutured or stitched together along stitch lines 89.

One or more of the prosthetic valve leaflets 16 may extend radially inward from an inner surface 91 of the skirt 83. The inner surface 91 may face towards the flow channel and may face opposite the outer surface 93 of the skirt 83. The one or more of the prosthetic valve leaflets 16 may be coupled to the skirt 83 with a suture line or stitch line 95.

FIG. 7B illustrates a cross sectional view of the skirt 83.

Referring to FIG. 7C, the proximal end portion 85 of the skirt 83 may be configured to couple to the proximal portion 74 of the sealing skirt 58. The proximal end portion 85 of the skirt 83, for example, may couple to the proximal portion 74 of the sealing skirt 58 along a stitch line 97. The connection of the inner skirt 83 to the sealing skirt 58 may comprise a proximal rim 105 of the prosthetic valve.

Referring to FIG. 7D, the distal end portion 87 of the skirt 83 may be configured to couple to the distal portion 72 of the sealing skirt 58. The distal end portion 87 of the skirt 83, for example, may couple to the distal portion 72 of the sealing skirt 58 along a stitch line 101. The connection of the inner skirt 83 to the sealing skirt 58 may comprise a distal rim 107 of the prosthetic valve.

FIG. 7E illustrates a position of the skirt 83 relative to the inner frame 26 prior to being coupled to the sealing skirt 58.

The skirt 83 may be configured to reduce tissue formation or thrombus formation along the length of the skirt 83. An outer or inner surface of the skirt 83 may be configured to reduce tissue formation or thrombus formation along the length of the skirt 83. The skirt 83 may comprise a barrier layer for reducing tissue formation or thrombus formation towards the prosthetic valve leaflets 16 and along the flow channel of the prosthetic valve. The distal end portion 87 of the skirt 83 and the proximal end portion 85 of the skirt 83 may each be configured to reduce tissue formation or thrombus formation in a respective proximal or distal direction towards the leaflets 16. The distal end portion 87 of the skirt 83 and the proximal end portion 85 of the skirt 83 may each be configured to reduce tissue formation or thrombus formation from the respective distal rim 107 or proximal rim 105.

In examples, a surface of the skirt 83 may include a coating or layer that may reduce tissue formation or thrombus formation. For example, referring to FIG. 7F, the inner surface 91 of the skirt 83 may comprise a coating or layer that may reduce tissue formation or thrombus formation. The inner surface 91 may comprise a polymeric or laminate material that may reduce tissue formation or thrombus formation. The outer surface 93 of the skirt 83 may comprise a textile or other form of material. In examples, both surfaces 91, 93 of the skirt 83 may reduce tissue formation or thrombus formation.

FIG. 7G illustrates a prosthetic valve 103 utilizing the skirt 83. The distal end portion 87 of the skirt 83 may be configured to couple to the distal portion 72 of the sealing skirt 58. The prosthetic valve 103 may include the features of the prosthetic valve 10 unless stated otherwise.

The features of FIGS. 1-7G may be utilized solely or in combination with any other example disclosed herein.

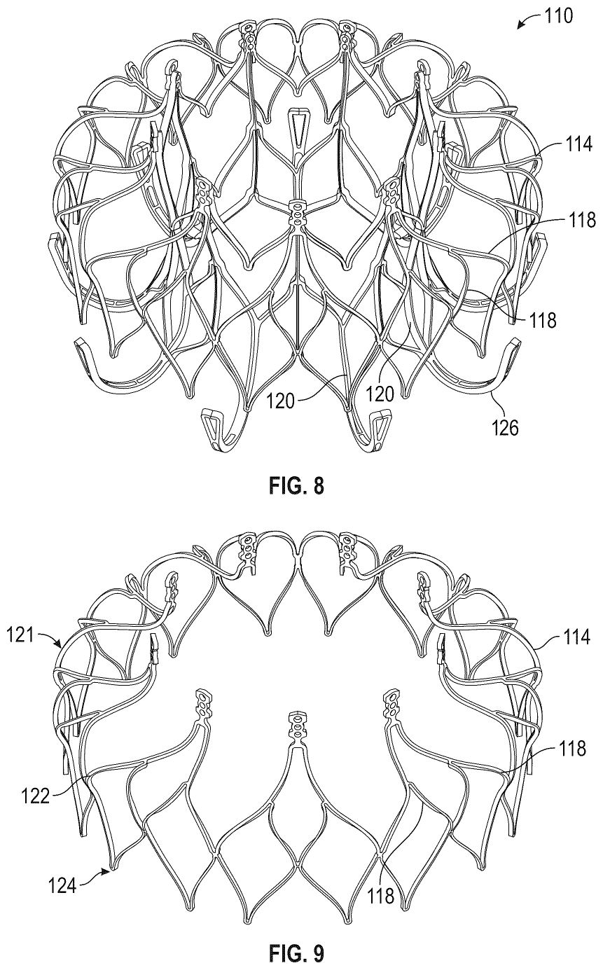

In examples, other forms of prosthetic valves may be utilized. FIG. 8, for example, illustrates a configuration of a frame 110 of a prosthetic valve 112 (marked in FIG. 11). The frame 110 and prosthetic valve 112 may include the features of the frame and prosthetic valve 10 discussed in regard to FIGS. 1-7G unless stated otherwise. The frame 110, for example, may include an outer frame 114 and an inner frame 116. The outer frame 114 may include a plurality of struts 118 separated by spaces and forming a lattice, similar to the outer frame 48 of FIG. 1. The inner frame 116 may similarly include a plurality of struts 120 separated by spaces and forming a lattice, similar to the inner frame 26 of FIG. 4.

FIG. 9 illustrates the outer frame 114 separate from the inner frame 116. The outer frame 114 may be configured to form a shoulder 121 or protrusion of the outer surface 122 of the outer frame 114 relative to a distal end portion 124 of the outer frame 114. The shoulder 121 may serve to reduce distal or ventricular movement of the prosthetic valve 112 upon implantation.

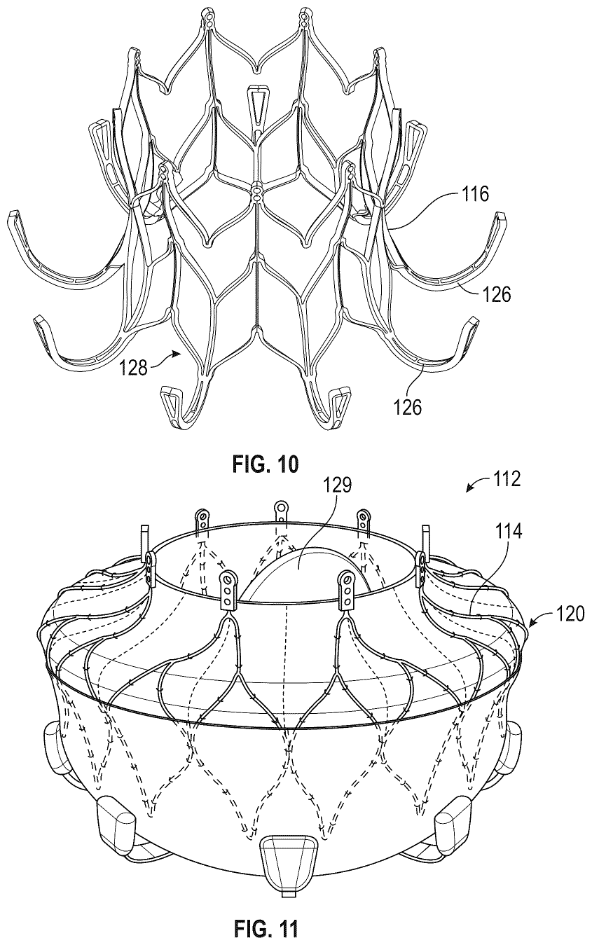

FIG. 10 illustrates the inner frame 116 separate from the outer frame 114. A plurality of arms 126 comprising anchors may extend radially outward from a distal end portion 128 of the inner frame 116.

FIG. 11 illustrates a perspective view of the prosthetic valve 112 with one or more skirts applied to the frames 114, 116. The prosthetic valve leaflets 129 are positioned within the flow channel of the prosthetic valve 112.

Features of the prosthetic valve 112 may be utilized with any example disclosed herein.

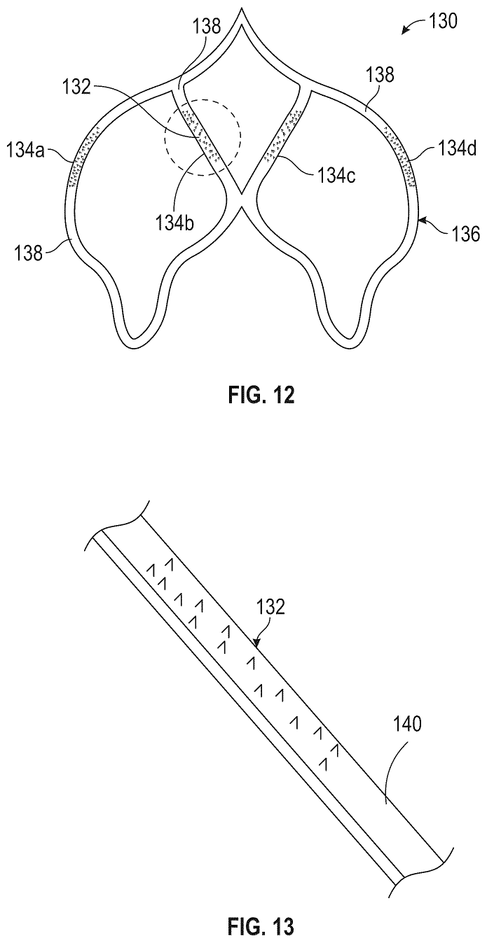

In examples, at least a portion of a prosthetic valve may include a frame having a rough surface. Referring to FIG. 12, for example, a portion of a frame 130 may include a rough surface 132. The portion of the frame 130 may include struts 134a-d, which may be configured and utilized in a similar manner as with other frames disclosed herein. The rough surface 132 may comprise all or a portion of the frame 130.

The rough surface 132 may be positioned on an outer surface 136 of the frame 130 in examples. The outer surface 136, for example, may be configured for contact with a portion of a native valve such as a native valve leaflet or annulus or other portion. The rough surface 132 may be configured to provide friction with a portion of a native valve. Such friction may be utilized to improve anchoring with the portion of the native valve. In examples, the frame 130 may comprise an outer frame or portion of a sealing body of a prosthetic valve for forming a seal with a native valve. The outer frame or portion of the sealing body may be configured for contact with a portion of a native valve. For example, referring to FIG. 1, a proximal portion of the outer frame 48 is exposed and may contact a portion of a native valve. Referring to FIG. 11, a proximal portion of the outer frame 114 may be similarly exposed and may contact a portion of a native valve. In examples, the rough surface 132 may be positioned at a distal portion of an outer frame and may be configured for contact with a portion of a native valve.

In examples, other portions of a frame may include a rough surface. For example, one or more of the anchors (e.g., distal anchors) may include the rough surface. Such a surface may increase friction with one or more of the native valve leaflets to improve anchoring. All or a portion of an inner frame may include a rough surface. Such a surface may be utilized for coupling to prosthetic valve leaflets or for providing another function.

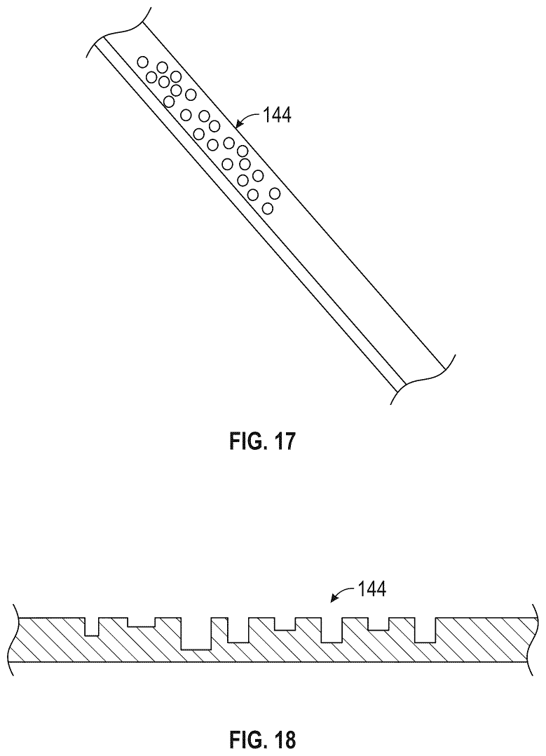

The rough surface may have a variety of forms. In examples, the rough surface may comprise a pattern of protrusions extending from the surface of the frame 130. In examples, the rough surface may comprise one or more voids in the surface of the frame 130 (as represented in FIGS. 17 and 18 for example).

In examples, portions 138 of the frame 130 adjacent to the rough surface 132 may be smooth. One or more rough surfaces 132 may be selectively positioned on portions of the frame 130 to provide a desired configuration of the rough surfaces 132. One or more smooth portions 138 may be positioned adjacent as desired.

In examples, the smooth portions 138 may comprise polished surfaces of the frame 130. The rough surfaces 132 may comprise unpolished surfaces forming the surface roughness. The polishing may occur during a formation process of the frame 130. Areas of the frame 130 may be selectively polished for forming portions of the frame having the surface roughness and portions that are smooth.

In examples, the rough surfaces 132 may comprise a residue of a formation process of the frame. For example, as the frame is formed, a surface residue may result that may normally be removed prior to use of the frame. However, the surface residue may remain and may be left unpolished in one or more areas to remain on the surface of the frame. In examples, the frame may be made of a shape memory material (such as nitinol or another form of shape memory material) and the surface residue may be present on the shape memory material. The rough surfaces may comprise an unpolished surface roughness of the nitinol.

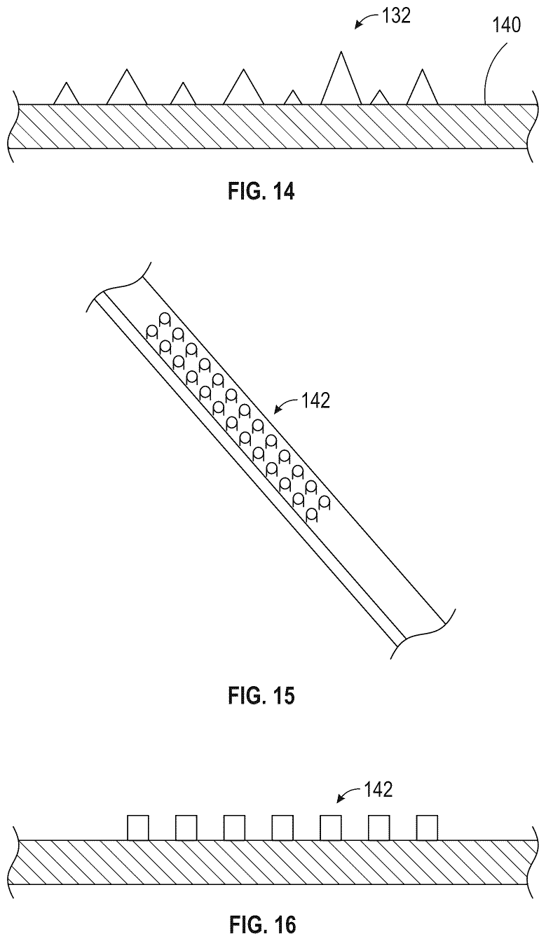

The pattern of the rough surface 132 may be irregular in examples. For example, FIG. 13 illustrates a close up view of the rough surface 132 having an irregular pattern. The irregular pattern may result from the irregular nature in which the rough surface 132 comprises residue from a formation process. FIG. 14 illustrates a side view of the rough surface 132 shown in FIG. 13. The rough surface 132 may include a plurality of peaks that may have irregular spacing and height from the base surface 140 of the frame 130.

In examples, a rough surface may be formed in a variety of other manners, such as sputtering or deposition of particulate matter upon the frame, or through physical abrasion of the frame to form a rough surface.

In examples, a microstructure may be formed on a frame. For example, FIG. 15 illustrates a microstructure 142 forming a regular pattern on the frame. The microstructure may be configured to produce a Van der Waals force interaction to provide adhesion to an implantation site. FIG. 16 illustrates a side view of the microstructure 142. Other forms of friction may be provided due to a regular pattern. A microstructure 142 may have a variety of forms including circles, squares, points, triangles, or others.

In examples, the rough surface may comprise one or more voids 144 in a surface of a frame. FIG. 17, for example, illustrates a plurality of voids 144, with FIG. 18 providing a cross sectional side view of the voids 144. The voids 144 may be irregular or regular in configuration. The voids 144 may be formed in a variety of manners including chemical erosion or physical impact against the surface of the frame.

In examples, a rough surface may be utilized for friction with a portion of a native valve. A rough surface may improve anchoring or fixation of a prosthetic valve to an implantation site. In examples, the rough surface may be utilized for tissue ingrowth. For example, a rough surface may provide improved growth of tissue for adhesion and sealing as desired. Tissue formation or thrombus formation may result. A series of voids 144 for example, or a series of protrusions, may provide for improved tissue growth. The rough surface may be utilized for other purposes in examples.

The features of FIGS. 12-18 may be utilized solely or in combination with any other example disclosed herein.

In examples, a valve body may include one or more grip features configured to be positioned radially inward of one or more of the native valve leaflets. One or more anchors may be configured to be positioned radially outward of the one or more native valve leaflets and press the one or more native valve leaflets towards the one or more grip features to reduce movement of the one or more of the native valve leaflets relative to the valve body. The anchors may press the native valve leaflets against the grip features for securing the prosthetic heart valve within the native heart valve.

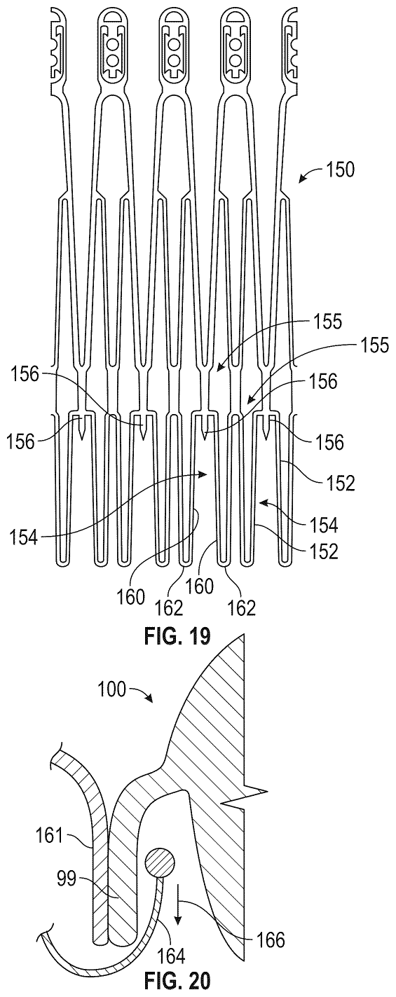

For example, referring to FIG. 19, a frame 150 of a sealing body is shown in a flat pattern. The frame 150 may include features of the outer frame 114 shown in FIG. 9, or may include features of the outer frame 48 shown in FIG. 1 unless stated otherwise. The frame 150 may comprise a portion of a sealing body for forming a seal with a native valve or may be otherwise configured. The frame 150 may comprise an outer frame of a multi-frame prosthetic valve or may be utilized in a single frame implementation.

The frame 150 may include a plurality of struts 152 and openings 154, 155 between the struts, similar to other frames disclosed herein.

In examples, one or more grip features 156 may be provided. The grip features 156 may have a variety of forms. For example, referring to FIG. 19, the grip features 156 may comprise one or more protrusions extending from the frame 150. Each protrusion, for example, may extend distally. Other directions of protrusion may be utilized in examples.

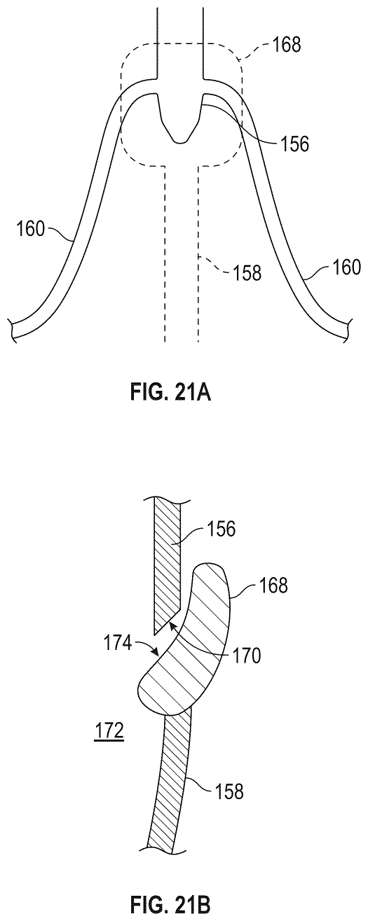

The protrusions may be positioned to circumferentially align with an anchor of the prosthetic valve in examples. For example, referring to FIG. 21, an anchor 158 is shown to align with the grip feature 156, with the tip 168 of the anchor 158 positioned at the grip feature 156. Other positions of grip features may be utilized in examples.

Referring to FIG. 19, in examples, the grip features 156 may extend distally towards an opening 154 that the anchors 158 may be circumferentially aligned with. The opening 154 may be positioned between adjacent struts 160 that may extend distally from a grip feature 156. The adjacent struts 160 may extend distally to an apex 162 of the struts 160 that may comprise the distalmost portion of the frame 150. The grip features 156 may be positioned at the proximal end portions of the struts 160.

The grip features 156 may each include a pointed tip, and may comprise a prong or barb in examples. The grip features 156 may have other configurations in examples.

The anchors 158 may be configured to press the one or more native valve leaflets towards the one or more grip features to reduce movement of the one or more of the native valve leaflets relative to the valve body. The anchors 158 may be configured to clamp with the one or more grip features. An anchor 158 may be biased to exert an inward force against a leaflet to press the leaflet towards a grip feature. A grip feature or valve body may be biased to exert an outward force against a leaflet for the anchor 158 to press the leaflet towards a grip feature. In examples, the anchor and valve body may not be biased, but may be sized and positioned relative to each other for the anchor to press the one or more native valve leaflets towards the one or more grip features. For example, FIG. 20 illustrates a configuration of a valve body 161 and anchor 164 excluding use of grip features. The native valve leaflet 99 may possibly slide proximally relative to the valve body 161 following implantation. For example, the native valve 100 may dilate or otherwise vary the configuration of the native valve leaflet 99 or of the native valve 100. The valve body 161 may slide distally (represented by the arrow 166) which may be undesirable. The use of anchors 158 configured to press the one or more native valve leaflets towards the one or more grip features to reduce movement of the one or more of the native valve leaflets relative to the valve body may beneficially reduce the possibility of such axial movement of the one or more of the native valve leaflets relative to the valve body. Distal and ventricular movement of the prosthetic valve may be resisted. Adverse results from dynamic fluctuations in valve anatomy (e.g., dilation) may be reduced.

FIG. 21A illustrates a plan view of the position of the anchor 158 relative to the grip feature 156. The anchor 158 may include a tip 168 that may align with the position of the grip feature 156. The tip 168 of the anchor 158 may be at the same circumferential position as the grip feature 156 and may be at the same axial position or height as the grip feature 156. A native valve leaflet accordingly may be pressed between the grip feature 156 and the tip 168 of the anchor 158.

FIG. 21B, for example, illustrates a side cross sectional view of the configuration shown in FIG. 21A. The tip 168 is positioned to press a native valve leaflet in the space 170 between the tip 168 and the grip feature 156.

In examples, the anchor 158 may be configured to press a native valve leaflet towards a grip feature 156 to deflect a native valve leaflet between the grip feature 156 and the anchor 158. For example, the anchor 158 and the grip feature 156 may be positioned such that a leaflet may deflect radially inward towards an area 172 radially inward of the grip feature 156 and the anchor 158. Such deflection may further enhance a grip of the native valve leaflet.

In examples, a portion of an anchor 158 may include a recess configured to receive a grip feature 156. Referring to FIG. 21B, for example, the tip 168 of the anchor 158 includes the recess 174 in the form of a contoured surface of the tip 168. The contoured surface may accommodate the shape of the grip feature 156, and may aid in the deflection of the native valve leaflet radially inward. Other configurations of anchors 158 and grip features 156 may be provided in examples.

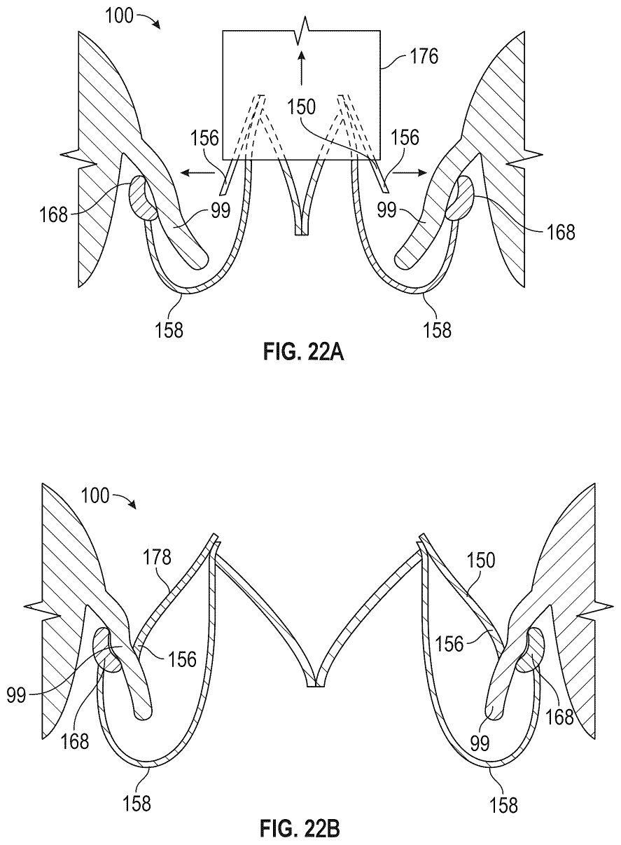

FIGS. 22A and 22B illustrate an exemplary deployment of a prosthetic valve including the grip features 156 and the anchors 158. For example, referring to FIG. 22A, the anchors 158 are shown to be deployed to the native valve 100 with the tips 168 of the anchors 158 positioned radially outward of the native valve leaflets 99. The frame 150 may expand radially outward and approach the native valve leaflets 99. A retaining body such as a capsule 176 may be retracted to allow the frame 150 to expand.

FIG. 22B illustrates the frame 150 having been expanded. The anchors 158 press the one or more native valve leaflets 99 towards the one or more grip features 156 to reduce movement of the one or more of the native valve leaflets 99 relative to the valve body 178. The possibility for distal or ventricular movement of the valve body 178 relative to the native valve leaflets 99 may be reduced. The native valve leaflets 99 are further shown to deflect between the grip features 156 and the anchors 158.

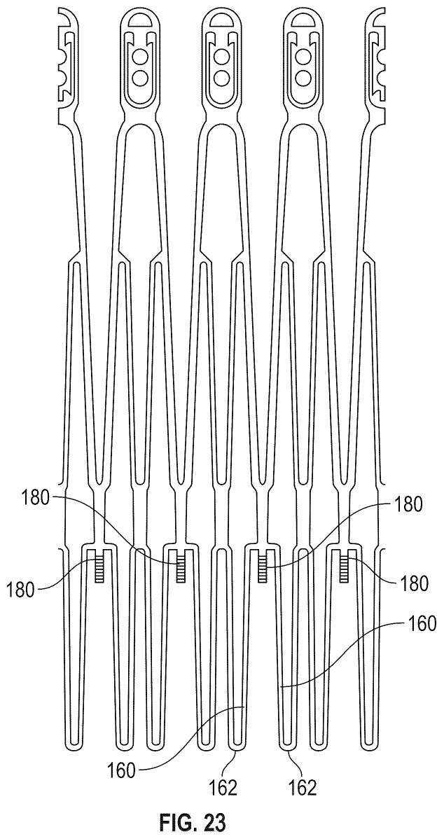

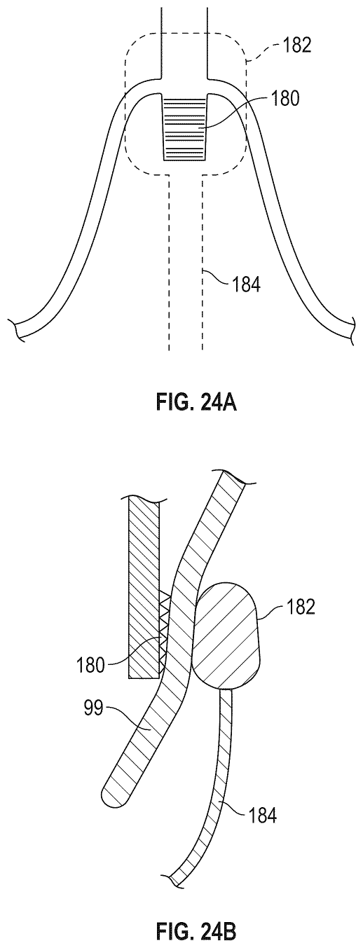

In examples, the configuration of the grip features or the anchors may be varied as desired. For example, referring to FIG. 23, grip features 180 may comprise a surface roughness of a frame. The surface roughness may be formed in a variety of manners as desired. The grip features 180 may comprise protrusions that the surface roughness may be positioned upon. The protrusions may be positioned in a similar location as the grip features 156 shown in FIG. 19, or may be provided in a different location as desired. The grip features 180 may align with an anchor 184 (marked in FIG. 24A) in a similar manner as the grip features 156 shown in FIG. 19. For example, FIG. 24A illustrates an alignment of the grip features 180 with a tip 182 of an anchor 184.

FIG. 24B illustrates a side cross sectional view of the position of the grip feature 180 relative to the anchor 184. The grip feature 180 contacts the native valve leaflet 99 to provide friction against the native valve leaflet 99. The grip feature 180 clamps with the anchor 184.

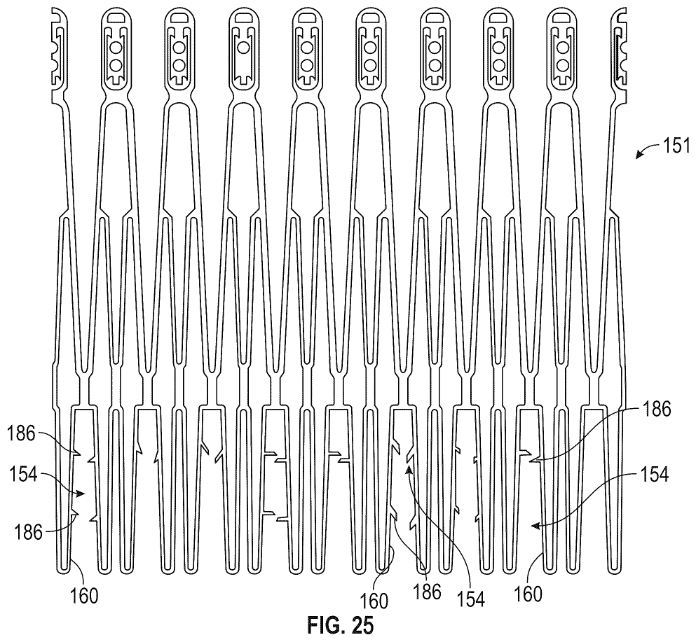

Other forms of grip features or anchors may be provided in examples. For example, FIG. 25 illustrates an example in which one or more grip features 186 are positioned on the longitudinally extending struts 160 of the frame 151. The grip features 186 are provided along the outer surface of the frame 151. The struts 160 bound the openings 154 that the anchors are circumferentially aligned with. The grip features 186 comprise protrusions that extend laterally inward towards the openings 154. The protrusions may comprise prongs or barbs. A plurality of the grip features 186 may extend laterally inward towards a respective one of the openings 154. Other locations, e.g., extending from the apices of the struts 160, may be utilized. The grip features may be in an asymmetric arrangement in examples. The grip features may point distally, or in an outflow or distal direction, or may point proximally or in an inflow or proximal direction. The grip features may flare radially outwards in examples. In examples, the grip features may be positioned only along a downstream, outflow, or distal portion of the frame 151 as represented in FIG. 25. In examples, the grip features may be positioned along the entirety of the outer frame.

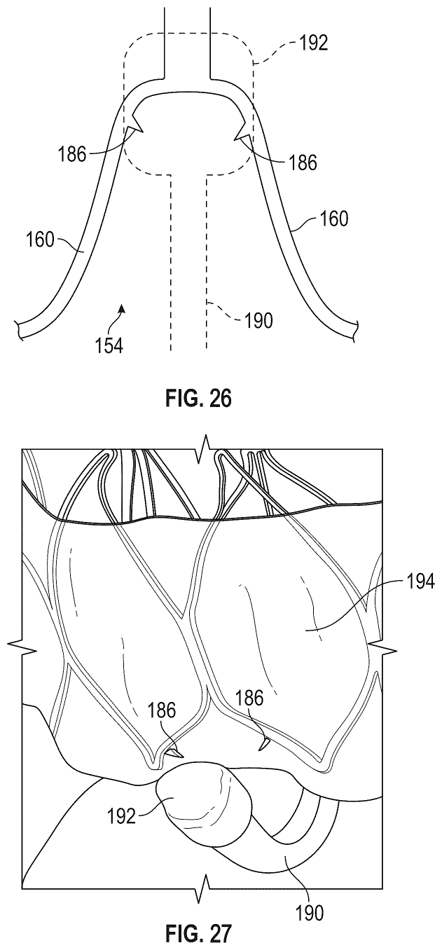

The anchors 190 may circumferentially align with the openings 154. For example, FIG. 26 illustrates an example of the position of the anchors 190 relative to the opening 154 of the frame 151. The tip 192 of the anchor 190 is shown to be circumferentially aligned with the opening 154 of the frame 151. FIG. 27 illustrates a perspective view of the anchor 190 relative to the grip features 186.

The grip features disclosed herein may protrude from a skirt or other material of a prosthetic valve. For example, as shown in FIG. 27, the grip features 186 protrude from or extend through the sealing skirt 194 of the prosthetic valve. In examples, the grip features may protrude from a skirt, or may be covered with a skirt as desired.

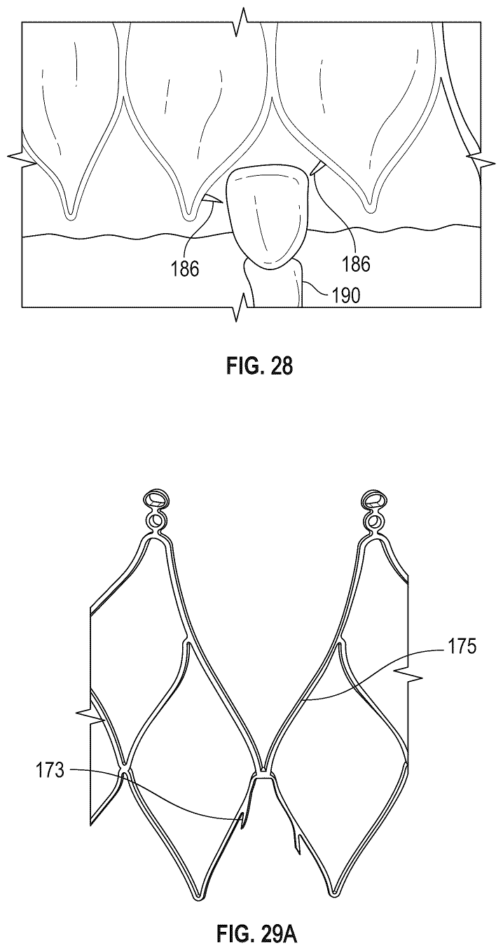

The position of grip features may be varied in examples such that the grip features are positioned offset from a position of an anchor. For example, FIG. 28 illustrates a variation in which the grip features 186 are circumferentially offset from the anchor 190. The circumferential offset may be increased from the configuration that is shown in FIG. 27 as desired. The anchor 190 may be configured to clamp with the one or more grip features that are circumferentially offset from the position of the anchor 190.

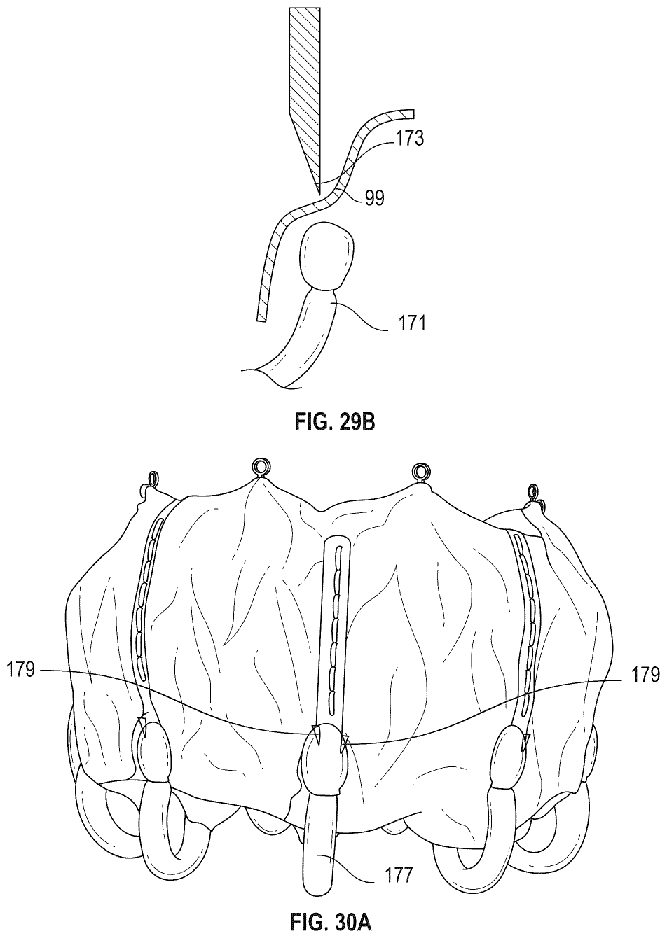

FIGS. 29A and 29B illustrate an example in which the anchor 171 may be positioned at a same radial distance as a grip feature 173 upon clamping with the grip feature 173. The tip of the anchor 171 may be positioned distal of the grip feature 173. The anchor 171 may press the native valve leaflet 99 towards the grip feature 173 to reduce movement of the native valve leaflet 99 relative to the valve body. The anchor 171 may deflect the native valve leaflet 99 radially inward, as shown in FIG. 29B.

The grip features 173 may be positioned on struts of the frame 175 as shown in FIG. 29A for example. The grip features 173 may be positioned in a similar manner as the grip features shown in FIGS. 25-28. In examples, the grip features 173 may be positioned in other locations or may have other configurations.

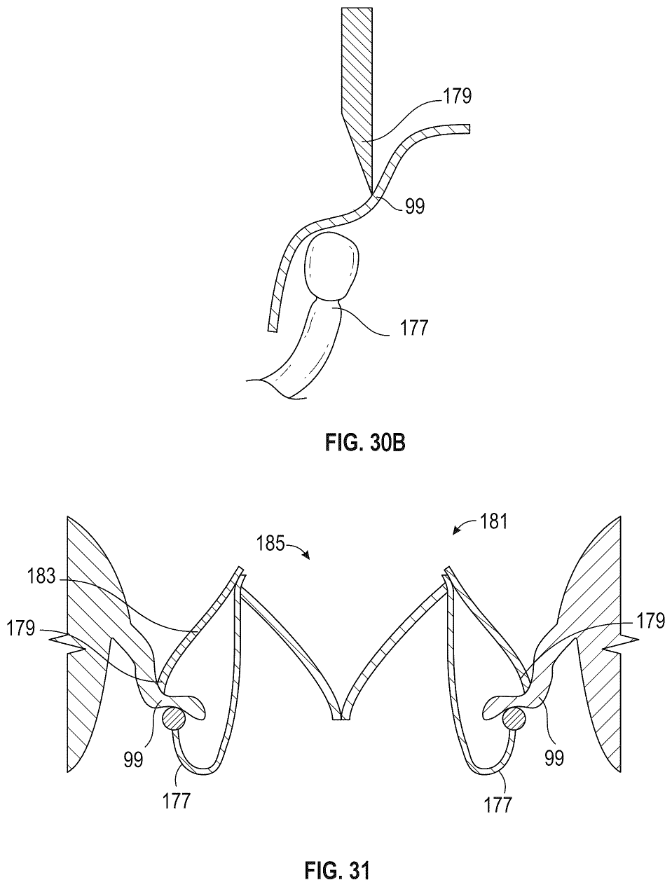

FIGS. 30A and 30B illustrate an example in which the anchor 177 may be positioned radially inward of the grip feature 179. The tip of the anchor 177 may be positioned distal of the grip feature 179. The anchor 177 may press the native valve leaflet 99 towards the grip feature 179 to reduce movement of the native valve leaflet 99 relative to the valve body. The anchor 177 may deflect the native valve leaflet 99 radially inward, as shown in FIG. 30B.

The grip features 179 may be positioned on struts of the frame and may protrude distally as shown in FIG. 30A for example. The grip features 179 may be positioned in a similar manner as the grip features shown in FIGS. 25-28. In examples, the grip features 179 may be positioned in other locations or may have other configurations.

In examples, the use of the grip features may allow for coupling of a prosthetic valve to a native valve. FIG. 31, for example, illustrates an example in which the clamp between the anchor 177 and the grip feature 179 may anchor the prosthetic valve 181 to the native valve. The native valve leaflets 99 may be secured to the prosthetic valve 181.

The use of the grip features may allow an outer diameter of the prosthetic valve 181 to be reduced. The outer diameter of the valve body 183 may be reduced. The outer diameter of the valve body 183, for example, may be less than the diameter of the annulus of the native valve. The outer surface of the valve body 183 accordingly may be spaced from the diameter of the annulus of the native valve. The coupling of the prosthetic valve 181 to the leaflets 99, however, may anchor the prosthetic valve 181 in position relative to the native valve. The leaflets 99 may extend radially inward to the prosthetic valve 181 to reduce fluid flow outside of the flow channel 185 of the prosthetic valve 181.

The use of a valve body 183 having an outer surface that is spaced from the annulus may reduce the possibility of electrical conduction disturbance and other undesired maladies that may result from the prosthetic valve 181. The position of the anchors 177 may further reduce an outward or radial pressure upon the native valve annulus, which may reduce the possibility of a conduction disturbance. The native valve may further be able to reduce in diameter or remodel over time following implantation of the prosthetic valve 181.

In examples, the examples of grip features disclosed herein may be disengaged if desired. For example, a retraction or recapture procedure applied to the prosthetic valve may unclamp one or more of the anchors from the grip features to allow for release of the grip features. The prosthetic valve may be repositioned to a desired location and the grip features may be reengaged as desired.

Other variations in the configurations of the grip features and anchors may be utilized.

The features of FIGS. 19-31 may be utilized solely or in combination with any other example disclosed herein.

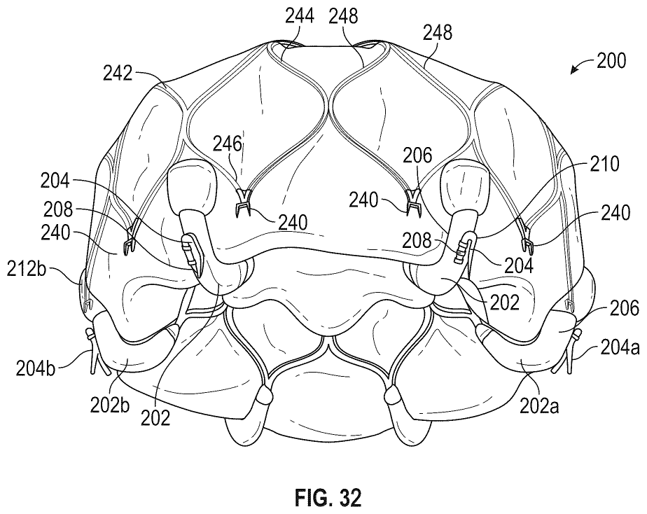

In examples, one or more grip features may be utilized that may be configured to engage a surface of a native valve upon one of the anchors failing to capture one or more native valve leaflets. Referring to FIG. 32, a distal perspective view of a prosthetic valve 200 is shown, including such grip features. The grip features may be provided in a variety of positions as desired.

For example, referring to FIG. 32, one or more of the anchors 202 of the prosthetic valve 200 may include grip features 204. The grip features 204 may be positioned on an arm portion 206 of the respective anchor 202 or on another portion of the anchor 202 as desired. The grip features 204 may be positioned on a portion of an anchor 202 that is configured to face radially outward. Other positions of the grip features 204 may be provided in examples.

The grip features 204 may comprise protrusions or prongs that may extend distally. For example, each grip feature 204 may include a coupling portion 208 for coupling with the anchor 202 and a protrusion 210 that extends distally. The protrusion 210 may have an angled tip or other configuration in examples. The coupling portion 208 may comprise an arm that may be sutured or otherwise coupled to the anchor 202. The protrusion 210 may couple to the coupling portion 208 with a bend or other structure.

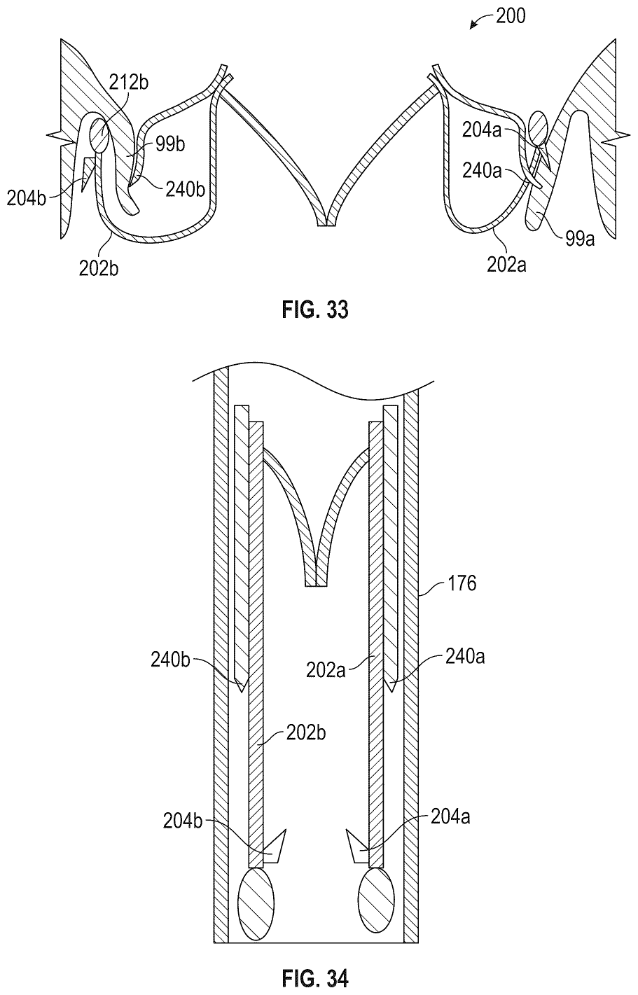

FIG. 33 illustrates an exemplary deployment of the prosthetic valve 200. The anchor 202b has captured the native valve leaflet 99b. As such, the tip 212b of the anchor 202b is positioned radially outward of the native valve leaflet 99b, and the grip feature 204b (configured similarly as the grip features 204) is positioned radially outward of the native valve leaflet 99b that is captured. Accordingly, the grip feature 204b does not engage the leaflet 99b because the grip feature 204b faces radially outward.

Referring to the right side of FIG. 33, the anchor 202a has failed to capture the leaflet 99a. As such, the grip feature 204a (configured similarly as the grip features 204) engages the surface of the native valve leaflet 99a upon the anchor 202a failing to capture the native valve leaflet 99a. The grip feature 204a may penetrate the radially inward facing surface of the native valve leaflet 99a due to the orientation of the grip feature 204a radially outward. The anchor 202a is positioned radially inward of the native valve leaflet 99a. The grip feature 204a may have a greater resistance to a force in a distal direction than in a proximal direction. As such, upon implantation, a force of blood pressure or other force distal upon the prosthetic valve 200 may seat and engage the grip feature 204a in position against the leaflet 99a. The grip feature 204a accordingly may serve to anchor the anchor 202a to the leaflet 99a although the anchor 202a has failed to capture the leaflet 99a.

In examples, the grip features 204 may be configured to face radially inward when the anchors 202 are in an undeployed configuration. FIG. 34, for example illustrates the anchors 202a, b in an undeployed configuration, within a capsule 176 or other retaining member for example. The anchors 202a, b may be elongated or linearized and may extend distally. The grip features 204a, b may face radially inward. A reduced possibility of snagging or snaring against an inner surface of the capsule 176 or other portion of the delivery system may thus result. Upon deployment, the capsule 176 may be retracted to allow the anchors 202 to extend radially outward and invert from the position shown in FIG. 34.

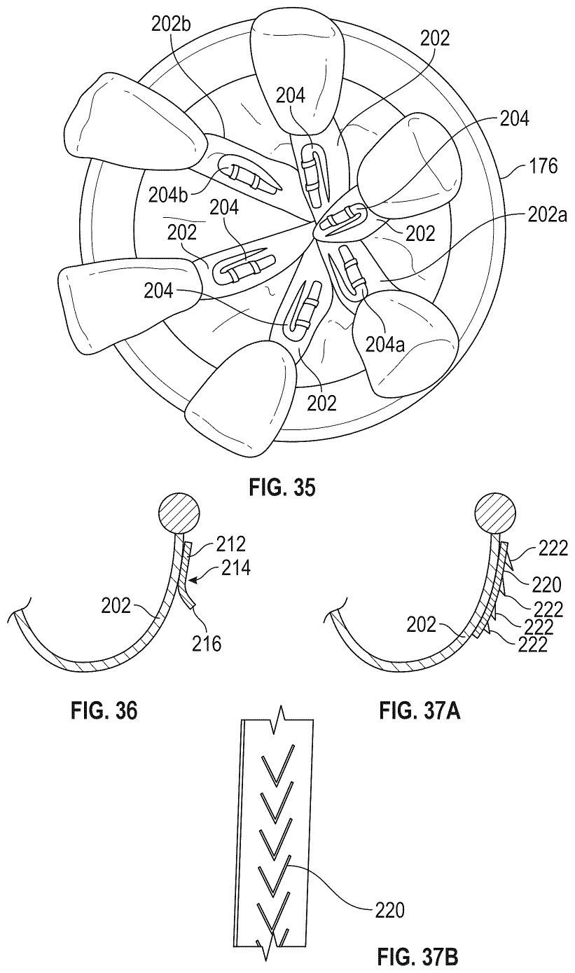

FIG. 35, for example, illustrates the retraction of the capsule 176 to allow the anchors 202 to expand radially outward. The grip features 204 are shown to face radially inward, to reduce the possibility of snagging or snaring against an inner surface of the capsule 176 or other portion of the delivery system. The anchors 202 may bend and invert in orientation upon deployment, such that the grip features 204 face radially outward as desired (as represented in FIG. 33 for example).

The configuration of the grip features 204 may be varied as desired. FIG. 36, for example, illustrates a configuration in which the grip feature 212 comprises a protrusion having a distal end portion 214 bent radially outward to extend to a distal tip 216 positioned radially outward of the anchor 202.

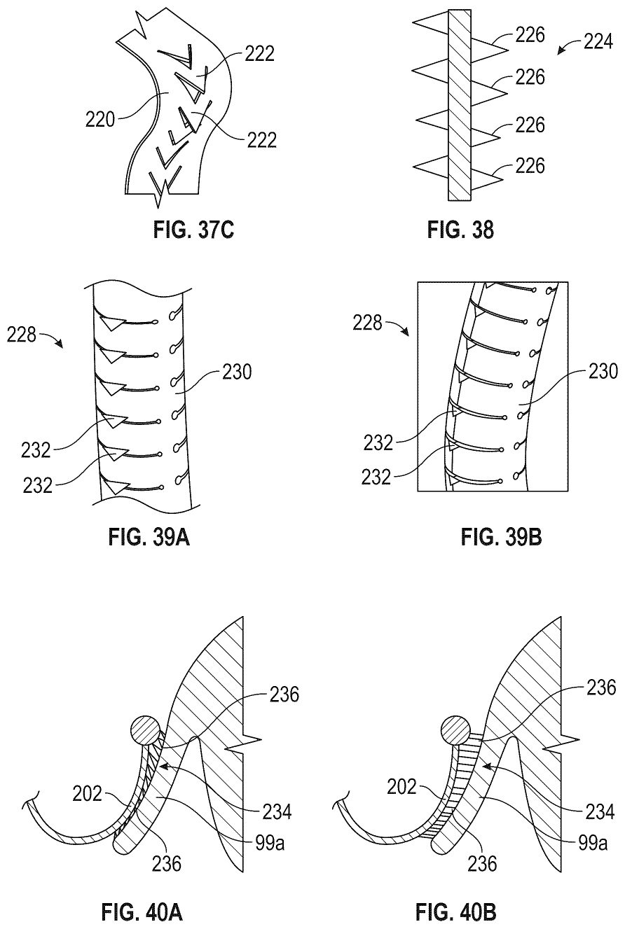

FIGS. 37A-C illustrates a configuration of a grip feature 220 comprising a strip of material having cut-outs to form a plurality of protrusions 222 upon the strip of material being bent. FIG. 37B illustrates the strip of material in a flattened configuration. FIG. 37C illustrates the strip of material upon being bent, with the protrusions 222 extending outward. The material may be bent upon the arm of the anchor 202 as shown in FIG. 37A to cause the protrusions 222 to extend radially outward.

FIG. 38 illustrates a configuration of a grip feature 224 comprising a strip of laterally extending protrusions 226. The strip may be applied to an arm of an anchor 202 for the protrusions 226 to engage a surface of a native valve.

FIGS. 39A and 39B illustrate a configuration of a grip feature 228 comprising a tube 230. The tube 230 may include a plurality of cut outs with edges 232 configured to grip a portion of a native valve upon the tube 230 being deflected. FIG. 39B, for example, illustrates the tube 230 having been deflected with the edges 232 having been exposed. In examples, an outer surface of an anchor may be made of the tube 230, with the edges 232 exposed for engaging a surface of a native valve. The tube 230 may be deflected to expose the edges 232.

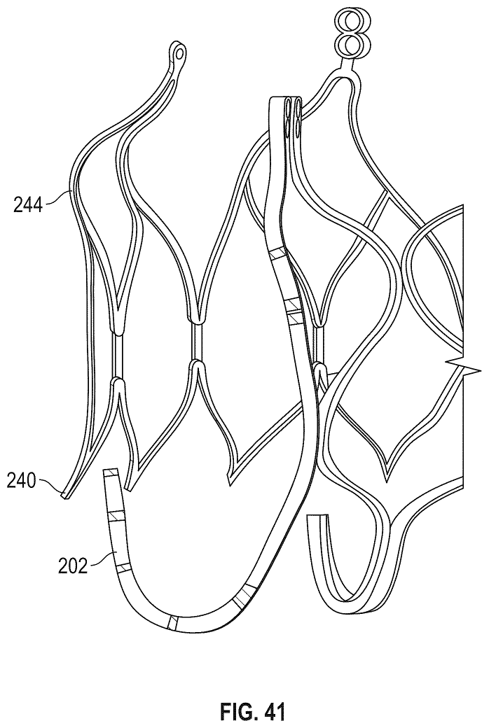

FIGS. 40A and 40B illustrate a configuration of a grip feature 234 comprising one or more filaments 236 or wires. The filaments 236 or wires may protrude radially outward from the anchor 202 and may be configured to be deflectable and smooth when slid proximally along the native valve leaflet 99a and configured to be rigid and engage the native valve leaflet 99a upon a distal sliding movement along the native valve leaflet 99a. FIG. 40B, for example, illustrates the filaments 236 engaging the leaflet 99a upon a distal force being applied to the anchor 202. In such a configuration, the grip feature 234 may slide proximally to be placed in a desired position relative to the leaflet 99a and may then have a distal force applied to the anchor 202 to allow the grip feature 234 to engage the leaflet 99a. The grip feature 234 may be positioned on a tip of an anchor or on a curved or lower loop portion of an anchor as desired.

Other configurations of grip features may be utilized in examples. For example, referring to FIG. 32, one or more grip features 240 may be positioned on the valve body 242. The grip features 240 may comprise prongs that may extend from an outer frame 244 of the valve body 242. The grip features 240 may be positioned in a variety of locations as desired. For example, the grip features 240 may be positioned at the distal apices 246 of the struts 248 of the frame 244. The distal apices 246 may comprise outflow apices of the frame 244. Other positions may be utilized as desired.

In examples, the grip features 240 may be configured to protrude radially outward from the valve body 242 to a greater radial distance than the anchors 202. FIG. 41, for example, illustrates that the outer frame 244 of the valve body 242 or distal apices 246 may protrude radially outward further than the anchors 202. The grip features 240 protrude radially outward further than the anchors 202. As such, upon a failed capture of a leaflet by the anchors, the grip features 240 protrude radially outward to engage the missed leaflet.

FIG. 33, for example, illustrates a utilization of a grip feature 240a (configured similarly as the grip features 240) protruding radially outward to engage the leaflet 99a that the anchor 202a failed to capture. The grip feature 240a may penetrate the inner facing surface of the leaflet 99a to engage the leaflet 99a. Referring to the left side of FIG. 33, the grip feature 240b is positioned to engage the leaflet 99b, although the anchor 202b has captured the leaflet 99b.

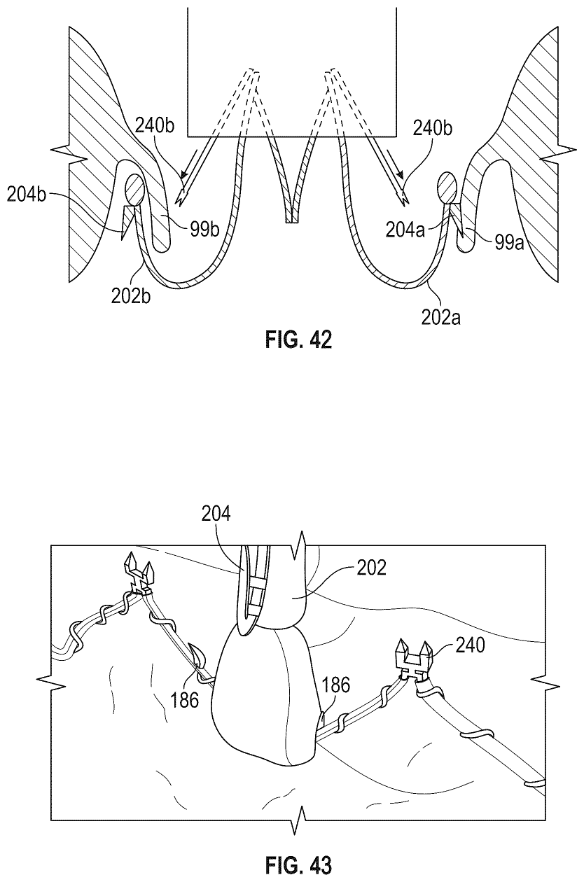

The grip features 240 may deploy in a manner shown in FIG. 42. The anchors 202a, b may be deployed. The anchor 202b has captured the leaflet 99b. The anchor 202a has failed to capture the leaflet 99a. The grip features 240a, b may be deployed to expand radially outward. The grip feature 240a may engage the leaflet 99a to secure and anchor to the leaflet 99a. The grip features 240a, b may be retracted radially inward in a recapture procedure if desired.

The deployment sequence shown in FIG. 42 illustrates use of the grip features 204a, b. However, in examples, the use of the grip features 204a, b may be excluded and the grip features 240a, b may comprise the sole manner to provide anchoring if missed capture of a leaflet occurs. In examples, the grip features 204a, b may comprise the sole manner to provide anchoring if missed capture of a leaflet occurs. The grip features 204 may be utilized solely in combination with the grip features 240, and the grip features 240 may similarly be utilized solely or in combination with the grip features 204.

In an undeployed configuration as shown in FIG. 34, the grip features 204a, b may extend distally, such that the grip features 204a, b do not contact an inner surface of a capsule or other portion of a delivery system.

In examples, combinations of features may be provided. For example, FIG. 43 illustrates use of a grip feature 204 on an anchor 202 in combination with a grip feature 240 on a valve body. Grip features 186 as disclosed in regard to FIGS. 27-32 may further be utilized.

Combinations of features across examples may be utilized.

The features of FIGS. 32-43 may be utilized solely or in combination with any other example disclosed herein.

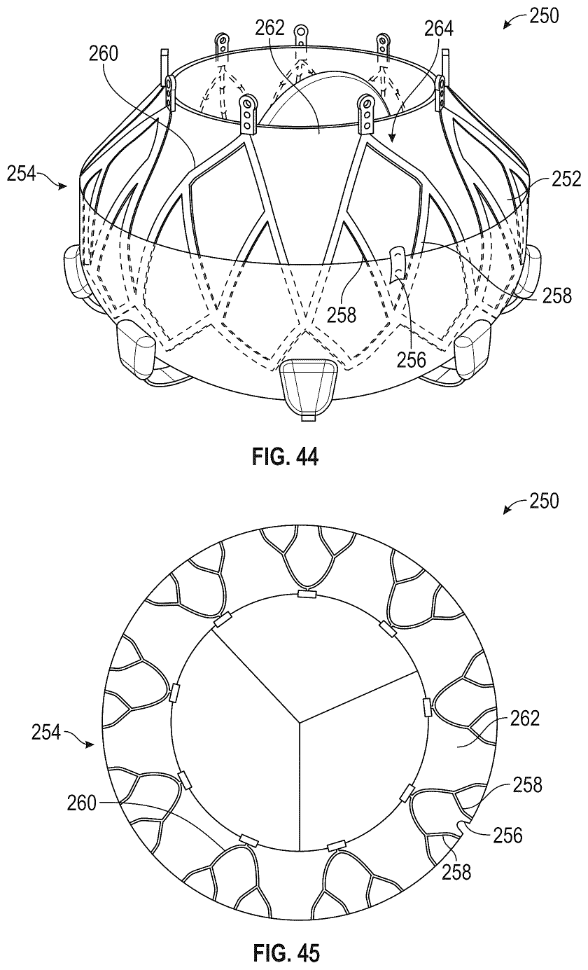

In examples, a prosthetic valve may include a valve body having an outer surface, and the outer surface of the valve body may include a channel for a pacemaker lead to be passed through. For example, referring to FIG. 44, a perspective view of a prosthetic valve 250 is shown. The prosthetic valve 250 may include features similar to other prosthetic valves disclosed herein, unless otherwise stated.

The prosthetic valve 250 may include a valve body 252 having an outer surface 254. The outer surface 254 of the valve body 252 may comprise the surface configured to contact the native valve. The outer surface 254, in examples, may comprise a sealing surface configured to form a seal with the native valve. For example, the outer surface 254 may comprise an outer surface of a sealing body configured to form a seal with a native valve.

In examples, the outer surface 254 may include the channel 256 for the pacemaker lead to be passed through. The channel 256 may be configured for the pacemaker lead to be passed through in a longitudinal manner, from a proximal portion of the prosthetic valve 250 towards a distal portion of the prosthetic valve 250.

The channel 256 may be positioned between struts 258 of the frame 260 of the valve body 252. The struts 258, for example, may be separated by openings, similar to other examples of frames disclosed herein. The channel 256 may extend through one of the openings in examples. The channel 256 may comprise a recess in a skirt 262 that may be positioned upon the frame 260. The skirt 262, for example, may comprise a sealing skirt configured to form a seal with the native valve. The position of the channel 256 between the struts 258 of the frame 260 may allow the skirt 262 to indent at the channel 256 due to the lack of presence of a strut 258. The channel 256 may comprise a recess in the outer surface 254 of the valve body 252 and the skirt 262. The skirt 262 may be configured to deflect inward to form the channel 256. The deflection may occur upon a pacemaker lead being passed between the outer surface 254 of the valve body 252 and an inner surface of a native valve (such as the valve annulus).

In examples, a proximal surface 264 of the skirt 262 may comprise a tapered surface. The tapering of the proximal surface 264 may allow a pacemaker lead to more easily extend towards the channel 256 upon insertion distally towards the prosthetic valve 250. The taper of the surface may comprise a guide feature for guiding the pacemaker lead to the channel 256.

FIG. 45 illustrates a top view of the prosthetic valve 250 showing the indentation of the channel 256 for example.

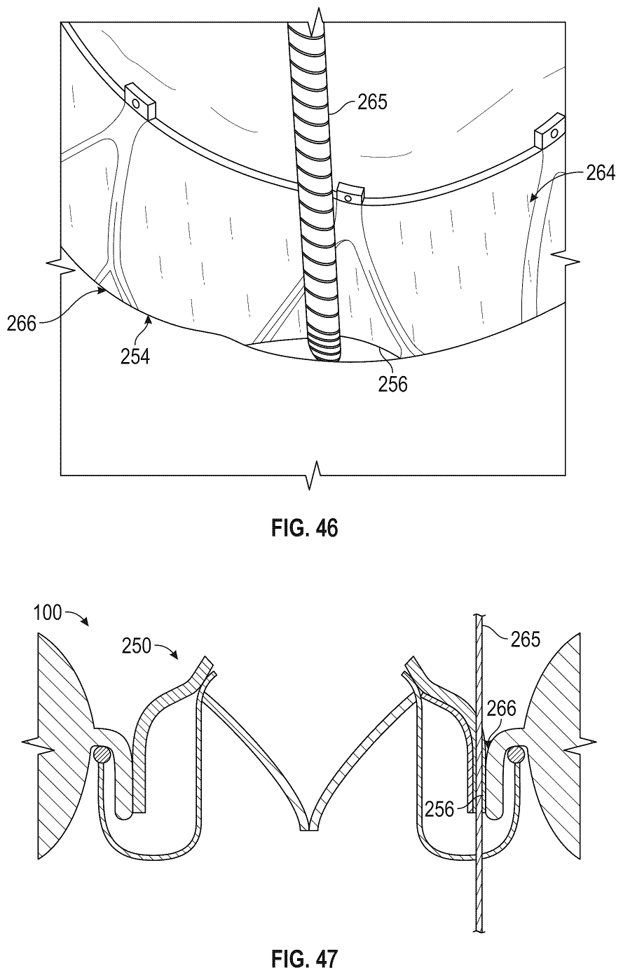

The prosthetic valve 250 may be implanted to an implantation site. Upon implantation, the outer surface 254 of the valve body 252 may be in contact with the native valve. A pacemaker lead 265 may be inserted adjacent to the prosthetic valve 250 either during the implantation procedure or during a subsequent procedure. FIG. 46, for example, illustrates the prosthetic valve 250 implanted, with the outer surface 254 of the valve body 252 positioned against the native valve. The channel 256 may be positioned to allow a pacemaker lead 265 to be passed through, to extend to the distal side or ventricular side of the prosthetic valve 250. The channel 256 is positioned between the outer surface 254 of the valve body 252 and an inner surface 266 of the native valve. The outer surface 254 of the valve body 252 and the inner surface 266 of the native valve may form a seal about the pacemaker lead extending through the channel 256.

FIG. 47 illustrates a side cross sectional schematic view of the prosthetic valve 250 implanted to a native valve 100. The pacemaker lead 265 is shown passing through the channel 256 and between the outer surface 254 of the valve body 252 and the inner surface 266 of the native valve. The pacemaker lead 265 may beneficially not pass through the central flow channel of the prosthetic valve, as such positioning may impede the operation of the prosthetic valve leaflets.

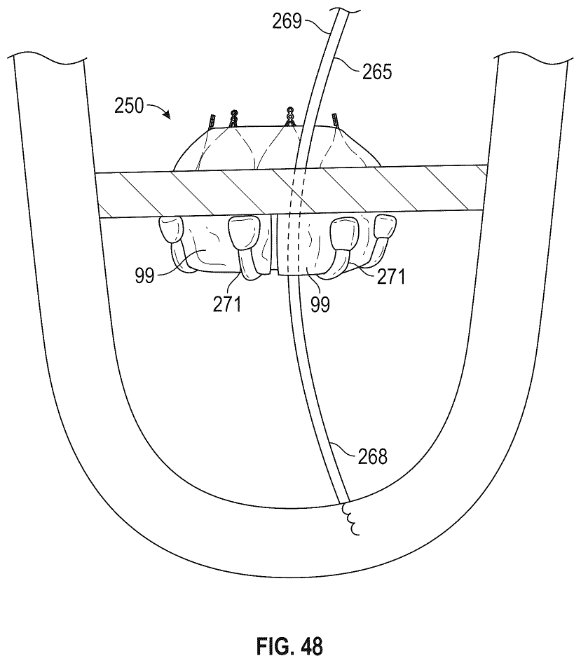

FIG. 48 illustrates a side schematic view of the pacemaker lead 265 passed through the channel 256 to the distal or ventricular side of the prosthetic valve 250. A distal end portion 268 of the pacemaker lead 265 may be implanted at a desired location within a ventricle. A proximal end portion 269 of the pacemaker lead 265 may couple to a pacemaker. The pacemaker lead 265 may be positioned within the channel 256, which may be circumferentially positioned between two of the plurality of anchors 271. The position between the adjacent anchors 271 may aid to secure the pacemaker lead 265 in position adjacent to the prosthetic valve 250. The position between the adjacent anchors 271 may seal the pacemaker lead 265 extending through the channel 256.

In examples, one or more of the grip features of FIGS. 19-31 may be utilized with the prosthetic valve 250. The grip features may aid to secure the prosthetic valve 250 in position upon the pacemaker lead being advanced distally through the channel 256.

The features of FIGS. 44-48 may be utilized solely or in combination with any other example disclosed herein.

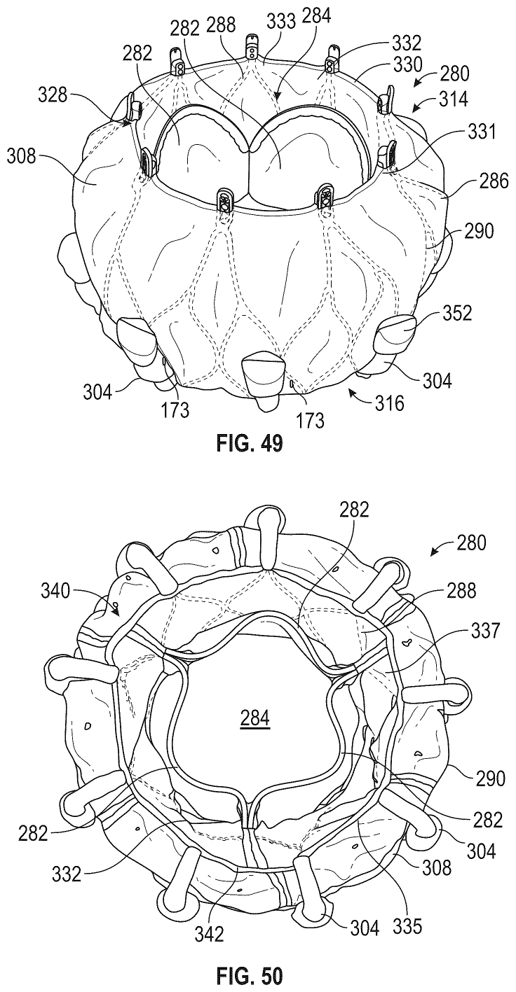

FIG. 49 illustrates a perspective view of a prosthetic valve 280 that may be utilized in any example herein. The prosthetic valve 280 may include the features discussed in regard to prosthetic valve 10, prosthetic valve 112, or prosthetic valve 250, unless stated otherwise. The prosthetic valve 280 may include the features of any other prosthetic valve or example disclosed herein.

The prosthetic valve 280 includes one or more prosthetic valve leaflets 282 (marked in FIG. 50) that may be configured similarly as the prosthetic valve leaflets 16 disclosed in regard to FIGS. 1-7G. The prosthetic valve leaflets 282 may be positioned within a flow channel 284 (marked in FIG. 50) of the prosthetic valve 280. The prosthetic valve leaflets 282 are supported by a valve body 286 and may extend radially inward from the valve body 286 in the flow channel 284.

The valve body 286 may include an inner body 288 (marked in FIG. 50) and an outer body 290. The inner body 288 may include the features of other examples of inner bodies disclosed herein unless stated otherwise. The inner body 288 may include an inner frame 292 (marked in FIGS. 51, 53, and 54). The inner frame 292 supports the prosthetic valve leaflets 282. Referring to FIG. 51, the inner frame 292 may include a plurality of struts 294 that may be separated by spaces or openings 296, in a similar manner as other examples of inner frames disclosed herein. The inner frame 292 may include a proximal end portion 298 and a distal end portion 300. The inner frame 292 may include one or more couplers 299 for coupling with the outer body 290. The one or more couplers 299 may be positioned at the proximal end 301 of the inner frame 292 or another location as desired.

In examples, the inner frame 292 may have a wider proximal end portion 298 than a central portion 302 of the inner frame 292, and a wider distal end portion 300 than the central portion 302 of the inner frame 292. The inner frame 292 may curve outward from the central portion 302 to the wider proximal end portion 298, and may curve outward from the central portion 302 to the wider distal end portion 300. The inner frame 292 may have a substantially hourglass shape or profile, which may be similar to the shape or profile of the inner frame 116 shown in FIG. 10 for example. The distal end portion 300 of the inner frame 292 may couple to the anchors 304.

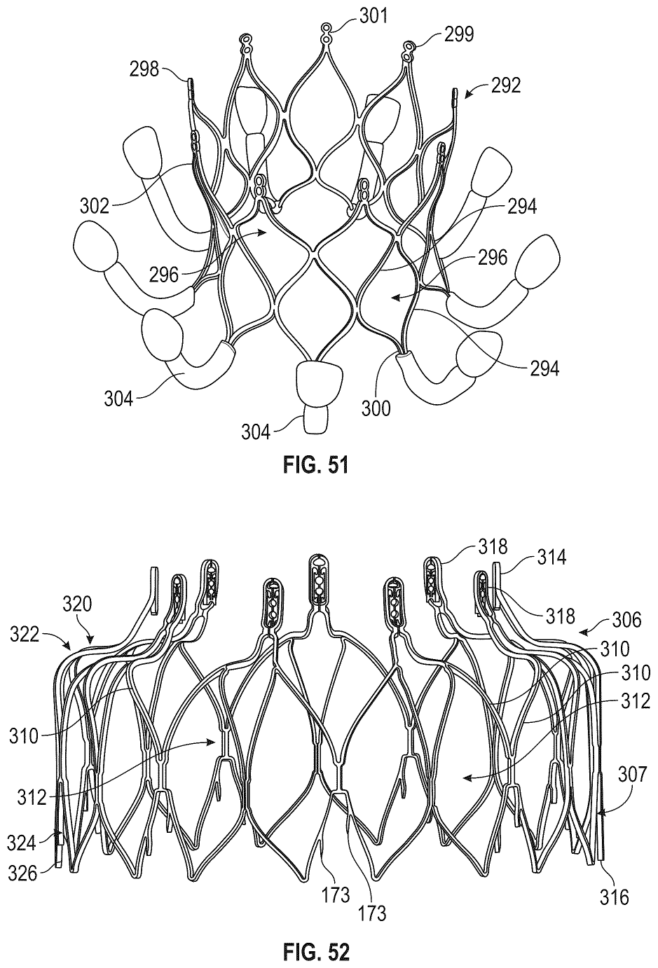

The outer body 290 may include the features of other examples of outer bodies disclosed herein unless stated otherwise. The outer body 290 may comprise a sealing body, and may include an outer frame 306 (marked in FIG. 52) and a sealing skirt 308, outer skirt, or fabric skirt positioned upon the outer frame 306. The outer frame 306 may be positioned radially outward of the inner frame 292. An outer surface 307 of the outer frame 306 faces radially outward from the prosthetic valve 280. The outer surface 307 is for pressing against tissue of the native heart valve. The sealing skirt 308 extends along the outer surface 307 of the outer frame 306.

FIG. 52 illustrates the outer frame 306 in isolation from other features of the prosthetic valve 280. The outer frame 306 may include a plurality of struts 310 that may be separated by spaces or opening 312, in a similar manner as other examples of outer frames disclosed herein. The struts 310 may form the openings 312 between the struts 310. The struts 310 may form expandable and collapsible cells. The outer frame 306 may include a proximal end portion 314 and a distal end portion 316. The proximal end portion 314 may include a coupling portion having one or more couplers 318 adapted for coupling with the inner frame 292, and particularly with the couplers 299 of the inner frame 292. The couplers 318 may include eyelets in examples that may be disposed along a proximal, inflow end portion, or upstream portion of the frame 306 for receiving sutures. The prosthetic heart valve may be deployed by being coupled to the sutures (e.g., a tether assembly), with tension released in the sutures to deploy the valve. The prosthetic heart valve may be retrieved after deployment by applying tension to the sutures.

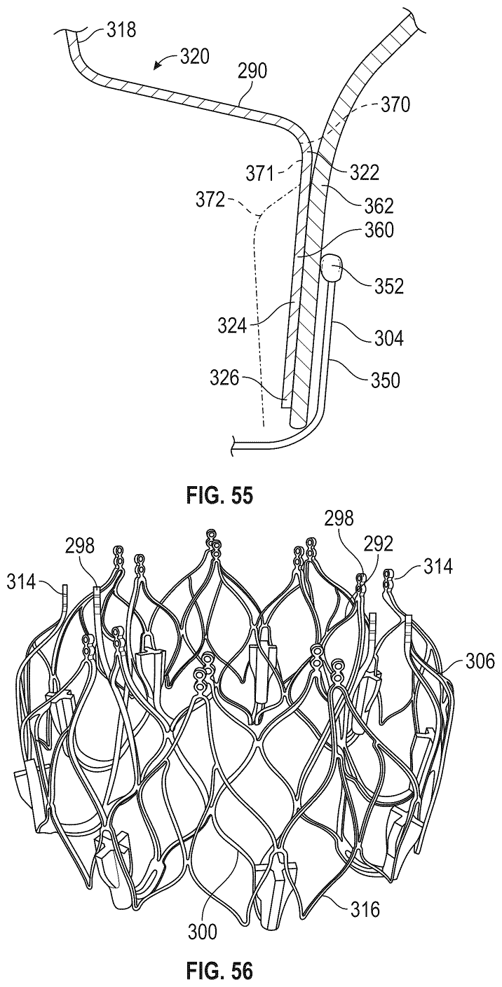

The proximal end portion 314 of the outer frame 306 may be coupled to the proximal end portion 298 of the inner frame 292. The outer frame 306 may protrude radially outward from the coupling portion to a first tapered portion 320. The first tapered portion 320 may extend radially outward from the coupling portion and taper at a first angle (with the angle more clearly represented in FIGS. 54 and 55). The first tapered portion 320 may extend radially outward to a curve portion or bend portion or intermediate portion 322.

The curve portion or bend portion or intermediate portion 322 may comprise a curve or bend that angles the first tapered portion 320 relative to a second tapered portion 324. The intermediate portion 322 may angle the first tapered portion 320 relative to the second tapered portion 324 such that the second tapered portion 324 extends axially. The angle of the intermediate portion 322, for example, may be seventy degrees or may be a greater angle (e.g., eighty degrees, ninety degrees, 100 degrees, 110 degrees), to vary the angle between the first tapered portion 320 and the second tapered portion 324. The second tapered portion 324 may taper at a second angle (with the angle more clearly represented in FIGS. 54 and 55) that is different than the first angle (the angle of the first tapered portion 320). In examples, the second tapered portion 324 may taper radially inward (as shown in FIG. 55). As shown in FIGS. 54 and 55, the second tapered portion 324 may be angled to extend radially inward in a direction from the intermediate portion 322 to the distal end 326 of the outer frame 306. The angle of the second tapered portion 324 may be a linear angle (e.g., a straight angle as represented in FIG. 55 for example) or the second tapered portion 324 may have a curvature in examples. Other configurations of outer frames (e.g., other forms of outer frames disclosed herein) may be utilized as desired.

In examples, the outer frame 306 has a tapered shape such that a downstream portion or distal end portion 316 has a smaller diameter than the intermediate portion 322 of the outer frame 306. The intermediate portion 322, for example, may have a diameter in a range of about 35 millimeters to about 60 millimeters, with the distal end portion 316 having a lesser diameter.

The outer frame 306 may include one or more grip features 173 as disclosed herein. The grip features 173 may be configured similarly as the grip features 173 shown in FIG. 29A or may be configured similarly as any other form of grip feature disclosed in regard to FIGS. 19-31. The grip features 173 may operate in a similar manner as discussed in regard to any of the examples of FIGS. 19-31 as desired. The grip features may comprise barbs in examples (or may have other forms as disclosed herein). The barbs may be disposed along the struts of the outer frame 306. The barbs may extend through the sealing skirt 308 for penetrating the tissue of the native heart valve. In examples, the prosthetic valve 280 may include any of the grip features of FIGS. 32-43 as desired. Any other feature of any other example disclosed herein may be utilized as desired.

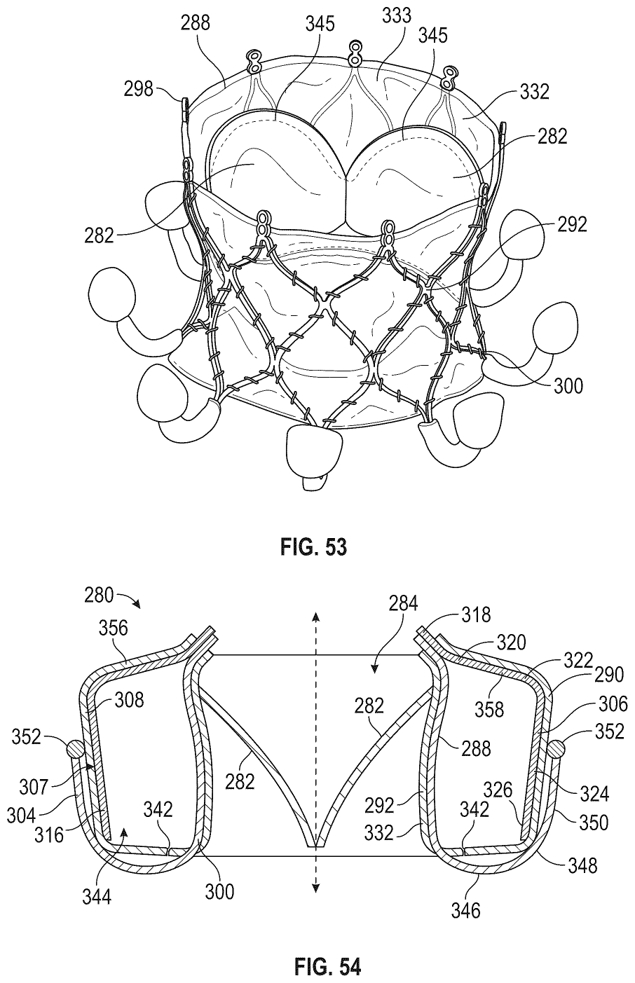

Referring to FIG. 49, the sealing skirt 308 or outer skirt may extend over the outer surface 307 of the outer frame 306 from the proximal end portion 314 of the outer frame 306 to the distal end portion 316 of the outer frame 306. The sealing skirt 308 may comprise a continuous or unitary piece of material or fabric that extends from the proximal end portion 314 to the distal end portion 316 along the outer surface 307 of the outer frame 306. A suture line or stitch line 330 is shown at the proximal end portion 328 of the prosthetic valve 280 coupling a proximal end 331 of the sealing skirt 308 with a proximal end 333 (marked in FIG. 53) of an inner skirt 332.

Referring to FIG. 50, the distal end 335 of the sealing skirt 308 or outer skirt may couple to a distal end 337 of the inner skirt 332 at a distal end portion 340 of the prosthetic valve 280. A suture line or stitch line 342 may be positioned at the distal end portion 340 of the prosthetic valve 280. The sealing skirt 308 or outer skirt may extend across or span the gap 344 between the distal end portion 300 of the inner frame 292 and the distal end portion 316 of the outer frame 306. The suture line or stitch line 342 may be positioned at a location that may improve ease of assembly for the prosthetic valve 280, as the suture line or stitch line 342 may be more accessible at the distal end portion 340 of the prosthetic valve 280 than at an interior portion (e.g., an interior surface facing the flow channel 284).

FIG. 53 illustrates the configuration of the inner skirt 332. The inner skirt 332 may extend from the proximal end portion 298 of the inner frame 292 to the distal end portion 300 of the inner frame 292. The prosthetic valve leaflets 282 may be sutured or otherwise coupled to the inner skirt 332. Suture lines or stitch lines 345 between the prosthetic valve leaflets 282 and the inner skirt 332 are shown. The inner skirt 332 may extend along the interior surface or radially inward facing surface of the inner frame 292. The inner skirt 332 may include any of the features of the skirts disclosed in regard to FIGS. 1-7G, including materials for reducing or promoting tissue formation or thrombus formation, or any of the constructions of the skirts disclosed in regard to FIGS. 1-7G. The outer skirt or sealing skirt 308 may include any of the features of the skirts disclosed in regard to FIGS. 1-7G, including materials for reducing or promoting tissue formation or thrombus formation, or any of the constructions of the skirts disclosed in regard to FIGS. 1-7G. Other forms of skirts may be utilized in examples.

The anchors 304 may include the features of any other form of anchor disclosed herein unless stated otherwise. The anchors 304, for example, may have hook shapes or may be hook arm anchors as disclosed in regard to FIGS. 1-11. The anchors 304 may include the features of the anchors 44, 126 unless stated otherwise. The anchors 304 may include the features of any other anchors (e.g., the anchors disclosed in regard to FIGS. 12-48 unless stated otherwise). The anchors 304 may be coupled to the distal end portion 300 of the inner frame 292. The anchors 304 may extend radially outward from the distal end portion 300 of the inner frame 292 and may extend across or span the gap 344 between the distal end portion 300 of the inner frame 292 and the distal end portion 316 of the outer frame 306. The anchors 304 are adapted to hook over a native valve leaflet to anchor to the native valve. The distal end portion 300 of the inner frame 292 is spaced from the distal end portion 316 of the outer frame 306 with the gap 344.

Referring to FIG. 54, the anchors 304 may each include a coupling portion 346, a drop loop 348 positioned radially outward of the coupling portion 346, and a tip portion 350 including a tip 352 of the respective anchor 304. The tip portion 350 may extend in an axially proximal direction or inflow direction of the prosthetic valve 280 from the drop loop 348. The drop loop 348 may comprise a curved portion or bend portion that may angle the coupling portion 346 relative to the tip portion 350. The anchors 304 each protrude radially outward from the outer surface of the valve body 286. In examples, the tips 352 of the respective anchors 304 may be positioned radially outward of the outer surface 307 of the outer frame 306 (and the outer surface of the sealing skirt 308). The tips 352 of the respective anchors 304 may overlap the outer surface 307 of the outer frame 306 (and the outer surface of the sealing skirt 308). Referring to FIG. 49, the tips 352 of the anchors 304 may be positioned between the “V” shaped openings of the struts of the outer frame 306.

FIG. 54 illustrates a cross sectional view of the prosthetic valve 280 showing the skirt 356 of the prosthetic valve 280 (the combination of the sealing skirt 308 and the inner skirt 332) positioned upon the frame 358 of the prosthetic valve 280 (the combination of the inner frame 292 and the outer frame 306). In examples, the skirt 356 may be tightly coupled to the frame 358 of the prosthetic valve 280 such that the skirt 356 tensions the frame 358. The tension applied by the skirt 356 to the frame 358 may produce or vary the taper of the outer frame 306. Tension may be applied by the anchors 304 in examples in combination with or alternatively from the skirt 356.

The resulting taper of the outer frame 306 may be a taper as shown in FIGS. 54 and 55, due to the tension provided by the skirt 356. Such a configuration may have a variety of benefits. Referring to FIG. 55, for example, the taper of the second tapered portion 324 may be at a linear angle (e.g., a straight angle) radially inward. A large linear and continuous contact area 360 (forming a conical frustum shape for the contact area of the prosthetic valve 280 around the circumference of the valve 280) against the native valve leaflet 362 or other portion of the native valve (e.g., the annulus) may be provided. Improved sealing against the native valve leaflet 362 or other portion of the native valve may result. Annular engagement is improved. Further, the tip portion 350 of the anchor 304 may be linear and may match or have the same angle as the angle of the second tapered portion 324. As such, improved contact between the tip portion 350 and the outer surface of the outer body 290 may result. The native valve leaflet 362 may be compressed or clamped between the tip portion 350 and the outer surface of the outer body 290. The compression may be between the outer surface 307 of the outer frame 306 or the outer surface of the sealing skirt 308 or outer skirt in examples. The tip portion 350 and/or outer body 290 may be biased to contact or press against each other to provide compression or clamping of the native valve leaflet 362. Compression or clamping configurations as disclosed in regard to FIGS. 19-31 may be utilized.

A configuration as shown in FIG. 55 may differ from a configuration including a protruding shoulder 370 forming a rib protruding convex or parabolic radially outward (with a such a profile indicated in dashed lines in FIG. 55), with a double or “S” curvature (with a first curve marked with 371 and a second opposite curve marked with 372). As such, a protruding shoulder forming a rib extending circumferentially about the prosthetic valve may be excluded. A single direction bend for the curve portion or bend portion 322 may be utilized.

The features of the prosthetic valve of FIGS. 49-55 may be utilized solely or in combination with any other example disclosed herein.

Variations in the configuration of the prosthetic valve 280 may be provided. FIG. 56, for example, illustrates a variation in which the outer frame 306 is not directly coupled to the inner frame 292. The proximal end portion 314 of the outer frame 306 is not directly coupled to the proximal end portion 298 of the inner frame 292. Further, the distal end portion 316 of the outer frame 306 is not directly coupled to the distal end portion 300 of the inner frame 292.

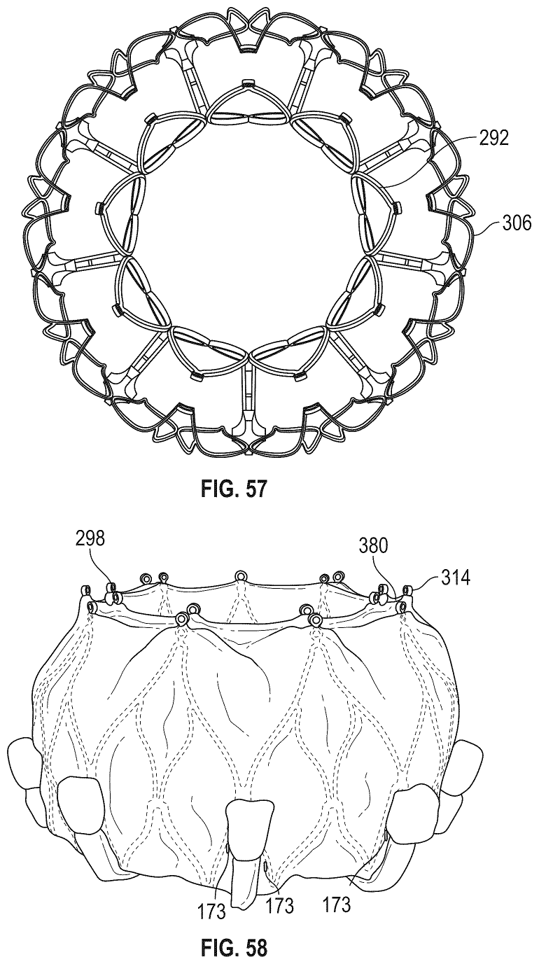

FIG. 57 illustrates a top view of the separation or lack of direct contact or coupling between the outer frame 306 and the inner frame 292.

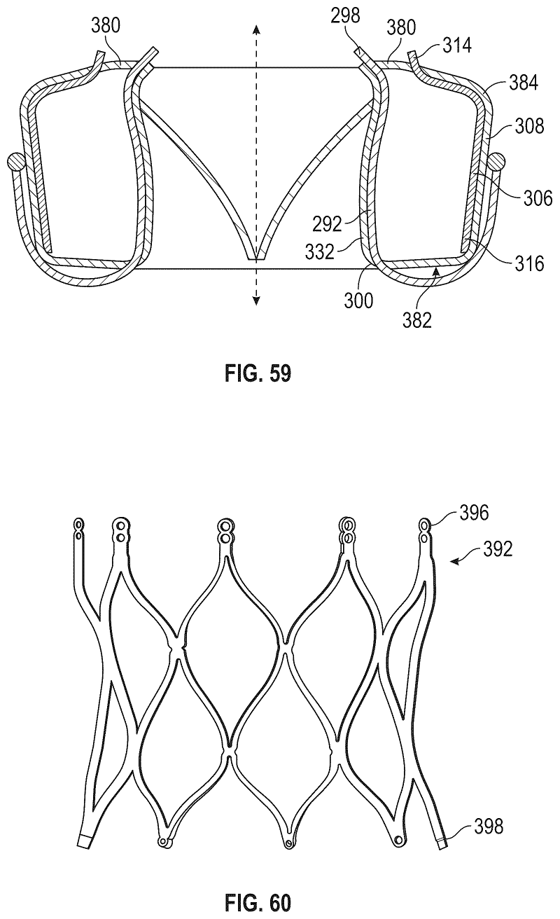

Referring to FIGS. 58 and 59, a connection between the frames 292, 306 may be provided by a flexible body, connecting member, connecting skirt or intermediate component 380 that may extend between the proximal end portion 298 of the inner frame 292 and the proximal end portion 314 of the outer frame 306. The intermediate component 380 may allow movement of the inner frame 292 to the outer frame 306. The connecting skirt or intermediate component 380 may comprise a continuation of the sealing skirt 308 or the inner skirt 332 and may extend radially between the proximal end portion 298 of the inner frame 292 and the proximal end portion 314 of the outer frame 306. The outer frame 306 accordingly may be coupled to the inner frame 292 with the connecting skirt or intermediate component 380 and with the distal end portion 382 of the skirt 384 of the prosthetic valve. The connecting skirt or intermediate component 380 may comprise a proximal end portion of the skirt 384 and the distal end portion 382 of the skirt 384 may comprise a connecting skirt for the distal end portions 300, 316 of the frames 292, 306. The connecting skirt or intermediate component 380 and the distal end portion 382 of the skirt 384 may comprise flexible tether connections between the proximal end portion 298 of the inner frame 292 and the proximal end portion 314 of the outer frame 306.

The flexible connections provided by the connecting skirt or intermediate component 380 and the distal end portion 382 of the skirt 384 may allow the outer frame 306 to move axially relative to the inner frame 292 and to tilt laterally relative to the inner frame 292. Improved compliance to the shape of a native valve and conformability may result. Improved sealing with the native valve accordingly may result. Reduced radial stiffness may further result.

The features of the prosthetic valve of FIGS. 56-59 may be utilized solely or in combination with any other example disclosed herein.

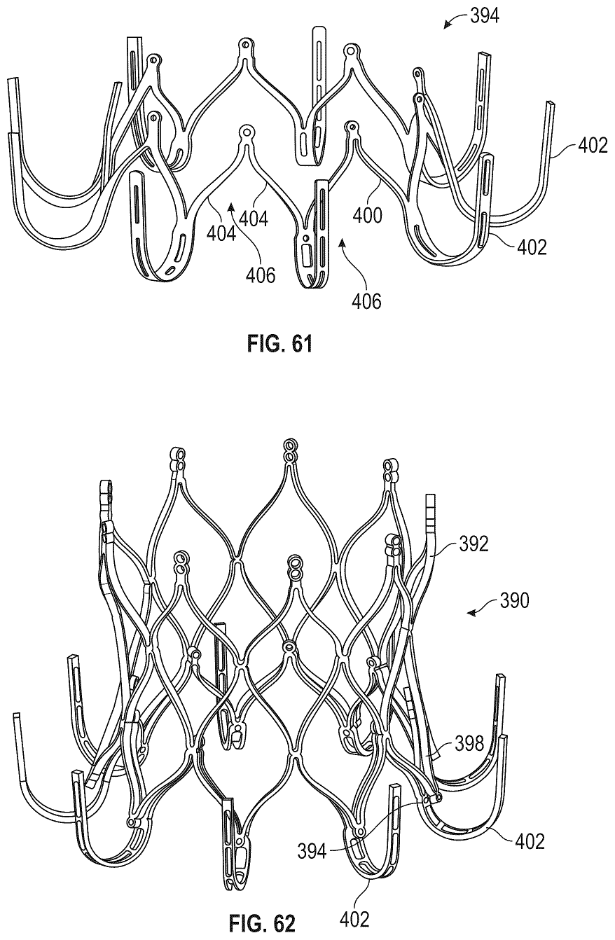

Additional variations in the configuration of the prosthetic valve 280 may be provided. FIGS. 60-63, for example, illustrate a variation in which the inner frame 390 (shown in perspective view in FIG. 62) includes two portions 392, 394 (or a first frame and a second frame) that are formed non-integral with each other. FIG. 60 illustrates a side view of a first portion 392 comprising a sleeve portion or cylindrical portion of the inner frame 390, configured similarly as the inner frame 292 shown in FIG. 51. The first portion 392 may include struts and surround a flow channel for the prosthetic valve leaflets 282. The first portion 392 extends axially from a proximal end portion 396 to a distal end portion 398. The first portion 392 comprises a sleeve portion that supports the prosthetic valve leaflets 282, and features of the proximal end portion 396 and distal end portion 398 correspond to the respective features of the proximal end portion 298 and distal end portion 300 of the inner frame 292.

A perspective view of the second portion 394 or second frame is illustrated in FIG. 61. The second portion 394 may include a supporting frame 400 that is coupled to anchors 402, which may be configured similarly as the anchors 304 unless stated otherwise. The supporting frame 400 may comprise a ring for surrounding the flow channel for the prosthetic valve leaflets 282. The supporting frame 400 may include a plurality of longitudinally extending struts 404, with openings 406 between the struts 404 and bounded by the struts 404. The second portion 394 may include the struts of the ventricular anchors.

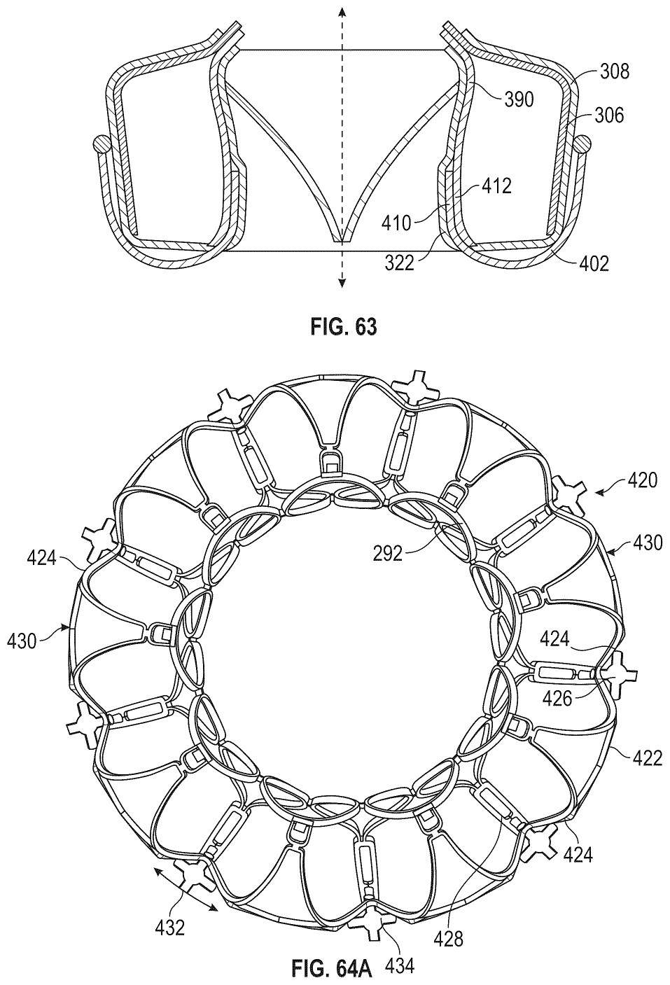

The second portion 394 may be made of a material having a lesser thickness 410 (marked in FIG. 63) in the radial dimension than the thickness 412 (marked in FIG. 63) of the material forming the first portion 392. A lesser radial thickness is utilized. As such, each of the components shown in FIG. 61 may have a lesser thickness 410 (in the radial dimension) than the thickness 412 of the components shown in FIG. 60. Such a feature may beneficially reduce the stiffness of the arms forming the anchors 402 in FIG. 61, to increase compliance to an implantation site. The anchors 402 may be more deflectable inward and outward, and reduced radial stiffness may result. The first portion 392 illustrated in FIG. 60 may have a greater stiffness to support the prosthetic valve leaflets 282 with the flow channel.

The first portion 392 may be coupled to the second portion 394 as shown in FIG. 62 for example. Sutures connections or other forms of coupling may couple the first portion 392 to the second portion 394. The supporting frame 400 may be positioned at the distal end portion 398 of the inner frame 390 and may be positioned radially inward of the inner frame 390 (or may be positioned radially outward in examples). The anchors 402 may protrude radially outward.

FIG. 63 illustrates a cross sectional view of a prosthetic valve utilizing the inner frame 390.

The features of the prosthetic valve of FIGS. 61-63 may be utilized solely or in combination with any other example disclosed herein.

Additional variations in the configuration of the prosthetic valve 280 may be provided. In examples, the prosthetic valve may be configured such that the anchors utilized have a same or lesser radius than an outer surface of the outer body. In examples, the anchors may be tucked or nested within the outer body.

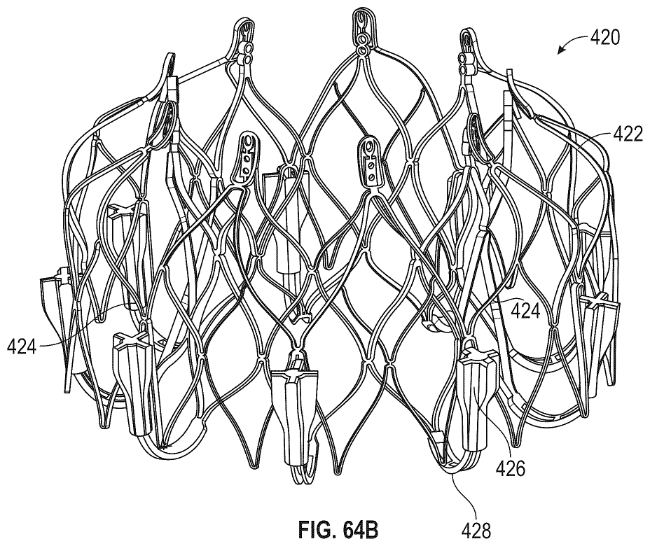

For example, referring to FIG. 64A, a top view of a configuration of a frame 420 is illustrated, including an outer frame 422 having indentations, pockets, or recesses 424 for receiving the tips 426 of the anchors 428. The anchors 428 may be configured similarly as the anchors 304 unless stated otherwise.

The recesses 424 may comprise shaped arcuate sections of the outer frame 422 protruding radially inward from the outermost surface 430 of the outer frame 422. The recesses 424 may be spaced circumferentially from each other about the outer surface of the outer frame 422 and positioned to circumferentially align with the positions of the anchors 428. The recesses 424 may be elongate and may extend axially along the outer frame 422. Axially extending concave recesses 424 for receiving the anchors 304 may be utilized. The circumferential width 432 of each of the recesses 424 may be set to be at or greater than the width of each of the tips 426. The radial depth 434 of each of the recesses 424 may be set to be at or greater than the radial thickness of each of the tips 426 of the anchors 428. Each of the recesses 424 may be configured to receive the tips 426 of the anchors 428, with the tip 426 being at a same or lesser radius than an outermost surface 430 of the outer frame 422. In examples, the recesses 424 may be configured such that the tips 426 of the anchors 428 are partially or at least partially recessed inward of the outer frame 422.

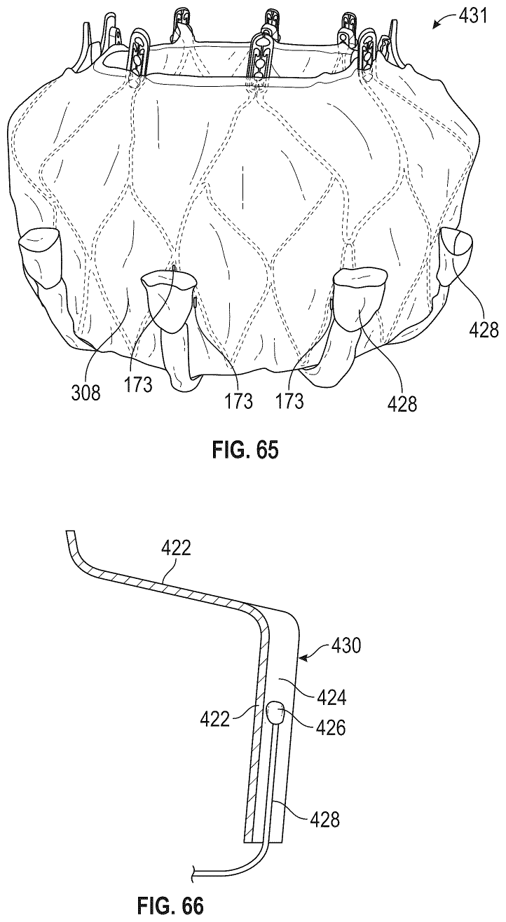

FIG. 64B illustrates a side perspective view of the configuration shown in FIG. 64A. FIG. 65 illustrates a perspective view of a prosthetic valve 431 utilizing the frame 420, with the sealing skirt 308 positioned upon the outer surface of the outer frame 422. FIG. 66 illustrates a side cross sectional schematic view of tip 426 within a recess 424. The tip 426 is shown to protrude to a radius that is the same or less than the radius of the outermost surface 430 of the outer frame 422. The sealing skirt 308 may be positioned between the tip 426 and the outer frame 422.

The configuration as shown in FIGS. 64A-66 may beneficially reduce the outer profile or outer diameter of the prosthetic valve. A reduced force directed radially outward may result. One or more leaflets engaged by the anchors 428 may be pressed into a respective recess 424 and secured within the recess 424. In examples, grip features as disclosed herein (e.g., as disclosed in FIGS. 19-31 and 52, or other forms of grip features) may be utilized to secure the leaflet in the space between the tip 426 of the anchor 428 and the outer frame 422.

The features of the prosthetic valve of FIGS. 64A-66 may be utilized solely or in combination with any other example disclosed herein.

Alternative configurations may be provided such that the anchors utilized have a same or lesser radius than an outer surface of the outer body, or that the anchors are tucked or nested within the outer body.

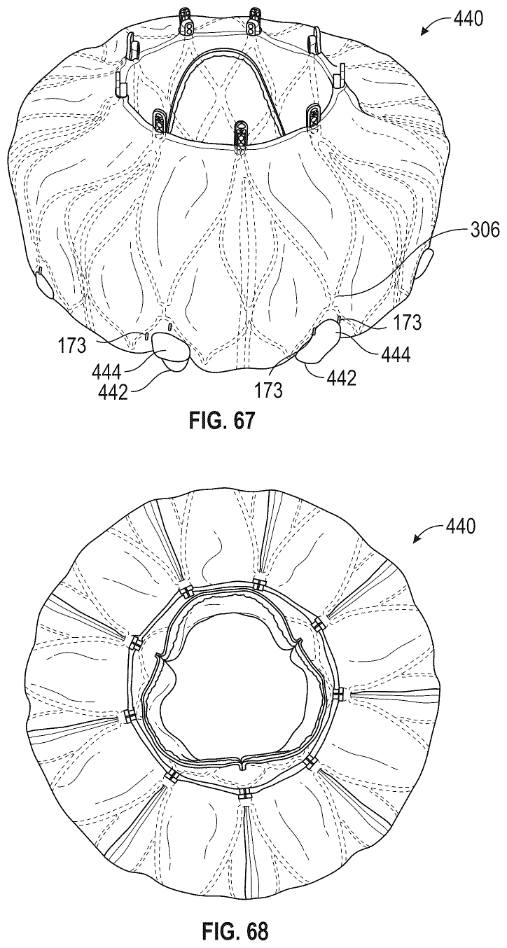

Referring to FIG. 67, a perspective view of a prosthetic valve 440 is illustrated. The prosthetic valve 440 may include the features of the prosthetic valve 280 unless stated otherwise. The prosthetic valve 440 has anchors 442 that each have a tip 444 positioned distal of the outer frame 306 (shown in FIG. 70). The position of the tip 444 distal (or in the outflow direction) of the outer frame 306 allows the tip 444 to protrude to a radius that is the same as or less than the radius of the outer frame 306. Axial clearance is present between the tip 444 and the outer frame 306. The outer frame 306 does not impede the tip 444 from being positioned at or less than the radius of the outer frame 306.

A configuration as shown in FIG. 67 may be provided in a variety of manners. In examples, a relative axial height of the distal end of the outer frame 306 and the tip 444 may be offset such that the tip 444 is positioned distal of the outer frame 306. In examples, the axial height of the anchors 442 may be reduced from the height of the anchors 304 shown in FIG. 49. The reduced axial height allows the tip 444 to tuck into or nest beneath or distal of the struts of the outer frame 306. In examples, the axial height of the outer frame 306 may be adjusted to position the distal end of the outer frame 306 proximal of the tips 444 to allow the tips 444 to tuck or nest distal of the struts of the outer frame 306.

A reduced radial extent or outer profile of the prosthetic valve 440 may result as compared to the prosthetic valve 280 shown in FIG. 49. FIG. 68 illustrates a top view of the prosthetic valve 440 illustrating that the tips 444 protrude no further than the outer surface of the outer frame 306. A reduced outer profile or outer diameter of the prosthetic valve results. A reduced force directed radially outward may result.

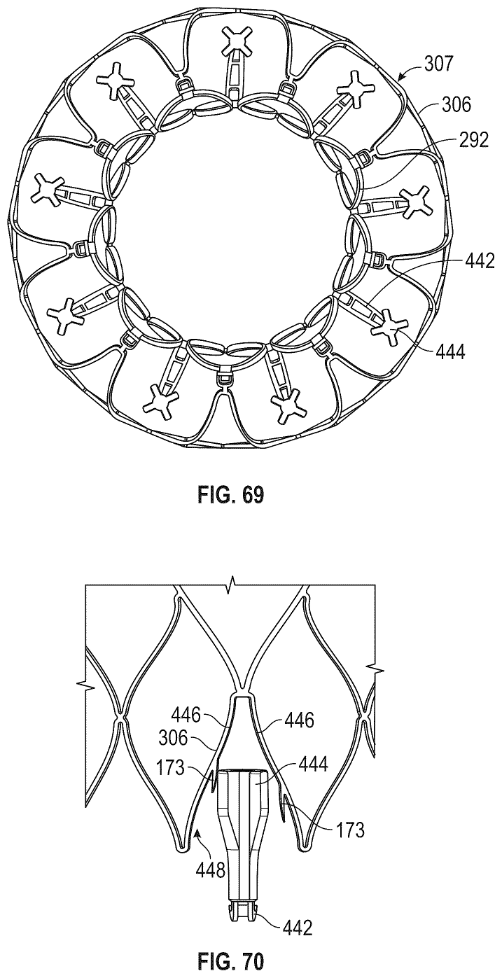

FIG. 69 illustrates a top view of the outer frame 306 and inner frame 292 with the tips 444 of the anchors 442 having the reduced height shown in FIG. 67. The tips 444 are positioned radially inward of the outer surface 307 of the outer frame 306. The tips 444 may be positioned radially inward of the outer surface 307 in examples, or may be at the same radius. For example, the outer frame 306 may be compressed radially inward by a skirt as disclosed herein, yet the tips 444 may remain at or lesser than the outer radius of the outer frame 306 as shown in FIG. 67.

FIG. 70 illustrates a side view of the tip 444 positioned distal of the outer frame 306. The tip 444 is positioned distal of the distalmost struts 446 of the outer frame 306. The distalmost struts 446 may extend axially and bound an opening 448 or distalmost opening positioned between the adjacent distalmost struts 446. The tip 444 may be positioned within the opening 448, in a configuration as shown and discussed regard to FIG. 28. In examples, the tip 444 may be positioned distal of the opening 448.

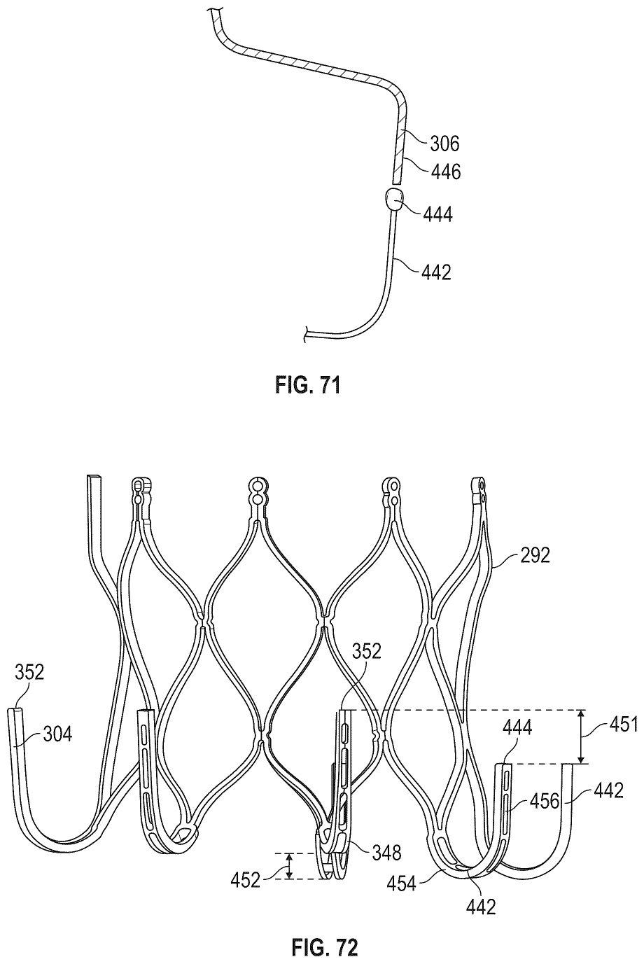

FIG. 71 illustrates a side cross sectional schematic view of a configuration as shown in FIG. 70. A skirt (e.g., the sealing skirt 308) may be positioned radially inward of the tip 444 as disclosed herein. A native valve leaflet may be positioned between the tip 444 and the skirt, and may be clamped in position. In examples, grip features as disclosed herein (e.g., as disclosed in FIGS. 19-31 and 52, or other forms of grip features) may be utilized for additional securement of the native valve leaflet. FIG. 70, for example, illustrates grip features 173 positioned proximate the tip 444, in a configuration shown in FIG. 28 for example. The grip features 173 may be utilized in manners disclosed herein.

In examples, the tip 444 may be partially recessed or at least partially recessed relative to the outer surface 307 of the outer frame 306.

The features of the prosthetic valve of FIGS. 67-71 may be utilized solely or in combination with any other example disclosed herein.

FIGS. 72-75 illustrate a variation in which a portion of the anchors of a prosthetic valve 450 comprise the anchors 442 discussed in regard to FIGS. 67-71, and a portion of the anchors comprise the anchors 304 of the prosthetic valve 280. The anchors 304 each have a tip 352 positioned radially outward of the outer frame 306 and that overlaps the outer surface 307 of the outer frame 306. Such a configuration is represented in FIG. 26, for example. The anchors 442 each have a tip 444 positioned distal of the outer frame 306, as discussed in regard to FIGS. 67-71. The anchors 442 may each protrude to a radius that is the same or less than a radius of the outer surface 307 of the outer frame 306. The anchors 442 have tips that are at least partially recessed radially inward of the outer surface of the prosthetic valve. Other features of the anchors 442 discussed in regard to FIGS. 67-71 may be utilized.

The anchors 304, 442 may be coupled to the inner frame 292 in a configuration represented in FIG. 72. As such, the heights of the anchors 304, 442 may be offset such that the tip 444 of the anchor 442 extends to a lesser axial height 451 in a proximal direction (or inflow direction) than the tip 352 of the anchor 304.

In examples, the offset in the heights of the tips 352, 444 may be produced due to a height offset 452 between the drop loop 348 of the anchor 304 and a drop loop 454 of the anchor 442. The drop loop 454 of the anchor 442 protrudes axially in a distal direction relative to a position of the drop loop 348 of the anchor 304. Such a feature extends the anchor 442 in a distal direction (or outflow direction) to reduce the height of the tip 444. As such, a corresponding reduction in the height of the tip 444 without a corresponding or equal reduction in the length of the tip portion 456 results.

In examples, at least one of the anchors of the prosthetic valve may comprise the anchor 304, and at least one of the anchors may comprise the anchor 442. In examples, the anchor 442 may be positioned in a desired location about the outer circumference of the prosthetic valve to provide a reduced radial extent or outer profile of the prosthetic valve at that position.

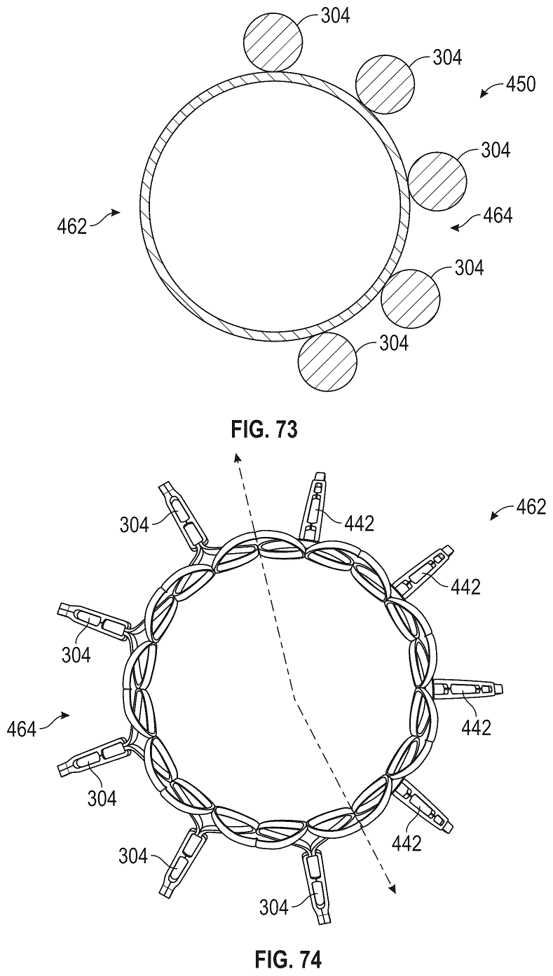

For example, referring to FIG. 73, a schematic top view of the prosthetic valve 450 is illustrated, with a portion 462 of the outer circumference including the anchor 442 and a portion 464 including one or more of the anchors 304. The portion 462, in examples, may be positioned to reduce the radial extent or outer profile of the prosthetic valve 450 at that portion 462. In examples in which the prosthetic valve 450 comprises a prosthetic tricuspid valve, the portion 462 may comprise a septal side of the prosthetic valve 450 adapted to face towards the septal side of the native tricuspid valve. As such, reduced radial pressure and possible electrical conduction disturbances with the tricuspid valve may occur at the portion 462. Other configurations may be utilized in examples.

The relative size of the portions 462, 464 may vary in examples. As represented in FIGS. 73 and 74, the portion 464 may comprise at least 200 degrees of the outer circumference of the prosthetic valve 450. The portion 462 accordingly may comprise 160 degrees or less. In examples, the portion 464 may comprise at least 180 degrees of the outer circumference. The portion 462 may comprise 180 degrees or less. Various other proportions (e.g., greater or lesser sizes of the portions 462, 464) may be utilized in examples. The portions 462, 464 may comprise arcs of the total outer circumference of the prosthetic valve 450. The portions 462, 464 are circumferentially adjacent to each other about the outer circumference of the prosthetic valve 450.

In examples, the portion 464 may include at least two of the anchors 304, or at least three of the anchors 304, or other amounts (e.g., at least four, five, etc.). The portion 462 may include at least two of the anchors 442, or at least three of the anchors 442, or other amounts (e.g., at least four, etc.). FIG. 74, for example, illustrates a configuration of the anchors 304, 442 in which the portion 464 includes five anchors 304 and the portion 462 includes four anchors 442. The anchors 304, 442 may each be circumferentially spaced equally from each other. In examples, unequal circumferential spacing may be utilized.

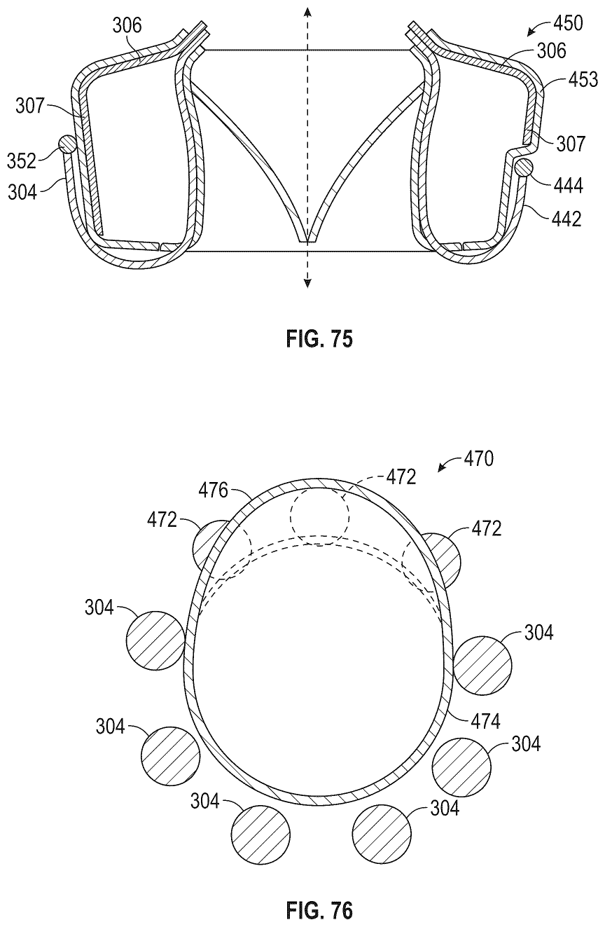

FIG. 75 illustrates a cross sectional view of the prosthetic valve 450, illustrating the use of both anchors 304 and anchors 442. An asymmetric configuration of anchors 304, 442 results, with a portion 462 of the prosthetic valve 450 utilizing anchors 442 and a portion 464 utilizing anchors 304. In examples, the anchors 304, 442 may compress against grip features as disclosed herein (e.g., as disclosed in FIGS. 19-31 and 52, or other forms of grip features). The grip features may be utilized to secure the leaflet in the space between the respective tip of the anchor and the outer frame 306. For example, a tip 352 of the anchor 304 may overlap a grip feature to press a native valve leaflet towards the grip feature to reduce movement of the leaflet relative to the valve body 453.

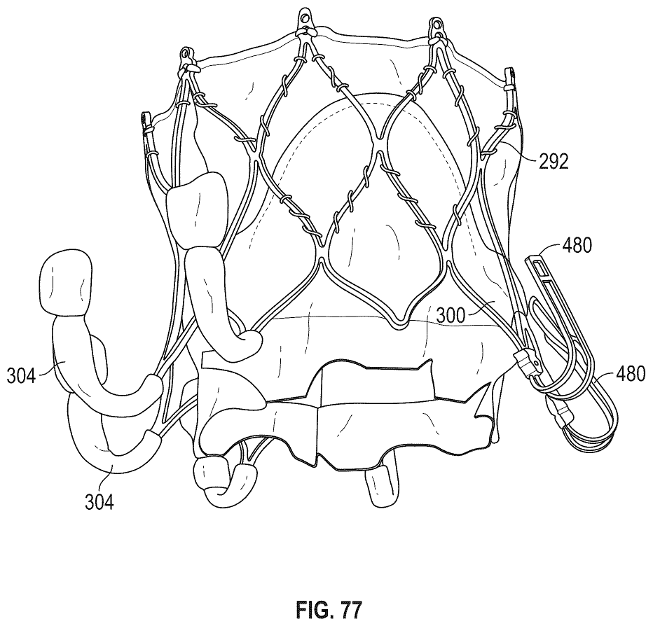

The features of the prosthetic valve of FIGS. 72-75 may be utilized solely or in combination with any other example disclosed herein. Alternative configurations may be provided such that the anchors utilized have a same or lesser radius than an outer surface of the outer body, or that the anchors are tucked or nested within the outer body. FIG. 76, for example, illustrates a top schematic view of a configuration of a prosthetic valve 470 in which anchors 304 are utilized, and anchors 472 are utilized that have a lesser axial height than the anchors 304. The anchors 472 each have a height set such that the tips of the anchors 472 are distal of the outer frame 474 of the prosthetic valve 470. The outer frame 474 has an ovalized outer profile (or non-uniformly circular outer profile) such that a portion 476 of the outer frame 474 extends over the anchors 472 fully or partially. As such, a reduced radial extent of the anchors 472 from the outer surface of the outer frame 474 results. The outer frame 474 may extend outward with a large diameter portion 476 for at least one-third of the prosthetic valve 470. Other proportions may be utilized in examples. The portion 476 may have wider strut openings in examples to accommodate the tips of any of the anchors disclosed herein.

The features of the prosthetic valve of FIG. 76 may be utilized solely or in combination with any other example disclosed herein.

Additional variations in the configuration of the prosthetic valve 280 may be provided. FIGS. 77-86, for example, illustrate a variation in which one or more clasp anchors 480 may be utilized. FIG. 77 illustrates a side perspective view of the inner frame 292 showing clasp anchors 480 coupled to the distal end portion 300 of the inner frame 292. The anchors 304 may be coupled to the inner frame 292.

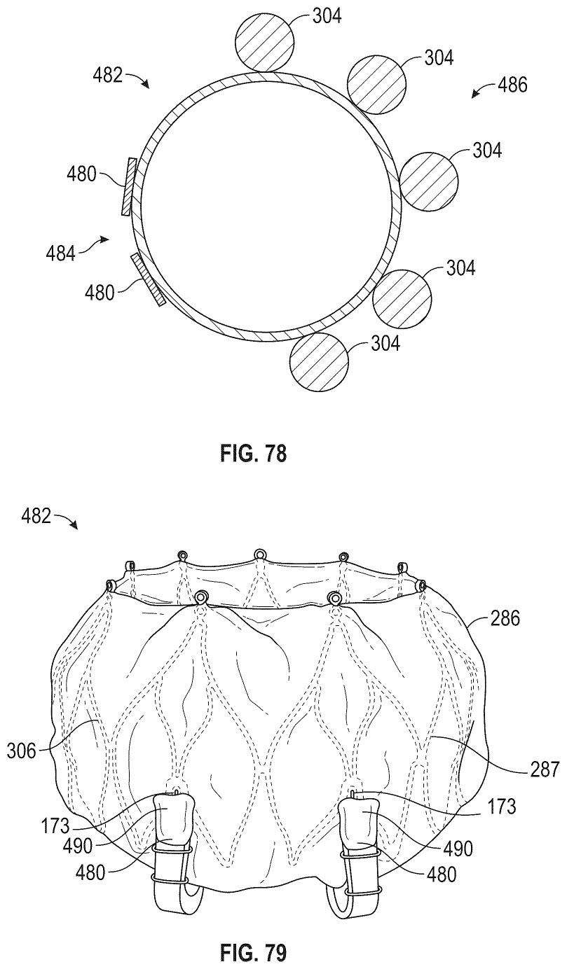

A prosthetic valve 482 (marked in FIG. 79) utilizing the one or more clasp anchors 480 may be divided into portions in a similar manner as disclosed in regard to FIGS. 73 and 74. For example, referring to the top schematic view of FIG. 78, the outer circumference of the prosthetic valve 482 may be divided into a portion 484 including the clasp anchors 480 and a portion 486 including one or more of the anchors 304. The portion 484 may be opposite the portion 486. The portion 484, in examples, may be positioned to reduce the radial extent or outer profile of the prosthetic valve at that portion 484. In examples in which the prosthetic valve 482 comprises a prosthetic tricuspid valve, the portion 484 may comprise a septal side of the prosthetic valve 482 adapted to face towards the septal side of the native tricuspid valve. As such, reduced radial pressure and possible electrical conduction disturbances with the tricuspid valve may occur at the portion 484. Other configurations may be utilized in examples.

The relative size of the portions 484, 486 may vary in examples. As represented in FIG. 78, the portion 486 may comprise at least 200 degrees of the outer circumference. The portion 484 accordingly may comprise 160 degrees or less. In examples, the portion 486 may comprise at least 180 degrees of the outer circumference. The portion 484 may comprise 180 degrees or less. Various other proportions (e.g., greater or lesser sizes of the portions 484, 486) may be utilized in examples. The portions 484, 486 may comprise arcs of the total outer circumference of the prosthetic valve 482. The portions 484, 486 are circumferentially adjacent to each other about the outer circumference of the prosthetic valve 482.

In examples, the portion 486 may include at least two of the anchors 304, or at least three of the anchors 304, or other amounts (e.g., at least four, five, etc.). The portion 484 may include at least two of the clasp anchors 480, or at least three of the clasp anchors 480, or other amounts (e.g., at least four, etc.). FIG. 78, for example, illustrates a configuration of the anchors 304, 442 in which the portion 486 includes five anchors 304 and the portion 484 includes two clasp anchors 480. The anchors 304 are shown spaced equally with each other, and the clasp anchors 480 are shown spaced equally from each other. In examples, unequal circumferential spacing may be utilized.

FIG. 79 illustrates a perspective view of the prosthetic valve 482. The clasp anchors 480 are coupled to the valve body 286. The clasp anchors 480 each include tips 490 that are adapted to overlap the outer surface 307 of the outer frame 306 and are adapted to clasp a portion of the native valve (e.g., the native valve leaflet) between the tip 490 and the outer surface 307 of the outer frame 306 to anchor to the native valve. The clasp anchors 480 are adapted to clasp the native valve leaflet against the outer surface 287 of the valve body 286. The clasp anchors 480 are spring biased radially inward toward the center of the prosthetic heart valve.

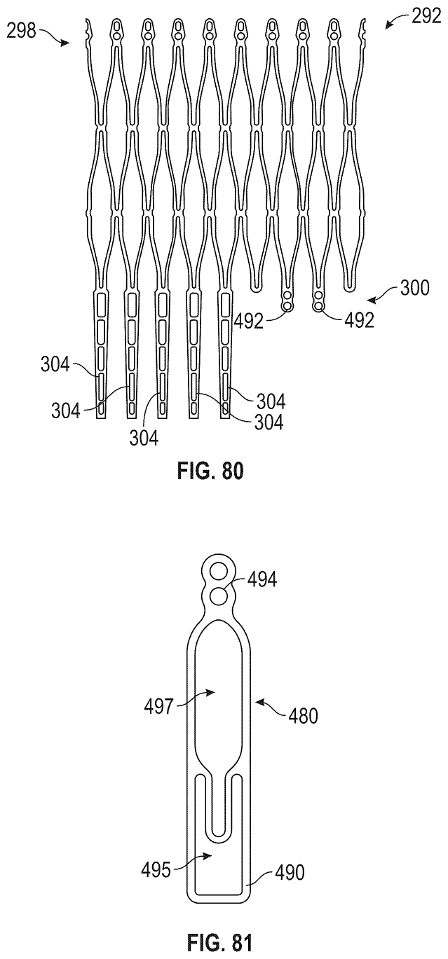

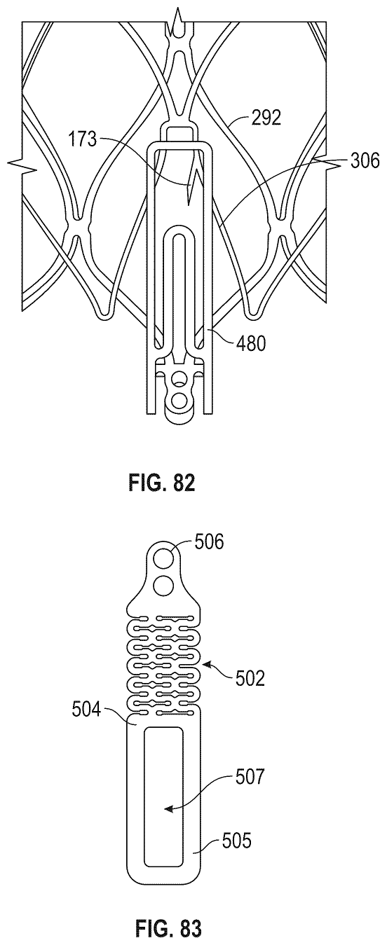

FIG. 80 illustrates a flat pattern of the inner frame 292 showing the arms of the anchors 304 extending distally from the distal end portion 300 of the inner frame 292 in an elongate configuration. The distal end portion 300 includes couplers 492 for coupling with the clasp anchors 480. FIG. 81 illustrates a plan view of an arm of a clasp anchor 480. The clasp anchor 480 extends longitudinally to the tip 490 of the clasp anchor 480. The clasp anchor 480 includes a coupler 494 for coupling with the coupler 492 of the inner frame 292. The clasp anchor 480 may including windows or openings 495, 497 for reducing the material of the clasp anchor arm and for receiving heart leaflet tissue therein if desired. The couplers 492, 494 may couple utilizing a suture coupling or other form of coupling as desired.

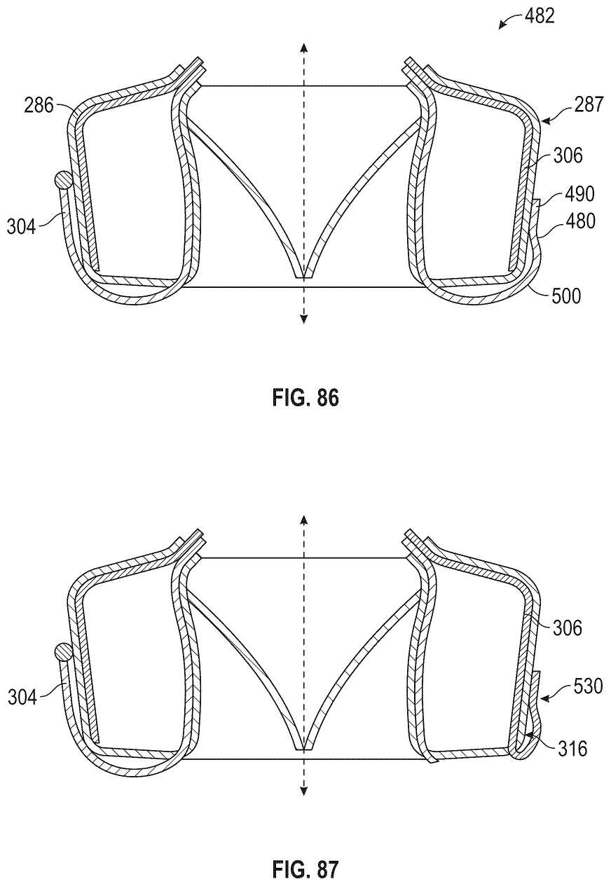

The clasp anchor 480 may be shape set to curve towards the outer frame 306. Referring to FIG. 82, for example, the clasp anchor 480 may be shape set to curve in a proximal direction and accordingly press radially inward towards the outer frame 306. The clasp anchor 480 may overlap the outer frame 306 as shown in FIG. 82. The deflection of the clasp anchor 480 may produce a drop loop 500 as shown in the cross sectional view of FIG. 86. The drop loop 500 may produce a spring bias, which may be similar to the bias of a torsion spring, to direct the tip 490 towards and against the outer frame 306. The clasp anchor 480 may be spring biased towards the valve body 286.

In examples, the tip 490 may have a circumferentially planar shape, such that a flat profile is produced against the outer surface of the valve body 286. The tips 490 accordingly may be positioned flush against the outer surface of the valve body 286 as represented in the schematic view of FIG. 78. A reduced outer profile of the prosthetic valve 482 accordingly may result.

The configuration of the clasp anchor 480 may be varied in examples. FIG. 83, for example, illustrates a variation in which a clasp anchor 504 includes a flexible portion 502 for enhancing a flexibility of the anchor 504 at that portion 502. The flexible portion 502 may comprise an undulating pattern or rachis pattern of the arm at that portion 502. The clasp anchor 504 may otherwise be configured similarly as the clasp anchor 480, and may include a tip 505 and a coupler 506 for coupling to the coupler 492. A window or opening 507 may be positioned at the tip 505.

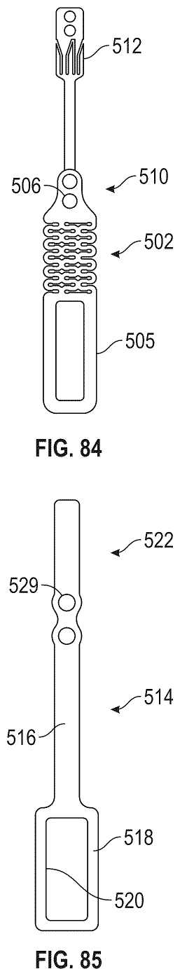

FIG. 84 illustrates a variation in which a clasp anchor 510 is configured similarly as the clasp anchor 504, yet includes an additional barbed clasp 512 that may be positioned in the location of the grip features 173 shown in FIG. 82. The barbed clasp 512 may be positioned to engage the native valve leaflet upon closure of the clasp anchor 510.

FIG. 85 illustrates a linear configuration of a clasp anchor 514 comprising an arm 516 with a tip 518 having a window or opening 520. The clasp anchor 514 includes a top clasp 522 and a coupler 524 for coupling to the coupler 492.

FIG. 86 illustrates a cross sectional view of the prosthetic valve 482 illustrating the clasp anchor 480 positioned against the outer surface 287 of the valve body 286.

Variations in the coupling of the clasp anchor 480 may be provided. FIG. 87, for example, illustrates a variation in which a clasp anchor 530, configured similarly as the clasp anchor 480, is coupled to the outer frame 306. The clasp anchor 530 may be coupled to the distal end portion 316 of the outer frame 306.

Further, in examples, grip features as disclosed herein (e.g., as disclosed in FIGS. 19-31 and 52, or other forms of grip features) may be utilized to secure the leaflet in the space between any of the clasp anchors and the anchors 304 and the outer frame 306 in manner disclosed herein. The clasp anchors or anchors 304 may overlap the outer frame 306 having grip features or clamping may occur in other manners.

Further variations may be provided of any example of prosthetic valve disclosed herein. In examples, any of the anchors disclosed herein (which may include hook arm anchors or clasp anchors) may be excluded at a portion of any prosthetic valve such that unequal spacing of the remaining anchors results. One or more anchors may be excluded along a circumference of the prosthetic valve. The anchors may be excluded on a septal portion of a prosthetic valve in a tricuspid deployment. Portions of a prosthetic valve disclosed herein as having at least partially recessed anchors or as having clasp anchors may exclude anchors entirely in examples.

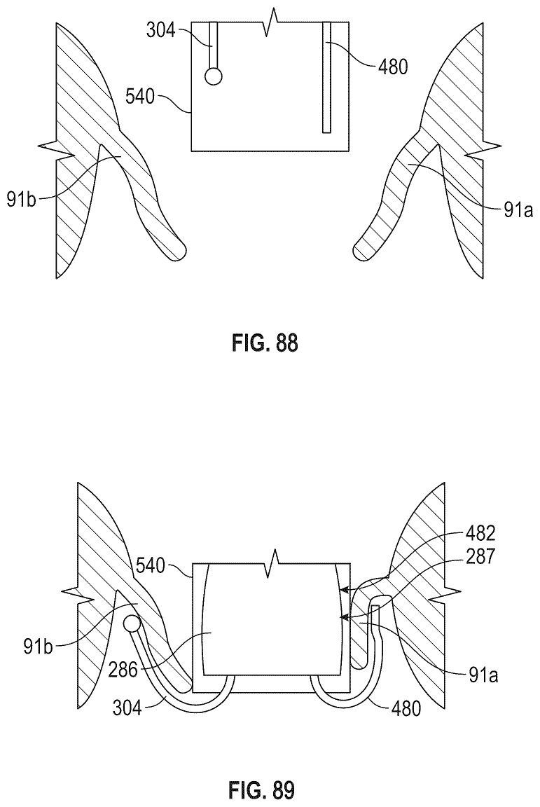

FIGS. 88-90 illustrate an exemplary deployment sequence of the prosthetic valve 482 to a native heart valve. FIG. 88 illustrates the anchors 304, 480 each in a linearized, extended, or compressed configuration within a capsule 540 of a delivery system or delivery catheter. The clasp anchor 480 may have a greater length than the anchor 304 in the linearized, extended, or compressed configuration. The capsule 540 approaches a native heart valve, which may be a native tricuspid valve in examples. The native heart valve may include the leaflets 91a, b.

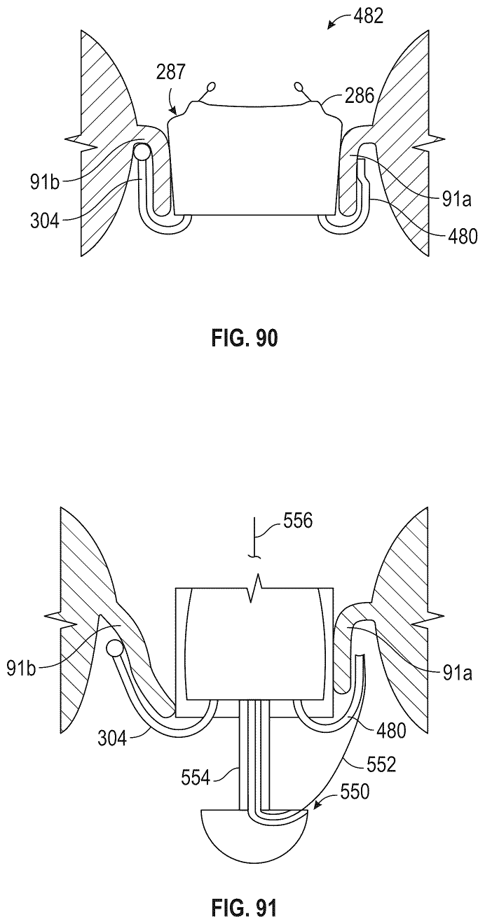

FIG. 89 illustrates the prosthetic heart valve 482 partially deployed, in which the anchor 304 is partially deployed, and the clasp anchor 480 extends radially outward from the capsule 540. The clasp anchor 480 may capture a native valve leaflet 91a and may press the leaflet 91a against the outer surface of the capsule 540 in the partially deployed configuration. The capsule 540 may be retracted from this position to allow the clasp anchor 480 to press the leaflet 91a against the outer surface 287 of the valve body 286 as represented in FIG. 90 for example.

FIG. 90 illustrates a deployed configuration of the prosthetic heart valve 482, with the clasp anchor 480 clasping the leaflet 91a against the outer surface 287 of the valve body 286.

Variations in the delivery system may be provided. FIG. 91, for example, illustrates a variation in which a delivery system 550 includes a tether 552 coupled to the clasp anchor 480 for controlling closing and/or opening of the clasp anchor 480. The tether 552 may be tensioned to open the clasp anchor 480 and tension may be released to allow the clasp anchor 480 to close. As such, control of the opening and closing of the clasp anchor 480 may be provided.

The tether 552 may extend to a shaft of the delivery system 550 such as a guide wire lumen 554 or other shaft of the delivery system 550. The tether 552 may pass through a channel in the shaft to a proximal end portion of the delivery system 550 for actuation. A proximal end portion 556 of the tether 552 may be actuated at a handle or other ex vivo portion of a delivery system 550.

In examples, the prosthetic heart valve 482 may include only clasp anchors (and lacking hook arm anchors) distributed circumferentially about the prosthetic heart valve 482.

The features of the examples of FIGS. 77-91 may be utilized solely or in combination with any other example disclosed herein.

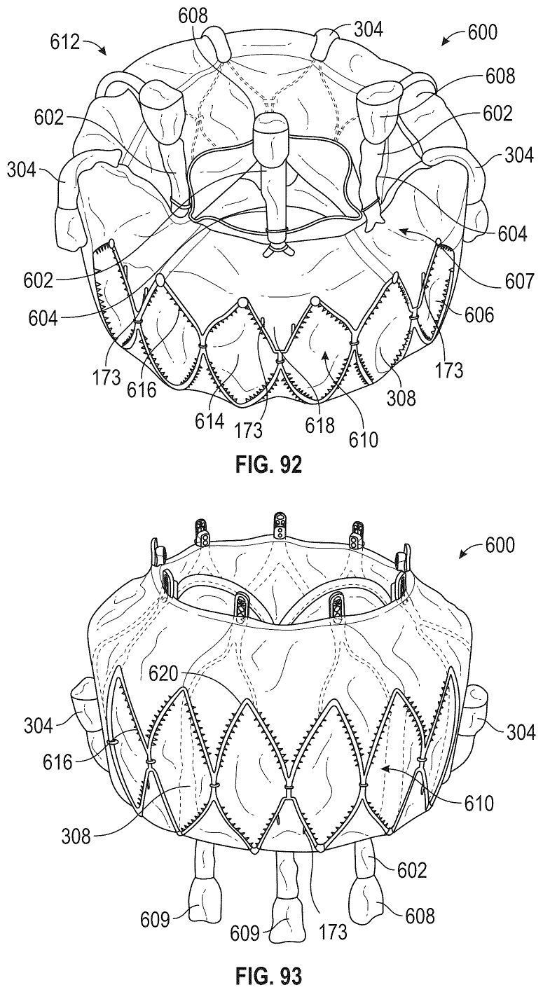

Additional variations in the configuration of the prosthetic valve 280 may be provided. FIGS. 92-96 illustrate a variation in which a prosthetic valve 600 includes one or more support arms 602. The prosthetic valve 600 may include the features of the prosthetic valve 280 unless stated otherwise. The support arms 602 may each have a proximal end portion 604 coupled to the valve body 606 and a distal end portion 608 protruding in an axially distal or outflow direction from the valve body 606. The support arms 602 may protrude from the distal end portion 607 of the valve body 606. The support arms 602 may extend in the distal or outflow direction to a tip 609 of the respective support arm 602.

The support arms 602 may be positioned at a portion 610 of the prosthetic valve 600. The prosthetic valve 600 may be divided into portions in a similar manner as discussed in regard to FIGS. 73 and 78. For example, the prosthetic valve 600 may include the portion 610 and a portion 612 including the anchors 304. The portions 610, 612 may be opposite each other. The relative size of the portions 610, 612 may vary in examples. For example, the portion 612 may comprise at least 200 degrees of the outer circumference of the prosthetic valve 600. The portion 610 accordingly may comprise 160 degrees or less. In examples, the portion 612 may comprise at least 180 degrees of the outer circumference. The portion 610 may comprise 180 degrees or less. Various other proportions (e.g., greater or lesser sizes of the portions 610, 612) may be utilized in examples. The portions 610, 612 may comprise arcs of the total outer circumference of the prosthetic valve 600. The portions 610, 612 are circumferentially adjacent to each other about the outer circumference of the prosthetic valve 600.

The support arms 602 may extend at the circumferential portion 610. The prosthetic valve 600 may lack hook arm anchors protruding radially outward from the outer surface of the valve body at the portion 610. The numbers of support arms 602 may vary from at least one, to at least two, or at least three, or a greater number. The anchors 304 may include at least one, at least two, at least three, or a greater number (e.g., six are shown in FIG. 92).

In a tricuspid deployment, the portion 610 may be set to be positioned at a septal side of the tricuspid valve. As such, a reduced radial profile may be produced at the septal side of the tricuspid valve, to reduce the possibility of electrical conduction disturbances at the tricuspid valve.

In a deployment to a native valve between a heart atrium and a heart ventricle, the support arms 602 may be adapted to extend into the ventricle. The support arms 602 may be adapted to stabilize the prosthetic valve 600 within the native valve. The support arms 602, for example, may contact the heart wall within the ventricle to stabilize the prosthetic valve 600 or otherwise may be positioned to stabilize the prosthetic valve 600. The support arms 602 may reduce tilt or rocking of the prosthetic valve 600 during pulsatile movement of the heart and due to the lack of hook anchors at the portion 610. As such, the support arms 602 may account for the lack of hook anchors at the portion 610.

The support arms 602 may extend from the inner frame 292 (as shown in FIG. 92) or may extend from the outer frame 306 in examples. The support arms 602, for example, may be coupled to the distal end portion of the inner frame 292 or the distal end portion of the outer frame 306.

The support arms 602, in examples, may be composed with the structure of the anchors 304, yet shape set to protrude in the distal direction or outflow direction. The support arms 602 accordingly may comprise the anchors 304 that are not shape set to form hooks, but are rather shape set to extend axially or linearly. Other configurations of support arms 602 may be utilized in examples.

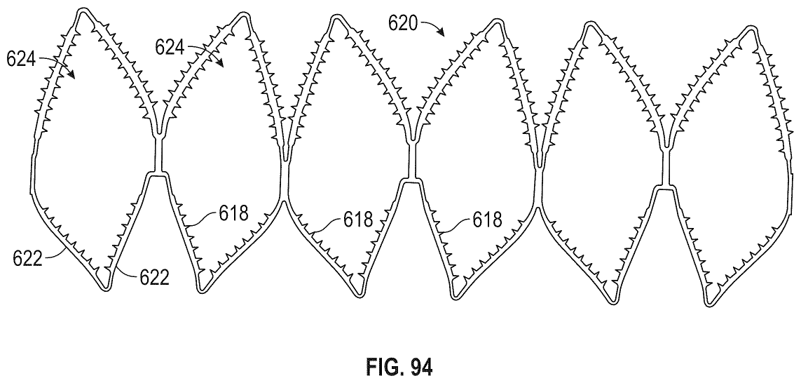

In examples, the outer surface 614 of the valve body 606 may include a friction element 616. The friction element 616 is adapted to provide friction with the native heart valve, for example, with the native valve leaflets or the annulus. The friction element 616 as shown in FIGS. 92 and 93 comprises a plurality of barbs 618, which may be positioned on a sheet 620 of material. The barbed sheet 620 is shown in isolation in FIG. 94. The sheet 620 may include a plurality of arms 622 bounding openings 624. The barbs 618 may be positioned on the arms 622. The barbs 618 may be flattened or coplanar with the sheet 620 in examples, such that the barbs 618 do not protrude radially outward from the sheet 620. The barbs 618 may extend parallel with the plane of the outer surface of the valve body 606. The barbs 618 being coplanar with the sheet 620 may reduce the possibility of damage to a deployment capsule due to the barbs 618 upon release of the prosthetic valve 600 from the capsule. In examples, the barbs 618 may be configured to protrude radially outward from the sheet 620 or other configurations may be provided. The arms 622 may be shaped to match the shape of the struts of the outer frame 306 in examples.

The sheet 620 may be positioned on the outer surface 614 of the valve body 606 as shown in FIGS. 92 and 93. In examples, the sheet 620 may be positioned interior of the skirt 308 and the barbs 618 may be adapted to protrude through the skirt 308.

Other forms of friction elements may be utilized in examples, such as a roughened surface of the outer frame or outer skirt, a fuzzy textile, or other forms of barbs, among others.

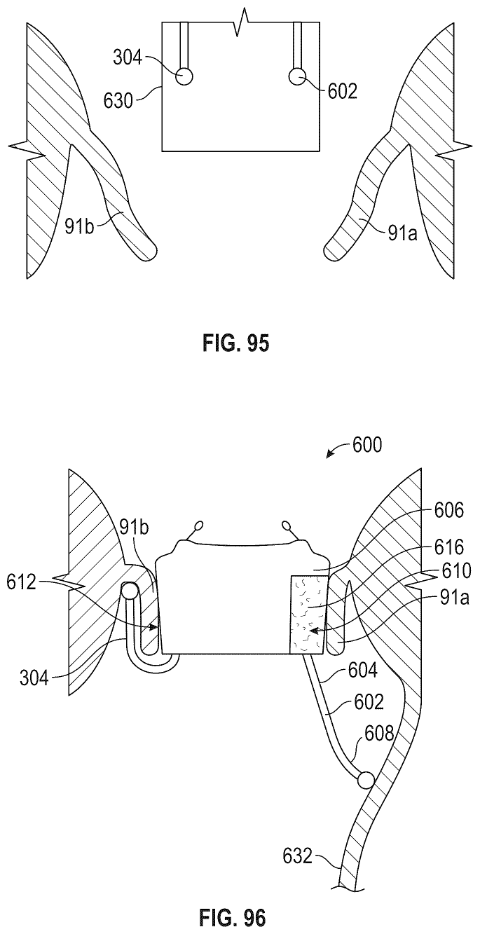

FIGS. 95 and 96 illustrate an exemplary deployment sequence. Referring to FIG. 95, the anchors 304 are in a linearized, extended, or compressed configuration, and the support arms 602 are shown extended within a delivery capsule 630 as well. The delivery capsule may retract in a similar manner as disclosed herein, with the anchors 304 flipping or rotating to hook over the leaflet 91b. The support arm 602 may continue to protrude axially due to its shape set.

Referring to FIG. 96 for example, the anchor 304 hooks over the leaflet 91b while the support arm 602 protrudes axially into the ventricle for stabilization of the prosthetic valve 600. The support arm 602 may be positioned proximate or may contact the heart wall 632 of the ventricle. The support arm 602 may contact the inner heart wall 632 within the ventricle to stabilize the prosthetic valve 600 within the tricuspid valve. Tilt of the prosthetic valve 600 may be reduced by the presence of the support arm 602. The friction element 616 may engage the leaflet 91a and/or the annulus to secure the portion 610 of the prosthetic valve 600 in position. A reduced possibility of atrial migration and embolism may result.

In examples, transjugular entry of the prosthetic valve 600 may be utilized.

The features of the examples of FIGS. 92-96 may be utilized solely or in combination with any other example disclosed herein.

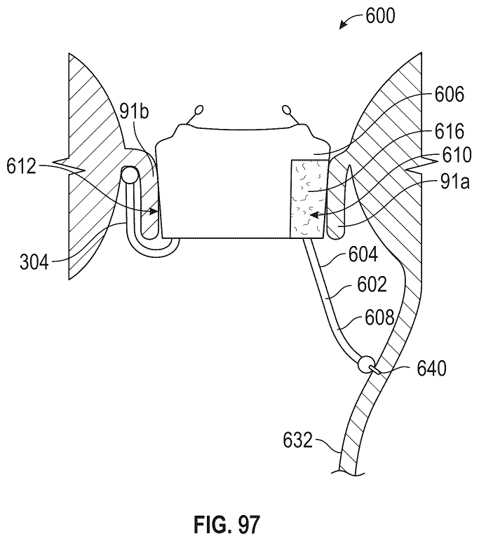

FIG. 97 illustrates a variation in which the support arm 602 includes a puncturing element 640 for puncturing a heart wall of the ventricle to secure the support arm 602 to the heart wall. The puncturing element 640 may comprise a barb, prong, or screw tip, or other form of puncturing element for puncturing tissue. The puncturing element 640 may be at the tip of the support arm 602. During deployment, the puncturing element 640 may be penetrated into the tissue of the heart wall.

The examples of prosthetic valves may be utilized in a mitral valve or tricuspid valve as disclosed herein, or may be utilized in other deployment locations. The prosthetic heart valve may be sized for replacing a native tricuspid or mitral valve. Deployment to aortic or pulmonary valves, or other implantation sites may be utilized.

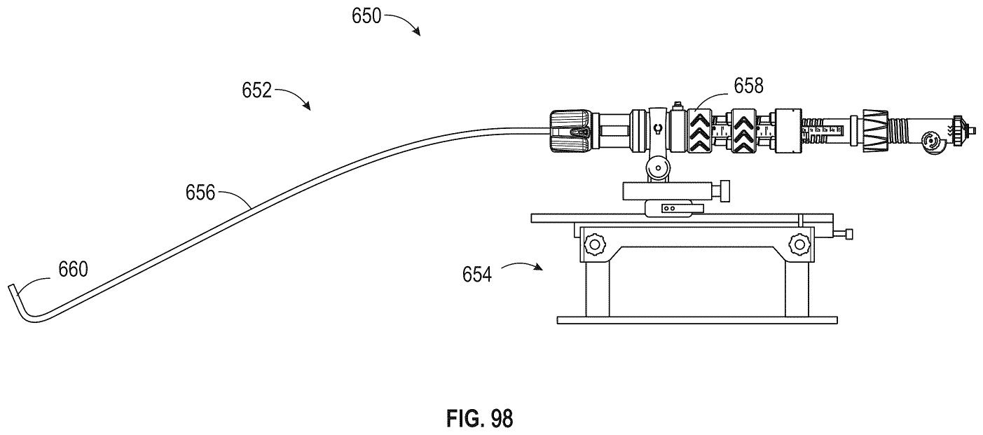

FIG. 98 illustrates an example of a delivery system 650. Features of a delivery system 650 that may be utilized are disclosed in U.S. Provisional Application No. 63/436,051, filed Dec. 29, 2022, and U.S. Provisional Application No. 63/533,458, filed Aug. 18, 2023 the entire contents of each of which are hereby incorporated by reference. The delivery system 650 can be used to deploy an implant as disclosed herein, or another form of implant. An implant such as a prosthetic heart valve may be delivered to a subject's mitral or tricuspid valve annulus or other heart valve location in various manners, such as by open surgery, minimally-invasive surgery, and percutaneous or transcatheter delivery through the subject's vasculature. Example transfemoral approaches are described further in U.S. Pat. Publ. No. 2015/0238315, published Aug. 27, 2015, the entirety of which is hereby incorporated by reference in its entirety. While the delivery system 650 is described in connection with a percutaneous delivery approach, and more specifically a transfemoral delivery approach, it should be understood that features of the delivery system 650 can be applied to other delivery approaches, including delivery systems for a transapical or transjugular delivery approach.

The delivery system 650 may be used to deploy a prosthesis, such as a replacement heart valve as described in this specification, to a location within the body of a subject. The delivery system 650 may include multiple components, devices, or subassemblies. The delivery system 650 may include a delivery catheter or elongate catheter or delivery device 652, and a stabilizer assembly 654, and other components as desired. The delivery device 652 may include an elongate shaft or shaft assembly 656 and a housing in the form of a handle 658. The housing may be at the proximal end portion of the elongate shaft or shaft assembly 656. The shaft assembly 656 may include one or more shafts. A plurality of shafts may be provided according to examples herein, although in examples a single shaft may be utilized. The shaft assembly 656 may include a capsule 660 that may retain the implant in a compressed configuration, with the capsule 660 retracted to deploy the implant. In examples, sutures may couple to the implant that are utilized to release the implant at the implantation site. A tether assembly, for example, may be utilized for release.

Percutaneous and transseptal implantation may be utilized. Implantation to a tricuspid valve may be utilized.

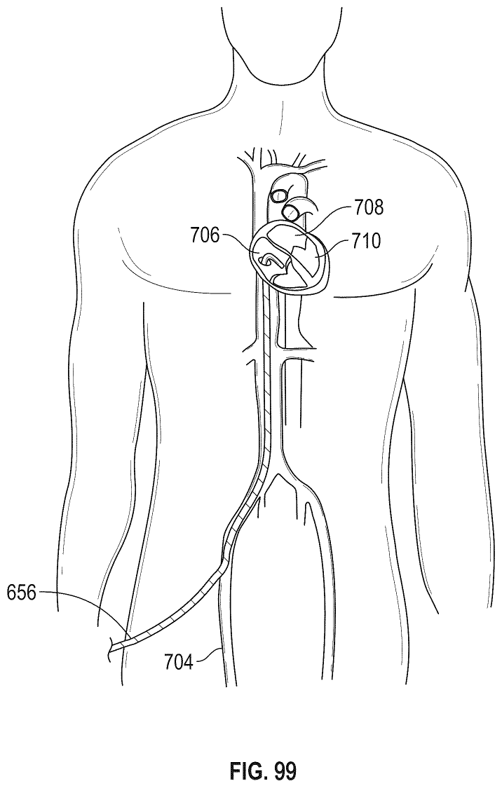

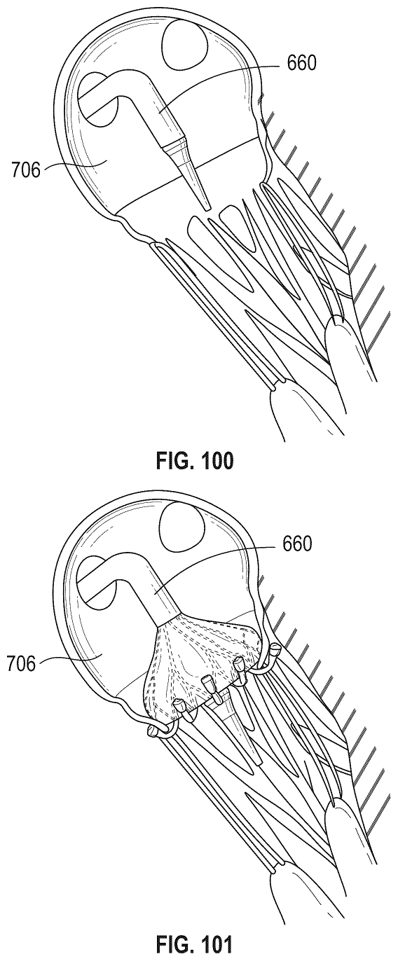

FIG. 99 illustrates a schematic representation of a delivery approach to a native tricuspid valve. As shown in FIG. 99, in one example the delivery system can be placed in the ipsilateral femoral vein 704 and advanced toward the right atrium 706. The approach can be from the inferior vena cava (or from the superior vena cava) in examples.

FIG. 99 shows the delivery system extending from the ipsilateral femoral vein 704 to the right atrium 706. In examples of the disclosure, a guide wire is not necessary to position the delivery system in the proper position, although in other examples, one or more guide wires may be used.

Accordingly, it can be advantageous for a user to be able to steer the delivery system through the complex areas of the heart in order to position a replacement tricuspid valve in line with the native tricuspid valve. This task can be performed with or without the use of a guide wire with the above disclosed system. The distal end of the delivery system can be advanced into the right atrium 706. A user can then manipulate the delivery system to target the distal end of the delivery system to the appropriate area. Further, a user can torque the entire delivery system to further manipulate and control the position of the delivery system. In the fully bent configuration, a user can then place the replacement valve in the proper location. This can advantageously allow delivery of a replacement valve to an in-situ implantation site, such as a native tricuspid valve.

FIG. 100 illustrates a schematic representation of a distal end of the delivery system approaching a native tricuspid valve. The distal end of the delivery system may be positioned as desired relative to the implantation site prior to release of an implant from the implant retention area.

FIG. 101 illustrates that the implant may be released from the delivery system with a tether assembly 662 coupled to the implant. The position of anchors relative to the native valve leaflets may be determined, and if in proper position the implantation procedure may proceed.

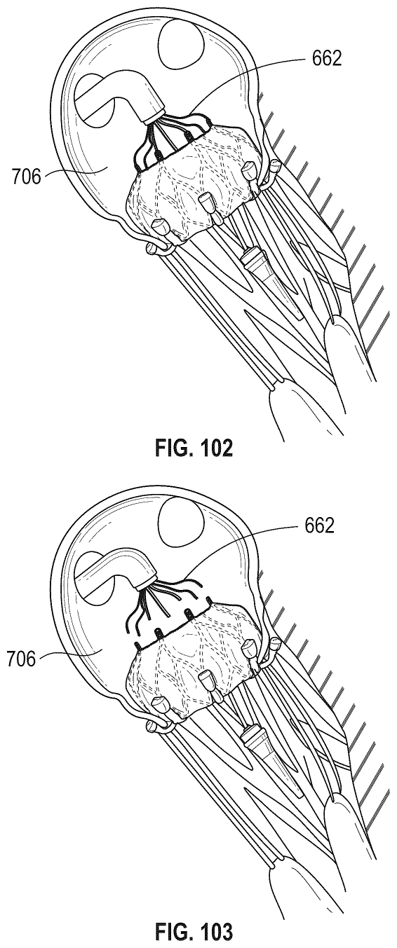

FIG. 102 illustrates the implant in an expanded configuration, with the tether assembly 662 coupled to the implant. The position of anchors relative to the native valve leaflets may be determined, and if in proper position the implantation procedure may proceed.

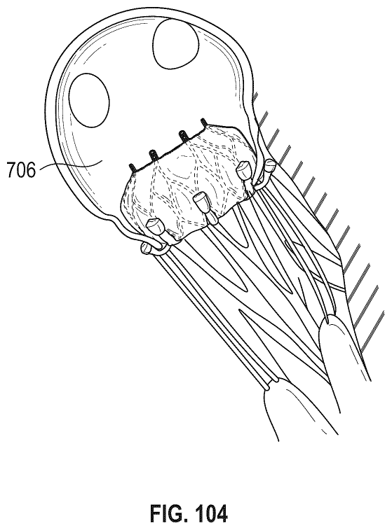

The tether assembly 662 may be released from the implant utilizing release of a release assembly. FIG. 103 illustrates release of the tether assembly 662. FIG. 104 illustrates the implant deployed in position.

A similar deployment procedure may be utilized with a mitral valve as desired. For example, a transseptal puncture can be performed from the right atrium 706 (marked in FIG. 99) to obtain access to the left atrium 708. A user can pass the bent delivery system through the transseptal puncture and into the left atrium 708. The delivery system can then be advanced into the left atrium 708 and then towards the left ventricle 710. The implant may be deployed in a similar manner as shown in FIGS. 99-104.

The delivery systems disclosed herein may be utilized with any example disclosed herein.

Various modifications of the examples disclosed herein may be provided. Features of examples may be modified, substituted, excluded, or combined across examples as desired. Combinations of features across examples may be provided as desired. Combinations of features may be provided across examples with other features of such examples being excluded if desired.

The various examples of sealing skirts disclosed herein may have a variety of forms, including cloth skirts, foam skirts, or braided skirts as desired. Various materials may be utilized as desired.

The implants disclosed herein may include prosthetic heart valves or other forms of implants, such as stents or filters, or diagnostic devices, among others. The implants may be expandable implants configured to move from a compressed or undeployed state to an expanded or deployed state. The implants may be compressible implants configured to be compressed inward to have a reduced outer profile and to move the implant to the compressed or undeployed state.

Various forms of delivery apparatuses may be utilized with the examples disclosed herein. The delivery apparatuses as disclosed herein may be utilized for aortic, mitral, tricuspid, and pulmonary replacement and repair as well. The delivery apparatuses may comprise delivery apparatuses for delivery of other forms of implants, such as stents or filters, or diagnostic devices, among others.

The implants and the systems disclosed herein may be used in transcatheter mitral or tricuspid implantation, as well as aortic valve implantation (TAVI) or replacement of other native heart valves (e.g., pulmonary valves). The delivery apparatuses and the systems disclosed herein may be utilized for transarterial access, including transfemoral access, to a patient's heart. The delivery apparatuses and systems may be utilized in transcatheter percutaneous procedures, including transarterial procedures, which may be transfemoral. Transapical procedures, among others, may also be utilized. Other procedures may be utilized as desired.

In addition, the methods herein are not limited to the methods specifically described, and may include methods of utilizing the systems and apparatuses disclosed herein. The steps of the methods may be modified, excluded, or added to, with systems, apparatuses, and methods disclosed herein. The examples disclosed herein may comprise systems for implantation within a human body in examples.

For purposes of this description, certain aspects, advantages, and novel features of the examples of this disclosure are described herein. The disclosed methods, apparatuses, and systems should not be construed as limiting in any way. Instead, the present disclosure is directed toward all novel and nonobvious features and aspects of the various disclosed examples, along and in various combinations and sub-combinations with one another. The methods, apparatuses, and systems are not limited to any specific aspect or feature or combination thereof, nor do the disclosed examples require that any one or more specific advantages be present or problems be solved. Features, elements, or combinations of one example can be combined into other examples herein.

Example 1: A prosthetic valve configured to be deployed to a native valve, the prosthetic valve comprising: one or more prosthetic valve leaflets; and a valve body supporting the one or more prosthetic valve leaflets, wherein at least a portion of the prosthetic valve includes a material configured to reduce tissue formation or thrombus formation along the portion of the prosthetic valve.

Example 2: The prosthetic valve of any example herein, in particular example 1, wherein the portion of the prosthetic valve comprises a skirt.

Example 3: The prosthetic valve of any example herein, in particular example 2, wherein the prosthetic valve includes a frame and the skirt is positioned upon the frame.

Example 4: The prosthetic valve of any example herein, in particular examples 1-3, wherein the one or more prosthetic valve leaflets are positioned within a flow channel of the prosthetic valve, and the prosthetic valve includes a valve body having an outer surface facing outward from the flow channel and an inner surface facing towards the flow channel, and the portion of the prosthetic valve comprises the inner surface of the valve body.

Example 5: The prosthetic valve of any example herein, in particular example 4, wherein the outer surface of the valve body is configured for tissue formation or thrombus formation with the outer surface of the valve body.

Example 6: The prosthetic valve of any example herein, in particular example 4 or example 5, wherein the outer surface includes an end portion, and the inner surface includes an end portion proximate the end portion of the outer surface, and the portion of the prosthetic valve comprises the end portion of the inner surface.

Example 7: The prosthetic valve of any example herein, in particular example 6, wherein the end portion of the inner surface is coupled to the end portion of the outer surface.

Example 8: The prosthetic valve of any example herein, in particular examples 1-7, wherein the portion of the prosthetic valve comprises a barrier layer adjacent to the one or more prosthetic valve leaflets.

Example 9: The prosthetic valve of any example herein, in particular example 8, wherein the barrier layer is configured to reduce spread of tissue formation or thrombus formation to the one or more prosthetic valve leaflets.

Example 10: The prosthetic valve of any example herein, in particular example 8 or example 9, wherein the one or more prosthetic valve leaflets are coupled to the barrier layer.

Example 11: The prosthetic valve of any example herein, in particular examples 1-10, wherein the prosthetic valve has a proximal end portion and a distal end portion, the proximal end portion being at an inflow of the prosthetic valve, and the distal end portion being at an outflow of the prosthetic valve, and the portion of the prosthetic valve is positioned at the proximal end portion of the prosthetic valve.

Example 12: The prosthetic valve of any example herein, in particular example 11, wherein the portion of the prosthetic valve comprises a skirt extending distally from a proximal rim of the prosthetic valve.

Example 13: The prosthetic valve of any example herein, in particular examples 1-12, wherein the one or more prosthetic valve leaflets are positioned within a flow channel of the prosthetic valve, and the portion of the prosthetic valve bounds the flow channel.

Example 14: The prosthetic valve of any example herein, in particular example 13, wherein the portion of the prosthetic valve comprises a sleeve bounding the flow channel.

Example 15: The prosthetic valve of any example herein, in particular examples 1-14, wherein the prosthetic valve is configured to be deployed to a native mitral valve or a native tricuspid valve.

Example 16: A method comprising: deploying a prosthetic valve to a native valve, the prosthetic valve including: one or more prosthetic valve leaflets, and a valve body supporting the one or more prosthetic valve leaflets, wherein at least a portion of the prosthetic valve includes a material configured to reduce tissue formation or thrombus formation along the portion of the prosthetic valve.

Example 17: The method of any example herein, in particular example 16, wherein the one or more prosthetic valve leaflets are positioned within a flow channel of the prosthetic valve, and the prosthetic valve includes a valve body having an outer surface facing outward from the flow channel and an inner surface facing towards the flow channel, and the portion of the prosthetic valve comprises the inner surface of the valve body.

Example 18: The method of any example herein, in particular example 17, wherein the outer surface includes an end portion, and the inner surface includes an end portion proximate the end portion of the outer surface, and the portion of the prosthetic valve comprises the end portion of the inner surface.

Example 19: The method of any example herein, in particular examples 16-18, wherein the portion of the prosthetic valve comprises a barrier layer adjacent to the one or more prosthetic valve leaflets.

Example 20: The method of any example herein, in particular example 19, wherein the barrier layer is configured to reduce spread of tissue formation or thrombus formation to the one or more prosthetic valve leaflets.

Example 21: A prosthetic valve configured to be deployed to a native valve, the prosthetic valve comprising: one or more prosthetic valve leaflets; and a valve body supporting the one or more prosthetic valve leaflets, wherein at least a portion of the prosthetic valve includes a frame having a rough surface.

Example 22: The prosthetic valve of any example herein, in particular example 21, wherein the rough surface comprises an unpolished surface forming a surface roughness.

Example 23: The prosthetic valve of any example herein, in particular example 22, wherein portions of the frame adjacent to the rough surface are smooth.

Example 24: The prosthetic valve of any example herein, in particular example 23, wherein the portions of the frame adjacent to the rough surface comprise polished surfaces.

Example 25: The prosthetic valve of any example herein, in particular examples 21-24, wherein the rough surface comprises a residue of a formation process of the frame.

Example 26: The prosthetic valve of any example herein, in particular examples 21-25, wherein the frame comprises a shape memory material.

Example 27: The prosthetic valve of any example herein, in particular examples 21-26, wherein the frame comprises nitinol.

Example 28: The prosthetic valve of any example herein, in particular example 27, wherein the rough surface comprises an unpolished surface roughness of the nitinol.

Example 29: The prosthetic valve of any example herein, in particular examples 21-28, wherein the rough surface comprises a microstructure.

Example 30: The prosthetic valve of any example herein, in particular examples 21-29, wherein the rough surface comprises one or more voids in a surface of the frame.

Example 31: The prosthetic valve of any example herein, in particular examples 21-30, wherein the rough surface is configured to provide friction with a portion of the native valve.

Example 32: The prosthetic valve of any example herein, in particular examples 21-31, wherein the rough surface is configured for tissue formation with the frame.

Example 33: The prosthetic valve of any example herein, in particular examples 21-32, wherein the frame comprises a portion of a sealing body for forming a seal with the native valve.

Example 34: The prosthetic valve of any example herein, in particular examples 21-33, wherein the frame comprises one or more anchors having the rough surface.

Example 35: The prosthetic valve of any example herein, in particular examples 21-34, wherein the prosthetic valve is configured to be deployed to a native mitral valve or a native tricuspid valve.

Example 36: A method comprising: deploying a prosthetic valve to a native valve, the prosthetic valve including: one or more prosthetic valve leaflets; and a valve body supporting the one or more prosthetic valve leaflets, wherein at least a portion of the prosthetic valve includes a frame having a rough surface.

Example 37: The method of any example herein, in particular example 36, wherein the rough surface comprises an unpolished surface forming a surface roughness.

Example 38: The method of any example herein, in particular example 36 or example 37, wherein the rough surface comprises a residue of a formation process of the frame.

Example 39: The method of any example herein, in particular examples 36-38, wherein the frame comprises nitinol.

Example 40: The method of any example herein, in particular example 39, wherein the rough surface comprises an unpolished surface roughness of the nitinol.

Example 41: A prosthetic valve configured to be deployed to a native valve having native valve leaflets, the prosthetic valve comprising: one or more prosthetic valve leaflets; a valve body supporting the one or more prosthetic valve leaflets, the valve body including one or more grip features configured to be positioned radially inward of one or more of the native valve leaflets; and one or more anchors configured to be positioned radially outward of the one or more of the native valve leaflets and press the one or more of the native valve leaflets towards the one or more grip features to reduce movement of the one or more of the native valve leaflets relative to the valve body.

Example 42: The prosthetic valve of any example herein, in particular example 41, wherein the one or more grip features are positioned on a sealing body for forming a seal with the native valve.

Example 43: The prosthetic valve of any example herein, in particular example 41 or example 42, wherein the one or more grip features are positioned on a frame of the valve body.

Example 44: The prosthetic valve of any example herein, in particular examples 41-43, wherein the one or more grip features are configured to reduce axial movement of the one or more of the native valve leaflets relative to the valve body.

Example 45: The prosthetic valve of any example herein, in particular examples 41-44, wherein the one or more grip features comprise a surface roughness of a frame.

Example 46: The prosthetic valve of any example herein, in particular examples 41-45 wherein the one or more grip features comprise one or more protrusions.

Example 47: The prosthetic valve of any example herein, in particular example 46, wherein the one or more protrusions extend from a frame of the valve body.

Example 48: The prosthetic valve of any example herein, in particular example 46 or example 47, wherein the one or more protrusions extend distally.

Example 49: The prosthetic valve of any example herein, in particular examples 41-48, wherein the one or more anchors are configured to press the one or more of the native valve leaflets towards the one or more grip features to deflect the one or more of the native valve leaflets between the one or more grip features and the one or more anchors.

Example 50: The prosthetic valve of any example herein, in particular examples 41-49, wherein the one or more anchors each include a tip configured to press the one or more of the native valve leaflets towards the one or more grip features.

Example 51: The prosthetic valve of any example herein, in particular example 50, wherein the tip includes a recess configured to receive the one or more grip features.

Example 52: The prosthetic valve of any example herein, in particular examples 41-51, wherein the valve body includes a frame having a plurality of struts and openings between the struts, and each of the one or more anchors are each configured to be circumferentially aligned with at least one of the openings.

Example 53: The prosthetic valve of any example herein, in particular example 52, wherein the one or more grip features comprise one or more protrusions extending from the frame towards at least one of the openings.

Example 54: The prosthetic valve of any example herein, in particular examples 41-53, wherein the valve body includes a distal end portion and a proximal end portion, and the one or more anchors extend from the distal end portion of the valve body.

Example 55: The prosthetic valve of any example herein, in particular examples 41-54, wherein the one or more anchors are configured to extend over a distal tip of one of the native valve leaflets.

Example 56: The prosthetic valve of any example herein, in particular examples 41-55, wherein the one or more anchors are configured to hook around the native valve leaflets.

Example 57: The prosthetic valve of any example herein, in particular examples 41-56, wherein the valve body includes an inner valve body and an outer valve body surrounding the inner valve body, and the one or more grip features are positioned on the outer valve body.

Example 58: The prosthetic valve of any example herein, in particular examples 41-57, wherein the one or more anchors are configured to press the one or more of the native valve leaflets towards the one or more grip features to resist a distal movement of the prosthetic valve.

Example 59: The prosthetic valve of any example herein, in particular examples 41-58, wherein the prosthetic valve comprises a prosthetic mitral valve or a prosthetic tricuspid valve, and the one or more anchors are configured to press the one or more of the native valve leaflets towards the one or more grip features to resist a ventricular movement of the prosthetic valve.

Example 60: The prosthetic valve of any example herein, in particular example 59, wherein the one or more anchors hook around the native valve leaflets to resist an atrial movement of the prosthetic valve.

Example 61: The prosthetic valve of any example herein, in particular examples 41-60, wherein the valve body includes an outer frame, and at least one of the plurality of anchors has a tip positioned distal of the outer frame.

Example 62: The prosthetic valve of any example herein, in particular example 61, wherein the tip of the at least one of the plurality of anchors protrudes to a radius that is the same or less than a radius of an outer surface of the outer frame.

Example 63: The prosthetic valve of any example herein, in particular example 61 or example 62, wherein the outer frame includes a plurality of struts forming openings between the plurality of struts, and the tip of the at least one of the plurality of anchors is positioned within one of the openings.

Example 64: The prosthetic valve of any example herein, in particular example 63, wherein the one of the openings is a distalmost opening positioned between adjacent distalmost struts of the plurality of struts.

Example 65: The prosthetic valve of any example herein, in particular examples 61-64, wherein the valve body includes a skirt and the skirt is positioned radially inward of the tip of the at least one of the plurality of anchors.

Example 66: A method comprising: deploying a prosthetic valve to a native valve, the prosthetic valve including: one or more prosthetic valve leaflets, a valve body supporting the one or more prosthetic valve leaflets, the valve body including one or more grip features configured to be positioned radially inward of one or more of the native valve leaflets, and one or more anchors configured to be positioned radially outward of the one or more of the native valve leaflets and press the one or more of the native valve leaflets towards the one or more grip features to reduce movement of the one or more of the native valve leaflets relative to the valve body.

Example 67: The method of any example herein, in particular example 66, wherein the one or more grip features are positioned on a sealing body for forming a seal with the native valve.

Example 68: The method of any example herein, in particular example 66 or example 67, wherein the one or more grip features are configured to reduce axial movement of the one or more of the native valve leaflets relative to the valve body.

Example 69: The method of any example herein, in particular examples 66-68, wherein the one or more grip features comprise a surface roughness of a frame.

Example 70: The method of any example herein, in particular examples 66-69, wherein the one or more grip features comprise one or more protrusions.

Example 71: A prosthetic valve configured to be deployed to a native valve having native valve leaflets, the prosthetic valve comprising: one or more prosthetic valve leaflets; a valve body supporting the one or more prosthetic valve leaflets; and one or more anchors extending from the valve body and configured to be positioned radially outward of one or more of the native valve leaflets to capture the one or more of the native valve leaflets, and wherein at least a portion of the prosthetic valve includes one or more grip features configured to engage a surface of the native valve upon one of the anchors failing to capture the one or more native valve leaflets.

Example 72: The prosthetic valve of any example herein, in particular example 71, wherein the one or more grip features are configured to be positioned radially inward of the one or more native valve leaflets upon one of the anchors failing to capture the one or more native valve leaflets.

Example 73: The prosthetic valve of any example herein, in particular example 71 or example 72, wherein the one or more grip features are positioned on the one or more anchors.

Example 74: The prosthetic valve of any example herein, in particular example 73, wherein the one or more grip features on one of the anchors that captures one of the native valve leaflets are configured to be positioned radially outward of the native valve leaflet that is captured.

Example 75: The prosthetic valve of any example herein, in particular example 73 or example 74, wherein the one or more grip features are positioned on a portion of the one or more anchors configured to face radially outward.

Example 76: The prosthetic valve of any example herein, in particular example 75, wherein the portion of the one or more anchors is configured to face radially outward when the one or more anchors are deployed, and is configured to face radially inward when the one or more anchors are undeployed.

Example 77: The prosthetic valve of any example herein, in particular examples 71-76, wherein the one or more grip features are positioned on the valve body.

Example 78: The prosthetic valve of any example herein, in particular example 77, wherein the one or more grip features are configured to extend radially outward from the valve body.

Example 79: The prosthetic valve of any example herein, in particular example 77 or example 78, wherein the one or more grip features are configured to protrude radially outward from the valve body to a greater radial distance than the one or more anchors.

Example 80: The prosthetic valve of any example herein, in particular examples 71-79, wherein the one or more grip features comprise one or more protrusions configured to extend distally.

Example 81: The prosthetic valve of any example herein, in particular examples 71-80, wherein the one or more grip features comprise one or more barbs.

Example 82: The prosthetic valve of any example herein, in particular examples 71-81, wherein the one or more grip features are configured to have a greater resistance to a force in a proximal direction than in a distal direction.

Example 83: The prosthetic valve of any example herein, in particular examples 71-82, wherein the anchor that fails to capture the one or more native valve leaflets is configured to be positioned radially inward of the one or more native valve leaflets.

Example 84: The prosthetic valve of any example herein, in particular examples 71-83, wherein the valve body includes a frame.

Example 85: The prosthetic valve of any example herein, in particular examples 71-84, wherein the valve body includes an inner valve body and an outer valve body.

Example 86: The prosthetic valve of any example herein, in particular example 85, wherein the outer valve body includes a frame and a skirt covering the frame.

Example 87: The prosthetic valve of any example herein, in particular examples 71-86, wherein the valve body includes a distal end portion and a proximal end portion, and the one or more anchors extend from the distal end portion of the valve body.

Example 88: The prosthetic valve of any example herein, in particular examples 71-87, wherein the one or more anchors are configured to extend over a distal tip of one of the native valve leaflets.

Example 89: The prosthetic valve of any example herein, in particular examples 71-88, wherein the one or more anchors are configured to hook around the native valve leaflets.

Example 90: The prosthetic valve of any example herein, in particular examples 71-89, wherein the prosthetic valve is configured to be deployed to a native mitral valve or a native tricuspid valve.

Example 91: A method comprising: deploying a prosthetic valve to a native valve, the prosthetic valve including: one or more prosthetic valve leaflets, a valve body supporting the one or more prosthetic valve leaflets, and one or more anchors extending from the valve body and configured to be positioned radially outward of one or more of the native valve leaflets to capture the one or more of the native valve leaflets, and wherein at least a portion of the prosthetic valve includes one or more grip features configured to engage a surface of the native valve upon one of the anchors failing to capture the one or more native valve leaflets.

Example 92: The method of any example herein, in particular example 91, wherein the one or more grip features are configured to be positioned radially inward of the one or more native valve leaflets upon one of the anchors failing to capture the one or more native valve leaflets.

Example 93: The method of any example herein, in particular example 91 or example 92, wherein the one or more grip features are positioned on the one or more anchors.

Example 94: The method of any example herein, in particular examples 91-93, wherein the one or more grip features are positioned on the valve body.

Example 95: The method of any example herein, in particular example 94, wherein the one or more grip features are configured to extend radially outward from the valve body.

Example 96: A prosthetic valve configured to be deployed to a native valve, the prosthetic valve comprising: one or more prosthetic valve leaflets; and a valve body supporting the one or more prosthetic valve leaflets and having an outer surface, wherein the outer surface of the valve body includes a channel for a pacemaker lead to be passed through.

Example 97: The prosthetic valve of any example herein, in particular example 96, wherein the outer surface of the valve body is configured to contact the native valve.

Example 98: The prosthetic valve of any example herein, in particular example 96 or example 97, wherein the outer surface of the valve body comprises a sealing surface configured to form a seal with the native valve.

Example 99: The prosthetic valve of any example herein, in particular examples 96-98, wherein the channel comprises a recess in the outer surface of the valve body.

Example 100: The prosthetic valve of any example herein, in particular examples 96-99, wherein the valve body includes a frame having a plurality of struts and openings between the struts, and the channel extends through at least one of the openings.

Example 101: The prosthetic valve of any example herein, in particular example 100, wherein the channel extends between at least two of the struts.

Example 102: The prosthetic valve of any example herein, in particular examples 96-101, wherein the valve body include a skirt, and the channel comprises a recess in the skirt.

Example 103: The prosthetic valve of any example herein, in particular examples 96-102, further comprising a plurality of anchors for anchoring the valve body to the native valve, wherein the channel is circumferentially positioned between two of the plurality of anchors.

Example 104: The prosthetic valve of any example herein, in particular example 103, wherein the channel is configured to be positioned between the outer surface of the valve body and an inner surface of the native valve.

Example 105: The prosthetic valve of any example herein, in particular examples 96-104, wherein the prosthetic valve is configured to be deployed to a native mitral valve or a native tricuspid valve.

Example 106: A method comprising: deploying a prosthetic valve to a native valve, the prosthetic valve including: one or more prosthetic valve leaflets, and a valve body supporting the one or more prosthetic valve leaflets and having an outer surface, wherein the outer surface of the valve body includes a channel for a pacemaker lead to be passed through.

Example 107: The method of any example herein, in particular example 106, wherein the outer surface of the valve body is configured to contact the native valve.

Example 108: The method of any example herein, in particular example 106 or example 107, wherein the outer surface of the valve body comprises a sealing surface configured to form a seal with the native valve.

Example 109: The method of any example herein, in particular examples 106-108, wherein the channel comprises a recess in the outer surface of the valve body.

Example 110: The method of any example herein, in particular examples 106-109, wherein the valve body includes a frame having a plurality of struts and openings between the struts, and the channel extends through at least one of the openings.

Example 111: A prosthetic valve configured to be deployed to a native valve, the prosthetic valve comprising: one or more prosthetic valve leaflets; a valve body including: an inner frame supporting the one or more prosthetic valve leaflets and having a proximal end portion and a distal end portion, and an outer frame positioned radially outward of the inner frame and having a proximal end portion and a distal end portion and an outer surface facing radially outward from the prosthetic valve, the proximal end portion of the outer frame being coupled to the proximal end portion of the inner frame, and the distal end portion of the outer frame being spaced from the inner frame with a gap; and a plurality of anchors each coupled to the inner frame and having a hook shape and extending radially outward from the inner frame, a first one of the plurality of anchors having a tip positioned radially outward of the outer frame and overlapping the outer surface, a second one of the plurality of anchors having a tip positioned distal of the outer frame and at least partially recessed radially inward of the outer surface.

Example 112: The prosthetic valve of any example herein, in particular example 111, wherein the tip of the second one of the plurality of anchors protrudes to a radius that is the same or less than a radius of the outer surface of the outer frame.

Example 113: The prosthetic valve of any example herein, in particular example 111 or example 112, wherein the outer frame includes a plurality of struts forming openings between the plurality of struts, and the tip of the second one of the plurality of anchors is positioned within one of the openings.

Example 114: The prosthetic valve of any example herein, in particular example 113, wherein the one of the openings is a distalmost opening positioned between adjacent distalmost struts of the plurality of struts.

Example 115: The prosthetic valve of any example herein, in particular examples 111-114, wherein the tip of the second one of the plurality of anchors extends to a lesser axial height in a proximal direction of the prosthetic valve than the tip of the first one of the plurality of anchors.

Example 116: The prosthetic valve of any example herein, in particular examples 111-115, wherein each of the plurality of anchors includes a drop loop, and the drop loop of the second one of the plurality of anchors protrudes axially in a distal direction of the prosthetic valve relative to a position of the drop loop of the first one of the plurality of anchors.

Example 117: The prosthetic valve of any example herein, in particular examples 111-116, wherein the valve body includes a skirt, and the skirt is positioned radially inward of the tip of the second one of the plurality of anchors.

Example 118: The prosthetic valve of any example herein, in particular examples 111-117, wherein the outer frame includes one or more grip features configured to be positioned radially inward of one or more leaflets of the native valve, and the tip of the first one of the plurality of anchors overlaps the one or more grip features to press the one or more leaflets of the native valve towards the one or more grip features to reduce movement of the one or more leaflets of the native valve relative to the valve body.

Example 119: The prosthetic valve of any example herein, in particular examples 111-118, wherein the plurality of anchors includes at least three anchors each having a tip positioned radially outward of the outer frame and overlapping the outer surface, and the plurality of anchors includes at least two anchors each having a tip positioned distal of the outer frame.

Example 120: The prosthetic valve of any example herein, in particular examples 111-119, wherein the plurality of anchors includes at least four anchors each having a tip positioned radially outward of the outer frame and overlapping the outer surface, and the plurality of anchors includes at least three anchors each having a tip positioned distal of the outer frame.

Example 121: The prosthetic valve of any example herein, in particular examples 111-120, wherein a first portion of an outer circumference of the prosthetic valve includes a plurality of anchors each having a tip positioned radially outward of the outer frame and overlapping the outer surface, and a second portion of the outer circumference includes a plurality of anchors each having a tip positioned distal of the outer frame, wherein the first portion comprises at least 180 degrees of the outer circumference.

Example 122: The prosthetic valve of any example herein, in particular example 121, wherein the first portion comprises at least 200 degrees of the outer circumference.

Example 123: The prosthetic valve of any example herein, in particular examples 111-122, wherein the outer frame has an ovalized outer profile.

Example 124: The prosthetic valve of any example herein, in particular examples 111-123, wherein each of the plurality of anchors is adapted to hook over a native valve leaflet to anchor to the native valve.

Example 125: The prosthetic valve of any example herein, in particular examples 111-124, wherein the prosthetic valve comprises a prosthetic mitral valve or a prosthetic tricuspid valve.

Example 126: A method comprising: deploying a prosthetic valve to a native valve, the prosthetic valve including: one or more prosthetic valve leaflets, a valve body including: an inner frame supporting the one or more prosthetic valve leaflets and having a proximal end portion and a distal end portion, and an outer frame positioned radially outward of the inner frame and having a proximal end portion and a distal end portion and an outer surface facing radially outward from the prosthetic valve, the proximal end portion of the outer frame being coupled to the proximal end portion of the inner frame, and the distal end portion of the outer frame being spaced from the inner frame with a gap, and a plurality of anchors each coupled to the inner frame and having a hook shape and extending radially outward from the inner frame, a first one of the plurality of anchors having a tip positioned radially outward of the outer frame and overlapping the outer surface, a second one of the plurality of anchors having a tip positioned distal of the outer frame and at least partially recessed radially inward of the outer surface.

Example 127: The method of any example herein, in particular example 126, wherein the tip of the second one of the plurality of anchors protrudes to a radius that is the same or less than a radius of the outer surface of the outer frame.

Example 128: The method of any example herein, in particular example 126 or example 127, wherein the outer frame includes a plurality of struts forming openings between the plurality of struts, and the tip of the second one of the plurality of anchors is positioned within one of the openings.

Example 129: The method of any example herein, in particular example 128, wherein the one of the openings is a distalmost opening positioned between adjacent distalmost struts of the plurality of struts.

Example 130: The method of any example herein, in particular examples 126-129, wherein the tip of the second one of the plurality of anchors extends to a lesser axial height in a proximal direction of the prosthetic valve than the tip of the first one of the plurality of anchors.

Example 131: A prosthetic valve configured to be deployed to a native valve, the prosthetic valve comprising: one or more prosthetic valve leaflets; a valve body supporting the one or more prosthetic valve leaflets; one or more hook arm anchors coupled to the valve body and each adapted to hook over a leaflet of the native valve to anchor to the native valve; and one or more clasp anchors coupled to the valve body and each adapted to clasp a portion of the native valve to anchor to the native valve.

Example 132: The prosthetic valve of any example herein, in particular example 131, wherein the valve body includes an outer surface facing radially outward, and the one or more hook arm anchors each protrude radially outward from the outer surface of the valve body.

Example 133: The prosthetic valve of any example herein, in particular example 131 or example 132, wherein the valve body includes an outer surface facing radially outward, and the one or more clasp anchors are flush with the outer surface of the valve body.

Example 134: The prosthetic valve of any example herein, in particular examples 131-133, wherein each of the one or more clasp anchors has a tip with a circumferentially planar shape.

Example 135: The prosthetic valve of any example herein, in particular examples 131-134, wherein each of the one or more clasp anchors has a drop loop and is spring biased towards the valve body.

Example 136: The prosthetic valve of any example herein, in particular examples 131-135, wherein the valve body includes an outer surface facing radially outward, and each of the one or more clasp anchors is adapted to clasp a portion of a leaflet of the native valve against the outer surface of the valve body.

Example 137: The prosthetic valve of any example herein, in particular examples 131-136, wherein the valve body includes: an inner frame supporting the one or more prosthetic valve leaflets and having a proximal end portion and a distal end portion, and an outer frame positioned radially outward of the inner frame and having a proximal end portion and a distal end portion and an outer surface facing radially outward from the prosthetic valve, the proximal end portion of the outer frame being coupled to the proximal end portion of the inner frame, and the distal end portion of the outer frame being spaced from the inner frame with a gap; and the one or more clasp anchors are coupled to the distal end portion of the inner frame.

Example 138: The prosthetic valve of any example herein, in particular examples 131-137, wherein the valve body includes: an inner frame supporting the one or more prosthetic valve leaflets and having a proximal end portion and a distal end portion, and an outer frame positioned radially outward of the inner frame and having a proximal end portion and a distal end portion and an outer surface facing radially outward from the prosthetic valve, the proximal end portion of the outer frame being coupled to the proximal end portion of the inner frame, and the distal end portion of the outer frame being spaced from the inner frame with a gap; and the one or more clasp anchors are coupled to the distal end portion of the outer frame.

Example 139: The prosthetic valve of any example herein, in particular examples 131-138, wherein the valve body includes one or more grip features configured to be positioned radially inward of one or more leaflets of the native valve.

Example 140: The prosthetic valve of any example herein, in particular example 139, wherein the one or more clasp anchors overlap the one or more grip features to press the one or more leaflets of the native valve towards the one or more grip features to reduce movement of the one or more leaflets of the native valve relative to the valve body.

Example 141: The prosthetic valve of any example herein, in particular example 139 or example 140, wherein the one or more hook arm anchors overlap the one or more grip features to press the one or more leaflets of the native valve towards the one or more grip features to reduce movement of the one or more leaflets of the native valve relative to the valve body.

Example 142: The prosthetic valve of any example herein, in particular examples 131-141, wherein the one or more clasp anchors include at least two of the clasp anchors.

Example 143: The prosthetic valve of any example herein, in particular examples 131-142, wherein a first portion of an outer circumference of the prosthetic valve includes the one or more hook arm anchors, and a second portion of the outer circumference includes the one or more clasp anchors, wherein the first portion comprises at least 180 degrees of the outer circumference.

Example 144: The prosthetic valve of any example herein, in particular example 143, wherein the first portion comprises at least 200 degrees of the outer circumference.

Example 145: The prosthetic valve of any example herein, in particular examples 131-144, wherein the prosthetic valve comprises a prosthetic mitral valve or a prosthetic tricuspid valve.

Example 146: A method comprising: deploying a prosthetic valve to a native valve, the prosthetic valve including: one or more prosthetic valve leaflets, a valve body supporting the one or more prosthetic valve leaflets, one or more hook arm anchors coupled to the valve body and each adapted to hook over a leaflet of the native valve to anchor to the native valve, and one or more clasp anchors coupled to the valve body and each adapted to clasp a portion of the native valve to anchor to the native valve.

Example 147: The method of any example herein, in particular example 146, wherein the valve body includes an outer surface facing radially outward, and the one or more hook arm anchors each protrude radially outward from the outer surface of the valve body.

Example 148: The method of any example herein, in particular example 146 or example 147, wherein the valve body includes an outer surface facing radially outward, and the one or more clasp anchors are flush with the outer surface of the valve body.

Example 149: The method of any example herein, in particular examples 146-148, wherein each of the one or more clasp anchors has a tip with a circumferentially planar shape.

Example 150: The method of any example herein, in particular examples 146-149, wherein each of the one or more clasp anchors has a drop loop and is spring biased towards the valve body.

Example 151: A prosthetic valve configured to be deployed to a native valve between an atrium and a ventricle, the prosthetic valve comprising: one or more prosthetic valve leaflets; a valve body supporting the one or more prosthetic valve leaflets and having a proximal end portion and a distal end portion; one or more hook arm anchors coupled to the valve body and each adapted to hook over a leaflet of the native valve to anchor to the native valve; and one or more support arms coupled to the valve body and each having a proximal end portion coupled to the valve body and a distal end portion protruding in a distal direction from the valve body and configured to extend into the ventricle, the one or more support arms adapted to stabilize the prosthetic valve within the native valve.

Example 152: The prosthetic valve of any example herein, in particular example 151, wherein the one or more support arms include at least two of the support arms.

Example 153: The prosthetic valve of any example herein, in particular example 151 or example 152, wherein the one or more support arms extend axially in a distal direction to a tip of the respective support arm.

Example 154: The prosthetic valve of any example herein, in particular examples 151-153, wherein the valve body includes: an inner frame supporting the one or more prosthetic valve leaflets and having a proximal end portion and a distal end portion, and an outer frame positioned radially outward of the inner frame and having a proximal end portion and a distal end portion, the proximal end portion of the outer frame being coupled to the proximal end portion of the inner frame, and the distal end portion of the outer frame being spaced from the inner frame with a gap.

Example 155: The prosthetic valve of any example herein, in particular example 154, wherein the one or more support arms are coupled to the distal end portion of the inner frame.

Example 156: The prosthetic valve of any example herein, in particular example 154 or example 155, wherein the one or more hook arm anchors are coupled to the distal end portion of the inner frame.

Example 157: The prosthetic valve of any example herein, in particular examples 151-156, wherein the one or more hook arm anchors are positioned on a first portion of the valve body extending for at least 180 degrees of an outer circumference of the prosthetic valve, and the one or more support arms are positioned on a second portion of the valve body that is opposite the first portion.

Example 158: The prosthetic valve of any example herein, in particular example 157, wherein the first portion of the valve body extends for at least 200 degrees of the outer circumference.

Example 159: The prosthetic valve of any example herein, in particular example 157 or example 158, wherein the second portion of the valve body lacks any hook arm anchors.

Example 160: The prosthetic valve of any example herein, in particular examples 157-159, wherein the prosthetic valve is a prosthetic tricuspid valve and the second portion is adapted to face a septal side of a native tricuspid valve.

Example 161: The prosthetic valve of any example herein, in particular examples 157-160, wherein the second portion of the valve body has an outer surface facing radially outward and includes a friction element for providing friction with the native valve.

Example 162: The prosthetic valve of any example herein, in particular example 161, wherein the friction element includes barbs extending parallel with a plane of the outer surface of the valve body.

Example 163: The prosthetic valve of any example herein, in particular example 161 or example 162, wherein the friction element includes a barbed sheet.

Example 164: The prosthetic valve of any example herein, in particular example 163, wherein the barbed sheet includes arms bounding one or more openings.

Example 165: The prosthetic valve of any example herein, in particular examples 151-164, wherein the one or more support arms are adapted to contact an inner heart wall within the ventricle to stabilize the prosthetic valve within the native valve, and the one or more support arms include a puncturing element for puncturing a heart wall of the ventricle to secure the one or more support arms to the heart wall.

Example 166: A method comprising: deploying a prosthetic valve to a native valve, the prosthetic valve including: one or more prosthetic valve leaflets, a valve body supporting the one or more prosthetic valve leaflets and having a proximal end portion and a distal end portion, one or more hook arm anchors coupled to the valve body and each adapted to hook over a leaflet of the native valve to anchor to the native valve, and one or more support arms coupled to the valve body and each having a proximal end portion coupled to the valve body and a distal end portion protruding in a distal direction from the valve body and configured to extend into the ventricle, the one or more support arms adapted to stabilize the prosthetic valve within the native valve.

Example 167: The method of any example herein, in particular example 166, wherein the one or more support arms include at least two of the support arms.

Example 168: The method of any example herein, in particular example 166 or example 167, wherein the one or more support arms extend axially in a distal direction to a tip of the respective support arm.

Example 169: The method of any example herein, in particular examples 166-168, wherein the valve body includes: an inner frame supporting the one or more prosthetic valve leaflets and having a proximal end portion and a distal end portion, and an outer frame positioned radially outward of the inner frame and having a proximal end portion and a distal end portion, the proximal end portion of the outer frame being coupled to the proximal end portion of the inner frame, and the distal end portion of the outer frame being spaced from the inner frame with a gap.

Example 170: The method of any example herein, in particular example 169, wherein the one or more support arms are coupled to the distal end portion of the inner frame.

Any of the features of any of the examples, including but not limited to any of the first through 170 examples referred to above, is applicable to all other aspects and examples identified herein, including but not limited to any examples of any of the first through 170 examples referred to above. Moreover, any of the features of an example of the various examples, including but not limited to any examples of any of the first through 170 examples referred to above, is independently combinable, partly or wholly with other examples described herein in any way, e.g., one, two, or three or more examples may be combinable in whole or in part. Further, any of the features of the various examples, including but not limited to any examples of any of the first through 170 examples referred to above, may be made optional to other examples. Any example of a method can be performed by a system or apparatus of another example, and any aspect or example of a system or apparatus can be configured to perform a method of another aspect or example, including but not limited to any examples of any of the first through 170 examples referred to above.

In closing, it is to be understood that although aspects of the present specification are highlighted by referring to specific examples, one skilled in the art will readily appreciate that these disclosed examples are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular examples only, and is not intended to limit the scope of systems, apparatuses, and methods as disclosed herein, which is defined solely by the claims. Accordingly, the systems, apparatuses, and methods are not limited to that precisely as shown and described.

Certain examples of systems, apparatuses, and methods are described herein, including the best mode known to the inventors for carrying out the same. Of course, variations on these described examples will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described examples in all possible variations thereof is encompassed by the systems, apparatuses, and methods unless otherwise indicated herein or otherwise clearly contradicted by context.

Groupings of alternative examples, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A prosthetic heart valve for replacing the function of a native heart valve, the prosthetic heart valve comprising:
a self-expanding frame sized for deployment within the native heart valve, the frame having an outer surface for pressing against tissue of the native heart valve;
a plurality of prosthetic valve leaflets positioned within an interior of the frame, the leaflets configured to allow flow in a first direction and to prevent flow in a second direction;
a fabric skirt covering at least a portion of the outer surface of the frame; and
a plurality of ventricular anchors extending from a downstream portion of the frame and shaped for capturing native leaflets of the native heart valve between the anchors and the outer surface of the frame;
wherein a plurality of barbs is provided along the outer surface of the frame and wherein the ventricular anchors press the native leaflets against the barbs for securing the prosthetic heart valve within the native heart valve.

2. The prosthetic heart valve of claim 1, wherein the self-expanding frame comprises an inner frame for supporting the prosthetic valve leaflets and an outer frame for sealing against tissue of the native heart valve.

3. The prosthetic heart valve of claim 2, wherein the barbs are located on the outer frame and are sized to extend through the fabric skirt for penetrating the tissue of the native heart valve.

4. The prosthetic heart valve of claim 2, wherein the inner frame is coupled to the outer frame via an intermediate component for allowing movement of the inner frame relative to the outer frame.

5. The prosthetic heart valve of claim 2, wherein the barbs are positioned only along a downstream portion of the outer frame.

6. The prosthetic heart valve of claim 2, wherein the inner frame has a substantially hourglass shape.

7. The prosthetic heart valve of claim 2, wherein the outer frame has a tapered shape such that a downstream portion of the outer frame has a smaller diameter than an intermediate portion of the outer frame.

8. The prosthetic heart valve of claim 2, wherein an outer surface of the outer frame includes a plurality of axially extending concave recesses spaced about the outer surface and for receiving a respective one of the ventricular anchors.

9. The prosthetic heart valve of claim 2, wherein the inner frame includes a first frame coupled to a second frame, the first frame including struts and surrounding a flow channel of the prosthetic heart valve, the second frame comprising struts of the plurality of ventricular anchors, a radial thickness of the struts of the plurality of ventricular anchors being less than a radial thickness of the struts of the first frame.

10. The prosthetic heart valve of claim 1, wherein the self-expanding frame comprises a plurality of struts forming expandable and collapsible cells and wherein the barbs are disposed along the struts.

11. The prosthetic heart valve of claim 1, wherein tips of the plurality of ventricular anchors are at least partially recessed radially inward of an outer surface of the prosthetic heart valve for pressing against tissue of the native heart valve.

12. The prosthetic heart valve of claim 11, wherein the frame includes a plurality of struts forming openings between the plurality of struts, and a tip of one of the plurality of anchors is positioned distal of distalmost struts of the frame and within one of the openings.

13. The prosthetic heart valve of claim 1, wherein the ventricular anchors are unequally spaced from each other.

14. The prosthetic heart valve of claim 1, wherein the plurality of ventricular anchors includes clasps that are spring biased radially inward.

15. The prosthetic heart valve of claim 14, wherein a first portion of an outer circumference of the prosthetic heart valve includes the clasps, and a second portion of the outer circumference opposite the first portion includes hook arm anchors.

16. The prosthetic heart valve of claim 1, further comprising one or more support arms coupled to the frame and each having a proximal end portion coupled to the frame and a distal end portion protruding in an axially distal direction from the frame and adapted to extend into a ventricle, the one or more support arms adapted to stabilize the prosthetic heart valve within the native heart valve.

17. The prosthetic heart valve of claim 1, wherein at least a portion of the prosthetic heart valve includes one or more grip features for engaging a surface of the native heart valve upon one of the ventricular anchors failing to capture one or more of the native leaflets.

18. The prosthetic heart valve of claim 1, wherein an outer surface of the prosthetic heart valve includes a channel for a pacemaker lead to be passed through.

19. The prosthetic heart valve of claim 1, wherein the frame has an unpolished surface forming a surface roughness for engaging the native leaflets.

20. The prosthetic heart valve of claim 1, wherein the prosthetic heart valve is sized for replacing a native tricuspid valve or mitral valve.
